(19) United States
(12) Patent Application Publication
(10) Pub. No.: US 2023/0364037 A1
(43) Pub. Date: Nov. 16, 2023

(54) CANCER THERAPEUTIC

(71) Applicant: GILLIES MCINDOE RESEARCH INSTITUTE, Wellington (NZ)

(72) Inventors: Paul Frank Davis, Wellington (NZ); Tinte Itinteang, Wellington (NZ); Reginald Walter Marsh, Tauranga (NZ); Swee Thong Tan, Wellington (NZ)

(21) Appl. No.: 18/200,251

(22) Filed: May 22, 2023

Related U.S. Application Data
(63) Continuation of application No. 16/482,056, filed on Jul. 30, 2019, now abandoned.
(60) Provisional application No. 62/452,479, filed on Jan. 31, 2017.

(51) Int. Cl. A61K 31/138; A61P 35/00; A61K 9/00; A61K 31/12; A61K 31/155; A61K 31/165; A61K 31/415; A61K 31/4178; A61K 31/551; A61K 31/616; A61K 31/65

(52) U.S. Cl. CPC A61K 31/138; A61P 35/00; A61K 9/0053; A61K 31/12; A61K 31/155; A61K 31/165; A61K 31/415; A61K 31/4178; A61K 31/551; A61K 31/616; A61K 31/65

(57) ABSTRACT

The present invention provides novel drug combinations, pharmaceutical compositions, as well as methods involving novel therapeutic regimes for targeting cancer stem cells, as well as methods of use thereof, for the treatment and management of cancerous and non-cancerous tumours in a patient. In particular, the present invention provides novel drug combinations and pharmaceutical compositions that target components of the Renin Angiotensin System shown to expressed by cancer stem cells.

### TECHNICAL FIELD

The present invention relates to novel therapeutic regimes including, for example, drug combinations, pharmaceutical compositions and methods useful for preventing, treating, and/or managing cancer, as well as articles of manufacture and kits comprising therapeutic regimes, all of which are useful in targeting cancer stem cells present in cancerous and non-cancerous tumours.

### BACKGROUND OF THE INVENTION

The following includes information that may be useful in understanding the present invention. It is not an admission that any of the information, publications or documents specifically or implicitly referenced herein is prior art, or essential, to the presently described or claimed inventions. All publications and patents mentioned herein are hereby incorporated herein by reference in their entirety.

Cancer stem cells (CSCs), the proposed origin of cancer, have been identified in many types of cancer including oral cavity squamous cell carcinoma (OCSCC)¹, malignant melanoma (MM)² and glioblastoma multiforme (GBM)³. These CSCs resist radiotherapy and chemotherapy and they go into a slow cycle state during these treatments⁴. This could explain the observation that cancers that have gone into remission following such treatments return many years later.

Applicants' research has identified CSCs in 12 different types of cancer⁵ including tongue SCC⁶, buccal mucosal SCC⁷, malignant melanoma and GBM⁸.

The Renin Angiotensin System (RAS) is classically associated with blood pressure regulation. Physiologically, the RAS consists of Angiotensinogen which is converted to Angiotensin I (ATI), by renin. ATI is then converted to angiotensin II (ATII), by Angiotensin Converting Enzyme (ACE). ATII, the active peptide, acts on its receptors, Angiotensin II Receptors 1 (ATIIR1) and Angiotensin II Receptors 2 (ATIIR2). Renin is formed by the cleavage of its inactive precursor, pro-renin, by a number of enzymes including Cathepsin⁹, to active renin, as well as by binding to the Pro-Renin Receptor (PRR)¹⁰. Cyclo-oxygenase-2 (COX2) causes the upregulation of PRR¹¹. β-blockers reduce the production of Pro-Renin¹². Furthermore, Insulin Growth Factor (IGF) which acts on Insulin Growth Factor Receptor-1 (IGFR-1) promotes the conversion of Pro-Renin to active Renin¹³, as well as being implicated in cancer metastasis¹⁴. Metformin is a known inhibitor of the IGFR-1 pathway¹⁵. The action of ATII on ATIIR1 and ATIIR2 can be blocked by Angiotensin Receptor Blockers (ATRBs) (FIG. 1).

The peptides derived from the RAS have been implicated in tumour progression¹⁶ and the expression of PRR has been associated with a poorer prognosis in cancer patients¹⁷.

Applicants have demonstrated the expression of components of the RAS, namely the PRR, ACE, ATIIR1 and ATIIR2 in the CSC population in 12 types of cancer⁵ including tongue SCC¹⁸, buccal mucosal SCC¹⁹, skin SCC, MM and GBM²⁰. This coupled with the understanding of the regulation of the RAS including the expression and function of cathepsin²¹ and IGFR-1 pathway¹⁴ led Applicants to propose CSCs as a novel therapeutic target for cancer by modulation of the RAS using various cocktails of existing drugs that are commonly used for other medical conditions¹⁸⁻²⁰.

The present invention is directed to novel therapeutic regimes for the prevention, treatment and/or management of cancer and benign tumours, including drug combinations, pharmaceutical compositions as well as kits and articles of manufacture comprising the same.

### SUMMARY OF THE INVENTION

The inventions described and claimed herein have many attributes and embodiments including, but not limited to, those set forth or described or referenced in this Summary of the Invention. It is not intended to be all-inclusive and the inventions described and claimed herein are not limited to or by the features of or embodiments identified in this Summary of the Invention, which is included for purposes of illustration only and not restriction.

Applicants have surprisingly identified that certain drug combinations and pharmaceutical compositions are particularly useful in treating or managing cancer and non-cancerous tumours in a patient.

In an aspect of the present invention there is provided a drug combination comprising a therapeutically effective amount of two or more compounds selected from a COX-2 inhibitor, a beta-blocker, a cathepsin inhibitor, an IGFR-1 pathway inhibitor, an angiotensin converting enzyme inhibitor or an angiotensin receptor blocker, a renin inhibitor, as well as combinations thereof.

In another aspect of the present invention there is provided a pharmaceutical composition comprising a therapeutically effective amount of two or more compounds selected from a COX-2 inhibitor, a beta-blocker, a cathepsin inhibitor, an IGFR-1 pathway inhibitor, an angiotensin converting enzyme inhibitor or an angiotensin receptor blocker, a renin inhibitor, as well as combinations thereof, together with a pharmaceutically effective carrier.

In another aspect of the present invention there is provided a drug combination or a pharmaceutical composition comprising Aspirin, Propanolol and Curcumin. In a related aspect, the drug combination or a pharmaceutical composition comprises acetylsalicylic acid, (RS)-1-(1-methylethylamino)-3-(1-naphthyloxy)propan-2-ol and (1E,6E)-1,7-Bis(4-hydroxy-3-methoxyphenyl)hepta-1,6-diene-3,5-dione.

In a further aspect of the present invention there is provided a drug combination or pharmaceutical composition comprising Aspirin, Curcumin and Aliskiren. In a related aspect, the drug combination or a pharmaceutical composition comprises acetylsalicylic acid, (1E,6E)-1,7-Bis(4-hydroxy-3-methoxyphenyl)hepta-1,6-diene-3,5-dione and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide.

In yet a further aspect of the present invention there is provided a drug combination or a pharmaceutical composition comprising Celecoxib, Propanalol and Curcumin. In a related aspect, the drug combination or pharmaceutical composition comprises 4-[5-(4-methylphenyl)-3-(trifluoromethyl)pyrazol-1-yl]benzenesulfonamide, (RS)-1-(1-methylethylamino)-3-(1-naphthyloxy)propan-2-ol and (1E,6E)-1,7-Bis(4-hydroxy-3-methoxyphenyl)hepta-1,6-diene-3,5-dione.

In yet a further aspect of the present invention there is provided a drug combination or a pharmaceutical composition comprising Celecoxib, Curcumin and Aliskiren. In a related aspect, the drug combination or pharmaceutical composition comprises 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide.

In yet another aspect of the present invention there is provided a drug combination or a pharmaceutical composition comprising Curcumin, Propanolol, Aspirin and Quinapril. In a related aspect, the drug combination or pharmaceutical composition comprises (1E,6E)-1,7-Bis(4-hydroxy-3-methoxyphenyl)hepta-1,6-diene-3,5-dione, (RS)-1-(1-methylethylamino)-3-(1-naphthyloxy)propan-2-ol, acetylsalicylic acid and [3S-[2[R*(R)],3R*]]-2-[2-[[1-Ethoxycarbonyl)-3-phenylpropyl]amino]-1-oxopropyl]-1,2,3,4-tetrahydro-3-isoquinolinecarboxylic acid monohydrochloride.

In a further aspect of the present invention there is provided a drug combination or pharmaceutical composition comprising Aliskiren, Celecoxib and Curcumin. In a related aspect the drug combination or pharmaceutical composition comprises (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide and (1E,6E)-1,7-Bis(4-hydroxy-3-methoxyphenyl)hepta-1,6-diene-3,5-dione.

In yet a further aspect of the present invention there is provided a drug combination or pharmaceutical composition comprising Aliskiren, Celecoxib, Curcumin and Metformin. In a related aspect the drug combination or pharmaceutical composition comprises (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione and N,N-dimethylimidodicarbonimidic diamide.

In yet a further aspect of the present invention there is provided a drug combination or pharmaceutical composition comprising Aliskiren, Celecoxib, Curcumin, Metformin and Propanolol. In a related aspect the drug combination or pharmaceutical composition comprises (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide and (RS)-1-(1-methylethylamino)-3-(1-naphthyloxy)propan-2-ol.

In yet a further aspect of the present invention there is provided a drug combination or pharmaceutical composition comprising Aliskiren, Celecoxib, Curcumin, Metformin, Propanolol and Cilazapril. In a related aspect the drug combination or pharmaceutical composition comprises (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide, (RS)-1-(1-methylethylamino)-3-(1-naphthyloxy)propan-2-ol and (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid.

In yet a further aspect of the present invention there is provided a method for preventing, treating and/or managing cancer or a non-cancerous tumour in a patient in need thereof, the method comprising administering to the patient a therapeutically effective amount of one or more drug combinations or a pharmaceutical compositions as described herein.

In another aspect of the present invention, there is provided a method for preventing, treating and/or managing cancer or a non-cancerous tumour in a patient in need thereof, the method comprising the steps of administering to the patient:
(i) a drug combination or a pharmaceutical composition comprising (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide and (1E,6E)-1,7-Bis(4-hydroxy-3-methoxyphenyl)hepta-1,6-diene-3,5-dione via an oral route of administration for a period of about two weeks; and
(ii) a drug combination or a pharmaceutical composition comprising (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione and N,N-dimethylimidodicarbonimidic diamide via an oral route of administration for a period of about another two weeks to about another four weeks;
(iii) a drug combination or a pharmaceutical composition comprising (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide and (RS)-1-(1-methylethylamino)-3-(1-naphthyloxy)propan-2-ol via an oral route of administration for a period of about a further two weeks to about a further four weeks; and
(iv) a drug combination or a pharmaceutical composition comprising (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide, (RS)-1-(1-methylethylamino)-3-(1-naphthyloxy)propan-2-ol and (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid via an oral route of administration for a period of about another two weeks to about another six weeks or longer, as required.

In yet another aspect of the present invention there is provided a drug combination or a pharmaceutical composition as described herein, for use in preventing, treating and/or managing cancer or a non-cancerous tumour in a patient in need thereof.

In yet another aspect of the present invention there is provided use of a drug combination or a pharmaceutical composition as descibed herein, in the manufacture of a medicament for preventing, treating and/or managing cancer or a non-cancerous tumour in a patient in need thereof.

In yet another aspect of the present invention there is provided an article of manufacture comprising one or more of the drug combinations or pharmaceutical compositions as described herein, and optionally instructions for how to prevent, treat and/or manage cancer or a non-cancerous tumour in a patient in need thereof.

In yet another aspect of the present invention there is provided a kit comprising one or more of the drug combinations or pharmaceutical compositions as described herein, and optionally instructions for how to prevent, treat and/or manage cancer or a non-cancerous tumour in a patient in need thereof.

### BRIEF DESCRIPTION OF THE FIGURES

FIG. 1 shows the main pathways associated with the Renin-Angiotensin System. ACE: Angiotensin Converting Enzyme; ACEi: Angiotensin Converting Enzyme inhibitors; Cox2i: COX-2 inhibitors; β-blockers: beta-Blockers; ATIIR2: Angiotensin II Receptor 2; ATIIR1: Angiotensin II Receptor 1; PRR: Pro-Renin Receptor [also referred to herein as Renin Receptor (RR)]; ATRB: angiotensin receptor blocker; IGF/IGFR-1: Insulin Growth Factor Receptor-1 Pathway; X: major blockades; +: major promoting steps.

### SELECTED DEFINITIONS

Unless defined otherwise, all technical and scientific terms used herein have the same meaning as commonly understood to one of ordinary skill in the art to which the inventions belong. Although any assays, methods, devices and materials similar or equivalent to those described herein can be used in the practice or testing of the invention, various assays, methods, devices and materials are now described.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

As used in this specification, the terms “comprises”, “comprising”, and similar words, are not to be interpreted in an exclusive or exhaustive sense. In other words, they are intended to mean “including, but not limited to”.

As used in this specification, the term “Aspirin” includes acetylsalicylic acid, a known analgesic used to treat pain, inflammation and fever.

As used in this specification, the term “Celecoxib” includes 4-[5-(4-methylphenyl)-3-(trifluoromethyl)pyrazol-1-yl]benzenesulfonamide, a known COX-2 inhibitor.

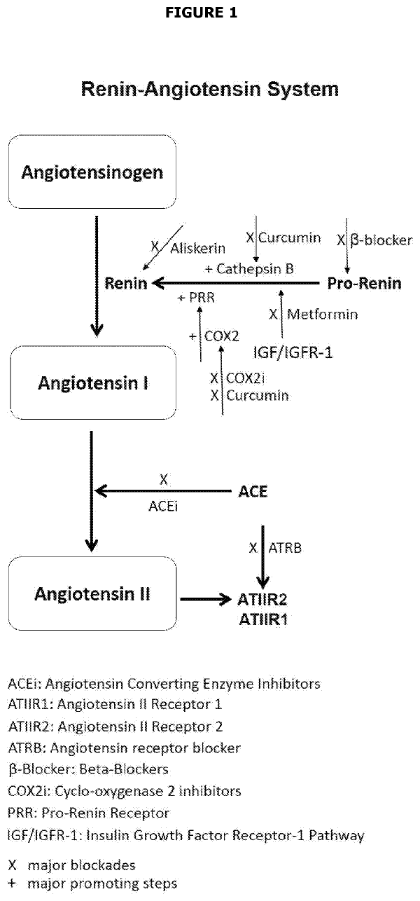

As used in this specification, the term “Propranolol” includes (RS)-1-(1-methylethylamino)-3-(1-naphthyloxy)propan-2-ol, a type of beta-blocker known to reduce the production of pro-renin (refer to FIG. 1).

As used in this specification, the term “Metformin” includes N,N-dimethylimidodicarbonimidic diamide, a known inhibitor of the IGFR-1 pathway implicated in the conversion of pro-renin to renin (refer to FIG. 1).

As used in this specification, the term “Curcumin” includes (1E,6E)-1,7-Bis(4-hydroxy-3-methoxyphenyl)hepta-1,6-diene-3,5-dione, a natural phenol and known inhibitor of cathepsin (refer to FIG. 1).

As used in this specification, the term “Cilazapril” includes (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid, a known angiotensin converting enzyme inhibitor (refer to FIG. 1).

As used in this specification, the term “Aliskiren” includes (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, a known renin inhibitor (refer to FIG. 1).

As used in this specification, the term “Piperine” includes 1-[5-(1,3-Benzodioxol-5-yl)-1-oxo-2,4-pentadienyl]piperidine, which increases the bioavailability of Curcumin.

As used in this specification, the term “Omeprazole” includes 5-methoxy-2-[(4-methoxy-3,5-dimethylpyridin-2-yl)methylsulfinyl]-1H-benzimidazole, which decreases the likelihood of peptide ulcers caused by non-steroidal anti-inflammatory drugs (NSAIDs) including (e.g.,) Aspirin.

As used in this specification, the term “Losartan” includes 2-butyl-4-chloro-1-{[2′-(1H-tetrazol-5-yl)biphenyl-4-yl]methyl}-1H-imidazol-5-yl)methanol.

As used in this specificatoin, the terms “Quinapril” and “Accupril” may be used interchangeably and includes [3S-[2[R*(R)],3R*]]-2-[2-[[1-Ethoxycarbonyl)-3-phenylpropyl]amino]-1-oxopropyl]-1,2,3,4-tetrahydro-3-isoquinolinecarboxylic acid monohydrochloride.

As used herein, the term “effective amount”, “prophylactically effective amount” and “therapeutically effective amount” refers to the amount of a therapy that is sufficient to result in the prevention of the development, recurrence, or onset of a disease or condition and one or more symptoms thereof, to enhance or improve the prophylactic effect(s) of another therapy, reduce the severity, the duration of disease, ameliorate one or more symptoms of the disease or condition, prevent the advancement of the disease or condition, cause regression of the disease or condition, and/or enhance or improve the therapeutic effect(s) of another therapy.

As used herein, the terms “manage”, “managing”, and “management” in the context of the administration of a therapy to a subject refer to the beneficial effects that a subject derives from a therapy (e.g., a prophylactic or therapeutic agent) or a combination of therapies, while not resulting in a cure of the disease or condition. In certain examples, a subject is administered one or more therapies (e.g., one or more prophylactic or therapeutic agents) to “manage” the disease or condition so as to prevent the progression or worsening of the disease or condition.

As used herein, the terms “prevent”, “preventing” and “prevention” in the context of the administration of a therapy to a subject refers to the prevention or inhibition of the recurrence, onset, and/or development of a disease or condition or a symptom thereof in a subject resulting from the administration of a therapy (e.g., a prophylactic or therapeutic agent), or a combination of therapies (e.g., a combination of prophylactic or therapeutic agents).

As used herein, the term “marker” or “biomarker” in the context of a tissue means any antigen, molecule or other chemical or biological entity that is specifically found in or on a tissue that it is desired to be identified or identified in or on a particular tissue affected by a disease or disorder, for example cancer. In specific examples, the marker is a cell surface antigen that is differentially or preferentially expressed by specific cell types. In specific examples, the marker is a nuclear antigen that is differentially or preferentially expressed by specific cell types. In specific examples the marker is an intracellular antigen that is differentially or preferentially expressed by specific cell types.

As used herein, the term “prophylactic agent” refers to any molecule, compound, and/or substance that is used for the purpose of preventing fibrosis. Examples of prophylactic agents include, but are not limited to, proteins, immunoglobulins (e.g., multi-specific Igs, single chain Igs, Ig fragments, polyclonal antibodies and their fragments, monoclonal antibodies and their fragments), antibody conjugates or antibody fragment conjugates, peptides (e.g., peptide receptors, selectins), binding proteins, proliferation based therapy, and small molecule drugs.

As used herein, the term “therapeutic agent” refers to any molecule, compound, and/or substance that is used for the purpose of treating and/or managing a disease or disorder. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), peptides (e.g., peptide receptors, selectins), binding proteins, biologics, proliferation-based therapy agents, hormonal agents, radioimmunotherapies, targeted agents, epigenetic therapies, differentiation therapies, biological agents, and small molecule drugs.

As used herein, the terms “therapies” and “therapy” can refer to any method(s), composition(s), and/or agent(s) that can be used in the prevention, treatment and/or management of cancer or one or more symptoms thereof.

As used herein, the terms “treat”, “treatment” and “treating” in the context of the administration of a therapy to a subject refers to the reduction or inhibition of the progression and/or duration of cancer, the reduction or amelioration of the severity of cancer, and/or the amelioration of one or more symptoms thereof resulting from the administration of one or more therapies.

The term “sample” or “biological sample” as used herein means any sample taken or derived from a subject. Such a sample may be obtained from a subject, or may be obtained from biological materials intended to be provided to the subject. For example, a sample may be obtained from blood being assessed, for example, to investigate cancer in a subject. Included are samples taken or derived from any subjects such as from normal healthy subjects and/or healthy subjects for whom it is useful to understand their cancer status. Preferred samples are biological fluid samples. The term “biological fluid sample” as used herein refers to a sample of bodily fluid obtained for the purpose of, for example, diagnosis, prognosis, classification or evaluation of a subject of interest, such as a patient.

The sample may be any sample known in the art in which embryonic stem cells may be detected. Included are any body fluids such as a whole blood sample, plasma, serum, ovarian follicular fluid sample, seminal fluid sample, cerebrospinal fluid, saliva, sputum, urine, pleural effusions, interstitial fluid, synovial fluid, lymph, tears, for example, although whole blood sample, plasma and serum are particularly suited for use in this invention. In addition, one of skill in the art would realise that certain body fluid samples would be more readily analysed following a fractionation or purification procedure, for example, separation of whole blood into serum or plasma components.

The term “patient” and “subject” as used herein is preferably a mammal and includes human, and non-human mammals such as cats, dogs, horses, cows, sheep, deer, mice, rats, primates (including gorillas, rhesus monkeys and chimpanzees), possums and other domestic farm or zoo animals. Thus, the assays, methods and kits described herein have application to both human and non-human animals, in particular, and without limitation, humans, primates, farm animals including cattle, sheep, goats, pigs, deer, alpacas, llamas, buffalo, companion and/or pure bred animals including cats, dogs and horses. Preferred subjects are humans, and most preferably “patients” who as used herein refer to living humans who may receive or are receiving medical care or assessment for a disease or condition. Further, while a subject is preferably a living organism, the invention described herein may be used in post-mortem analyses as well.

A level “higher” or “lower” than a control, or a “change” or “deviation” from a control (level) in one embodiment is statistically significant. A higher level, lower level, deviation from, or change from a control level or mean or historical control level can be considered to exist if the level differs from the control level by about 5% or more, by about 10% or more, by about 20% or more, or by about 50% or more compared to the control level. Statistically significant may alternatively be calculated as P≤0.05. Higher levels, lower levels, deviation, and changes can also be determined by recourse to assay reference limits or reference intervals. These can be calculated from intuitive assessment or non-parametric methods. Overall, these methods may calculate the 0.025, and 0.975 fractiles as 0.025* (n+1) and 0.975 (n+1). Such methods are well known in the art. Presence of a marker absent in a control may be seen as a higher level, deviation or change. Absence of a marker present in a control may be seen as a lower level, deviation or change.

As used herein, the term “Renin-Angiotensin System (RAS)” or “Renin-Angiotensin-Aldosterone System (RAAS)” is a hormone system that regulates blood pressure and fluid balance. The wider pathway associated with RAS also includes the Pro/Renin Receptor System (PRRS) and the associated bypass pathways. By way of example, refer to FIG. 1. There are a number of known drugs which target the RAS including PRRS, as described in more detail below.

### DETAILED DESCRIPTION

The present invention is based on the discovery that non-obvious drug combinations are surprisingly useful for treating and/or preventing cancer including the recurrence of cancer. The drug combinations including pharmaceutical compositions and formulations according to the present invention target components of the renin-angiotensin system (RAS) for which the Applicants have previously demonstrated is expressed by cancer stem cell populations associated with diverse tumour types. These cancer stem cells therefore represent a novel therapeutic target (refer to WO2016024870, which is incorporated herein by reference) for which the combinations, compositions and formulations described herein are useful.

Accordingly, in one aspect of the present invention there is provided a drug combination comprising a therapeutically effective amount of two or more compounds selected from a COX-2 inhibitor, a beta-blocker, a cathepsin inhibitor, an IGFR-1 pathway inhibitor, an angiotensin converting enzyme inhibitor, a renin inhibitor, as well as combinations thereof.

In an example according to this aspect of the present invention, the drug combination comprises a COX-2 inhibitor, a beta-blocker, a cathepsin inhibitor, an IGFR-1 pathway inhibitor, an angiotensin converting enzyme inhibitor and a renin inhibitor.

In another example according to this aspect of the present invention, the drug combination comprises a COX-2 inhibitor, a beta-blocker, a cathepsin inhibitor, an angiotensin converting enzyme inhibitor and a renin inhibitor.

In another example according to this aspect of the present invention, the drug combination comprises a COX-2 inhibitor, a beta-blocker, a cathepsin inhibitor, an IGFR-1 pathway inhibitor and a renin inhibitor.

In another example according to this aspect of the present invention, the drug combination comprises a COX-2 inhibitor, a beta-blocker, an IGFR-1 pathway inhibitor, an angiotensin converting enzyme inhibitor and a renin inhibitor.

In another example according to this aspect of the present invention, the drug combination comprises a COX-2 inhibitor, a cathepsin inhibitor, an IGFR-1 pathway inhibitor, an angiotensin converting enzyme inhibitor and a renin inhibitor.

In another example according to this aspect of the present invention, the drug combination comprises a beta-blocker, a cathepsin inhibitor, an IGFR-1 pathway inhibitor, an angiotensin converting enzyme inhibitor and a renin inhibitor.

In yet another example according to this aspect of the present invention, the drug combination comprises a COX-2 inhibitor, a beta-blocker, a cathepsin inhibitor and a renin inhibitor.

In yet another example according to this aspect of the present invention, the drug combination comprises a COX-2 inhibitor, a beta-blocker, a cathepsin inhibitor and an angiotensin converting enzyme inhibitor.

In yet another example according to this aspect of the present invention, the drug combination comprises a COX-2 inhibitor, a beta-blocker, a cathepsin inhibitor and an IGFR-1 pathway inhibitor.

In yet another example according to this aspect of the present invention, the drug combination comprises a COX-2 inhibitor, a beta-blocker, an angiotensin converting enzyme inhibitor and a renin inhibitor.

In yet another example according to this aspect of the present invention, the drug combination comprises a COX-2 inhibitor, a beta-blocker, an IGFR-1 pathway inhibitor and a renin inhibitor.

In yet another example according to this aspect of the present invention, the drug combination comprises a COX-2 inhibitor, a beta-blocker, an angiotensin converting enzyme inhibitor and an IGFR-1 pathway inhibitor.

In yet another example according to this aspect of the present invention, the drug combination comprises a COX-2 inhibitor, a cathepsin inhibitor, an angiotensin converting enzyme inhibitor and a renin inhibitor.

In yet another example according to this aspect of the present invention, the drug combination comprises a COX-2 inhibitor, a cathepsin inhibitor, an IGFR-1 pathway inhibitor and a renin inhibitor.

In yet another example according to this aspect of the present invention, the drug combination comprises a COX-2 inhibitor, a cathepsin inhibitor, an angiotensin converting enzyme inhibitor and an IGFR-1 pathway inhibitor.

In yet another example according to this aspect of the present invention, the drug combination comprises a COX-2 inhibitor, an angiotensin converting enzyme inhibitor, an IGFR-1 pathway inhibitor and a renin inhibitor.

In yet another example according to this aspect of the present invention, the drug combination comprises a beta-blocker, a cathepsin inhibitor, an angiotensin converting enzyme inhibitor and a renin inhibitor.

In yet another example according to this aspect of the present invention, the drug combination comprises a beta-blocker, a cathepsin inhibitor, an IFGR-1 pathway inhibitor and a renin inhibitor.

In yet another example according to this aspect of the present invention, the drug combination comprises a beta-blocker, a cathepsin inhibitor, an angiotensin converting enzyme inhibitor and an IGFR-1 pathway inhibitor.

In yet another example according to this aspect of the present invention, the drug combination comprises a beta-blocker, an angiotensin converting enzyme inhibitor, an IGFR-1 pathway inhibitor and a renin inhibitor.

In yet another example according to this aspect of the present invention, the drug combination comprises a cathepsin inhibitor, an angiotensin converting enzyme inhibitor, an IGFR-1 pathway inhibitor and a renin inhibitor.

In a further example according to this aspect of the present invention, the drug combination comprises a COX-2 inhibitor, a beta-blocker and a cathepsin inhibitor.

In a further example according to this aspect of the present invention, the drug combination comprises a COX-2 inhibitor, a beta-blocker and a renin inhibitor.

In a further example according to this aspect of the present invention, the drug combination comprises a COX-2 inhibitor, a beta-blocker and an angiotensin converting enzyme inhibitor.

In a further example according to this aspect of the present invention, the drug combination comprises a COX-2 inhibitor, a beta-blocker, and an IGFR-1 pathway inhibitor.

In a further example according to this aspect of the present invention, the drug combination comprises a COX-2 inhibitor, a cathepsin inhibitor and a renin inhibitor.

In a further example according to this aspect of the present invention, the drug combination comprises a COX-2 inhibitor, a cathepsin inhibitor and an angiotensin converting enzyme inhibitor.

In a further example according to this aspect of the present invention, the drug combination comprises a COX-2 inhibitor, a cathepsin inhibitor and an IGFR-1 pathway inhibitor.

In a further example according to this aspect of the present invention, the drug combination comprises a COX-2 inhibitor, an angiotensin converting enzyme inhibitor and a renin inhibitor.

In a further example according to this aspect of the present invention, the drug combination comprises a COX-2 inhibitor, an IGFR-1 pathway inhibitor and a renin inhibitor.

In a further example according to this aspect of the present invention, the drug combination comprises a COX-2 inhibitor, an IGFR-1 pathway inhibitor, and an angiotensin converting enzyme inhibitor.

In a further example according to this aspect of the present invention, the drug combination comprises a beta-blocker, a cathepsin inhibitor and a renin inhibitor.

In a further example according to this aspect of the present invention, the drug combination comprises a beta-blocker, a cathepsin inhibitor and an angiotensin converting enzyme inhibitor.

In a further example according to this aspect of the present invention, the drug combination comprises a beta-blocker, a cathepsin inhibitor and an IGFR-1 pathway inhibitor.

In a further example according to this aspect of the present invention, the drug combination comprises a beta-blocker, an angiotensin converting enzyme inhibitor and a renin inhibitor.

In a further example according to this aspect of the present invention, the drug combination comprises a beta-blocker, an IGFR-1 pathway inhibitor and a renin inhibitor.

In a further example according to this aspect of the present invention, the drug combination comprises a beta-blocker, an IGFR-1 pathway inhibitor and an angiotensin converting enzyme inhibitor.

In a further example according to this aspect of the present invention, the drug combination comprises a cathepsin inhibitor, an angiotensin converting enzyme inhibitor and a renin inhibitor.

In a further example according to this aspect of the present invention, the drug combination comprises a cathepsin inhibitor, an IGFR-1 pathway inhibitor and a renin inhibitor.

In a further example according to this aspect of the present invention, the drug combination comprises a cathepsin inhibitor, an IGFR-1 pathway inhibitor and an angiotensin converting enzyme inhibitor.

In a further example according to this aspect of the present invention, the drug combination comprises an IGFR-1 pathway inhibitor, an angiotensin converting enzyme inhibitor and a renin inhibitor.

In yet a further example according to this aspect of the present invention, the drug combination comprises a COX-2 inhibitor and a beta-blocker.

In yet a further example according to this aspect of the present invention, the drug combination comprises a COX-2 inhibitor and a cathepsin inhibitor.

In yet a further example according to this aspect of the present invention, the drug combination comprises a COX-2 inhibitor and a renin inhibitor.

In yet a further example according to this aspect of the present invention, the drug combination comprises a COX-2 inhibitor and an angiotensin converting enzyme inhibitor.

In yet a further example according to this aspect of the present invention, the drug combination comprises a COX-2 inhibitor and an IGFR-1 pathway inhibitor.

In yet a further example according to this aspect of the present invention, the drug combination comprises a beta-blocker and a cathepsin inhibitor.

In yet a further example according to this aspect of the present invention, the drug combination comprises a beta-blocker and a renin inhibitor.

In yet a further example according to this aspect of the present invention, the drug combination comprises a beta-blocker and an angiotensin converting enzyme inhibitor.

In yet a further example according to this aspect of the present invention, the drug combination comprises a beta-blocker and an IGFR-1 pathway inhibitor.

In yet a further example according to this aspect of the present invention, the drug combination comprises a cathepsin inhibitor and a renin inhibitor.

In yet a further example according to this aspect of the present invention, the drug combination comprises a cathepsin inhibitor and angiotensin converting enzyme inhibitor.

In yet a further example according to this aspect of the present invention, the drug combination comprises a cathepsin inhibitor and an IGFR-1 pathway inhibitor.

In yet a further example according to this aspect of the present invention, the drug combination comprises an angiotensin converting enzyme inhibitor and a renin inhibitor.

In yet a further example according to this aspect of the present invention, the drug combination comprises an IGFR-1 pathway inhibitor and a renin inhibitor.

In yet a further example according to this aspect of the present invention, the drug combination comprises an IGFR-1 pathway inhibitor and an angiotensin converting enzyme inhibitor.

The drug combinations according to the present invention may be formulated as one or more pharmaceutical compositions for simultaneous, separate and/or sequential administration to a patient in need thereof. By way of illustration only, where the drug combination comprises (e.g.) a COX-2 inhibitor and a beta-blocker, the COX-2 inhibitor and beta-blocker may be formulated as discrete pharmaceutical compositions for separate and/or sequential administration to a patient in need thereof, or in the same pharmaceutical composition for simultaneous administration to a patient in need thereof. However, formulation of the COX-2 inhibitor and a beta-blocker in separate pharmaceutical compositions does not preclude simulataneous administration to a patient.

Accordingly, in another aspect of the present invention there is provided a pharmaceutical composition comprising a therapeutically effective amount of two or more compounds selected from a COX-2 inhibitor, a beta-blocker, a cathepsin inhibitor, an IGFR-1 pathway inhibitor, an angiotensin converting enzyme inhibitor, a renin inhibitor, as well as combinations thereof, together with a pharmaceutically effective carrier.

In an example according to this aspect of the present invention, the pharmaceutical composition comprises a COX-2 inhibitor, a beta-blocker, a cathepsin inhibitor, an IGFR-1 pathway inhibitor, an angiotensin converting enzyme inhibitor and a renin inhibitor.

In another example according to this aspect of the present invention, the pharmaceutical composition comprises a COX-2 inhibitor, a beta-blocker, a cathepsin inhibitor, an angiotensin converting enzyme inhibitor and a renin inhibitor.

In another example according to this aspect of the present invention, the pharmaceutical composition comprises a COX-2 inhibitor, a beta-blocker, a cathepsin inhibitor, an IGFR-1 pathway inhibitor and a renin inhibitor.

In another example according to this aspect of the present invention, the pharmaceutical composition comprises a COX-2 inhibitor, a beta-blocker, an IGFR-1 pathway inhibitor, an angiotensin converting enzyme inhibitor and a renin inhibitor.

In another example according to this aspect of the present invention, the pharmaceutical composition comprises a COX-2 inhibitor, a cathepsin inhibitor, an IGFR-1 pathway inhibitor, an angiotensin converting enzyme inhibitor and a renin inhibitor.

In another example according to this aspect of the present invention, the pharmaceutical composition comprises a beta-blocker, a cathepsin inhibitor, an IGFR-1 pathway inhibitor, an angiotensin converting enzyme inhibitor and a renin inhibitor.

In yet another example according to this aspect of the present invention, the pharmaceutical composition comprises a COX-2 inhibitor, a beta-blocker, a cathepsin inhibitor and a renin inhibitor.

In yet another example according to this aspect of the present invention, the pharmaceutical composition comprises a COX-2 inhibitor, a beta-blocker, a cathepsin inhibitor and an angiotensin converting enzyme inhibitor.

In yet another example according to this aspect of the present invention, the pharmaceutical composition comprises a COX-2 inhibitor, a beta-blocker, a cathepsin inhibitor and an IGFR-1 pathway inhibitor.

In yet another example according to this aspect of the present invention, the pharmaceutical composition comprises a COX-2 inhibitor, a beta-blocker, an angiotensin converting enzyme inhibitor and a renin inhibitor.

In yet another example according to this aspect of the present invention, the pharmaceutical composition comprises a COX-2 inhibitor, a beta-blocker, an IGFR-1 pathway inhibitor and a renin inhibitor.

In yet another example according to this aspect of the present invention, the pharmaceutical composition comprises a COX-2 inhibitor, a beta-blocker, an angiotensin converting enzyme inhibitor and an IGFR-1 pathway inhibitor.

In yet another example according to this aspect of the present invention, the pharmaceutical composition comprises a COX-2 inhibitor, a cathepsin inhibitor, an angiotensin converting enzyme inhibitor and a renin inhibitor.

In yet another example according to this aspect of the present invention, the pharmaceutical composition comprises a COX-2 inhibitor, a cathepsin inhibitor, an IGFR-1 pathway inhibitor and a renin inhibitor.

In yet another example according to this aspect of the present invention, the pharmaceutical composition comprises a COX-2 inhibitor, a cathepsin inhibitor, an angiotensin converting enzyme inhibitor and an IGFR-1 pathway inhibitor.

In yet another example according to this aspect of the present invention, the pharmaceutical composition comprises a COX-2 inhibitor, an angiotensin converting enzyme inhibitor, an IGFR-1 pathway inhibitor and a renin inhibitor.

In yet another example according to this aspect of the present invention, the pharmaceutical composition comprises a beta-blocker, a cathepsin inhibitor, an angiotensin converting enzyme inhibitor and a renin inhibitor.

In yet another example according to this aspect of the present invention, the pharmaceutical composition comprises a beta-blocker, a cathepsin inhibitor, an IFGR-1 pathway inhibitor and a renin inhibitor.

In yet another example according to this aspect of the present invention, the pharmaceutical composition comprises a beta-blocker, a cathepsin inhibitor, an angiotensin converting enzyme inhibitor and an IGFR-1 pathway inhibitor.

In yet another example according to this aspect of the present invention, the pharmaceutical composition comprises a beta-blocker, an angiotensin converting enzyme inhibitor, an IGFR-1 pathway inhibitor and a renin inhibitor.

In yet another example according to this aspect of the present invention, the pharmaceutical composition comprises a cathepsin inhibitor, an angiotensin converting enzyme inhibitor, an IGFR-1 pathway inhibitor and a renin inhibitor.

In a further example according to this aspect of the present invention, the pharmaceutical composition comprises a COX-2 inhibitor, a beta-blocker and a cathepsin inhibitor.

In a further example according to this aspect of the present invention, the pharmaceutical composition comprises a COX-2 inhibitor, a beta-blocker and a renin inhibitor.

In a further example according to this aspect of the present invention, the pharmaceutical composition comprises a COX-2 inhibitor, a beta-blocker and an angiotensin converting enzyme inhibitor.

In a further example according to this aspect of the present invention, the pharmaceutical composition comprises a COX-2 inhibitor, a beta-blocker, and an IGFR-1 pathway inhibitor.

In a further example according to this aspect of the present invention, the pharmaceutical composition comprises a COX-2 inhibitor, a cathepsin inhibitor and a renin inhibitor.

In a further example according to this aspect of the present invention, the pharmaceutical composition comprises a COX-2 inhibitor, a cathepsin inhibitor and an angiotensin converting enzyme inhibitor.

In a further example according to this aspect of the present invention, the pharmaceutical composition comprises a COX-2 inhibitor, a cathepsin inhibitor and an IGFR-1 pathway inhibitor.

In a further example according to this aspect of the present invention, the pharmaceutical composition comprises a COX-2 inhibitor, an angiotensin converting enzyme inhibitor and a renin inhibitor.

In a further example according to this aspect of the present invention, the pharmaceutical composition comprises a COX-2 inhibitor, an IGFR-1 pathway inhibitor and a renin inhibitor.

In a further example according to this aspect of the present invention, the pharmaceutical composition comprises a COX-2 inhibitor, an IGFR-1 pathway inhibitor, and an angiotensin converting enzyme inhibitor.

In a further example according to this aspect of the present invention, the pharmaceutical composition comprises a beta-blocker, a cathepsin inhibitor and a renin inhibitor.

In a further example according to this aspect of the present invention, the pharmaceutical composition comprises a beta-blocker, a cathepsin inhibitor and an angiotensin converting enzyme inhibitor.

In a further example according to this aspect of the present invention, the pharmaceutical composition comprises a beta-blocker, a cathepsin inhibitor and an IGFR-1 pathway inhibitor.

In a further example according to this aspect of the present invention, the pharmaceutical composition comprises a beta-blocker, an angiotensin converting enzyme inhibitor and a renin inhibitor.

In a further example according to this aspect of the present invention, the pharmaceutical composition comprises a beta-blocker, an IGFR-1 pathway inhibitor and a renin inhibitor.

In a further example according to this aspect of the present invention, the pharmaceutical composition comprises a beta-blocker, an IGFR-1 pathway inhibitor and an angiotensin converting enzyme inhibitor.

In a further example according to this aspect of the present invention, the pharmaceutical composition comprises a cathepsin inhibitor, an angiotensin converting enzyme inhibitor and a renin inhibitor.

In a further example according to this aspect of the present invention, the pharmaceutical composition comprises a cathepsin inhibitor, an IGFR-1 pathway inhibitor and a renin inhibitor.

In a further example according to this aspect of the present invention, the pharmaceutical composition comprises a cathepsin inhibitor, an IGFR-1 pathway inhibitor and an angiotensin converting enzyme inhibitor.

In a further example according to this aspect of the present invention, the pharmaceutical composition comprises an IGFR-1 pathway inhibitor, an angiotensin converting enzyme inhibitor and a renin inhibitor.

In yet a further example according to this aspect of the present invention, the pharmaceutical composition comprises a COX-2 inhibitor and a beta-blocker.

In yet a further example according to this aspect of the present invention, the pharmaceutical composition comprises a COX-2 inhibitor and a cathepsin inhibitor.

In yet a further example according to this aspect of the present invention, the pharmaceutical composition comprises a COX-2 inhibitor and a renin inhibitor.

In yet a further example according to this aspect of the present invention, the pharmaceutical composition comprises a COX-2 inhibitor and an angiotensin converting enzyme inhibitor.

In yet a further example according to this aspect of the present invention, the pharmaceutical composition comprises a COX-2 inhibitor and an IGFR-1 pathway inhibitor.

In yet a further example according to this aspect of the present invention, the pharmaceutical composition comprises a beta-blocker and a cathepsin inhibitor.

In yet a further example according to this aspect of the present invention, the pharmaceutical composition comprises a beta-blocker and a renin inhibitor.

In yet a further example according to this aspect of the present invention, the pharmaceutical composition comprises a beta-blocker and an angiotensin converting enzyme inhibitor.

In yet a further example according to this aspect of the present invention, the pharmaceutical composition comprises a beta-blocker and an IGFR-1 pathway inhibitor.

In yet a further example according to this aspect of the present invention, the pharmaceutical composition comprises a cathepsin inhibitor and a renin inhibitor.

In yet a further example according to this aspect of the present invention, the pharmaceutical composition comprises a cathepsin inhibitor and angiotensin converting enzyme inhibitor.

In yet a further example according to this aspect of the present invention, the pharmaceutical composition comprises a cathepsin inhibitor and an IGFR-1 pathway inhibitor.

In yet a further example according to this aspect of the present invention, the pharmaceutical composition comprises an angiotensin converting enzyme inhibitor and a renin inhibitor.

In yet a further example according to this aspect of the present invention, the pharmaceutical composition comprises an IGFR-1 pathway inhibitor and a renin inhibitor.

In yet a further example according to this aspect of the present invention, the pharmaceutical composition comprises an IGFR-1 pathway inhibitor and an angiotensin converting enzyme inhibitor.

It is possible that certain patients, when administered the drug combinations and pharmaceutical compositions according to the present invention, may develop an adverse reaction(s) or side effect(s) to certain angiotensin converting enzyme inhibitors such as Cilazapril or (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid (e.g.) troublesome coughing. Accordingly, it may be desirable to substitute the angiotensin converting enzyme inhibitor such as (e.g.) Cilazapril with an angiotensin receptor blocker (i.e. ATRB) such as (e.g.) Losartan or 2-butyl-4-chloro-1-{[2′-(1H-tetrazol-5-yl)biphenyl-4-yl]methyl}-1H-imidazol-5-yl)methanol. Suitable examples of other angiotensin receptor blockers (i.e. ATRB) in addition to Losartan include, but are not limited to, Irbesartan, Candesartan, Eprosartan, Olmesartan, Telmisartan, PD123319 and Valsartan.

Accordingly, in yet another aspect of the present invention there is provided a drug combination comprising a therapeutically effective amount of two or more compounds selected from a COX-2 inhibitor, a beta-blocker, a cathepsin inhibitor, an IGFR-1 pathway inhibitor, an angiotensin receptor blocker, a renin inhibitor, as well as combinations thereof.

In an example according to this aspect of the present invention, the drug combination comprises a COX-2 inhibitor, a beta-blocker, a cathepsin inhibitor, an IGFR-1 pathway inhibitor, an angiotensin receptor blocker and a renin inhibitor.

In another example according to this aspect of the present invention, the drug combination comprises a COX-2 inhibitor, a beta-blocker, a cathepsin inhibitor, an angiotensin receptor blocker and a renin inhibitor.

In another example according to this aspect of the present invention, the drug combination comprises a COX-2 inhibitor, a beta-blocker, an IGFR-1 pathway inhibitor, an angiotensin receptor blocker and a renin inhibitor.

In another example according to this aspect of the present invention, the drug combination comprises a COX-2 inhibitor, a cathepsin inhibitor, an IGFR-1 pathway inhibitor, an angiotensin receptor blocker and a renin inhibitor.

In another example according to this aspect of the present invention, the drug combination comprises a beta-blocker, a cathepsin inhibitor, an IGFR-1 pathway inhibitor, an angiotensin receptor blocker and a renin inhibitor.

In yet another example according to this aspect of the present invention, the drug combination comprises a COX-2 inhibitor, a beta-blocker, a cathepsin inhibitor and an angiotensin receptor blocker.

In yet another example according to this aspect of the present invention, the drug combination comprises a COX-2 inhibitor, a beta-blocker, an angiotensin receptor blocker and a renin inhibitor.

In yet another example according to this aspect of the present invention, the drug combination comprises a COX-2 inhibitor, a beta-blocker, an angiotensin receptor blocker and an IGFR-1 pathway inhibitor.

In yet another example according to this aspect of the present invention, the drug combination comprises a COX-2 inhibitor, a cathepsin inhibitor, an angiotensin receptor blocker and a renin inhibitor.

In yet another example according to this aspect of the present invention, the drug combination comprises a COX-2 inhibitor, a cathepsin inhibitor, an angiotensin receptor blocker and an IGFR-1 pathway inhibitor.

In yet another example according to this aspect of the present invention, the drug combination comprises a COX-2 inhibitor, an angiotensin receptor blocker, an IGFR-1 pathway inhibitor and a renin inhibitor.

In yet another example according to this aspect of the present invention, the drug combination comprises a beta-blocker, a cathepsin inhibitor, an angiotensin receptor blocker and a renin inhibitor.

In yet another example according to this aspect of the present invention, the drug combination comprises a beta-blocker, a cathepsin inhibitor, an angiotensin receptor blocker and an IGFR-1 pathway inhibitor.

In yet another example according to this aspect of the present invention, the drug combination comprises a beta-blocker, an angiotensin receptor blocker, an IGFR-1 pathway inhibitor and a renin inhibitor.

In yet another example according to this aspect of the present invention, the drug combination comprises a cathepsin inhibitor, an angiotensin receptor blocker, an IGFR-1 pathway inhibitor and a renin inhibitor.

In a further example according to this aspect of the present invention, the drug combination comprises a COX-2 inhibitor, a beta-blocker and an angiotensin receptor blocker.

In a further example according to this aspect of the present invention, the drug combination comprises a COX-2 inhibitor, a cathepsin inhibitor and an angiotensin receptor blocker.

In a further example according to this aspect of the present invention, the drug combination comprises a COX-2 inhibitor, an angiotensin receptor blocker and a renin inhibitor.

In a further example according to this aspect of the present invention, the drug combination comprises a COX-2 inhibitor, an IGFR-1 pathway inhibitor, and an angiotensin receptor blocker.

In a further example according to this aspect of the present invention, the drug combination comprises a beta-blocker, a cathepsin inhibitor and an angiotensin receptor blocker.

In a further example according to this aspect of the present invention, the drug combination comprises a beta-blocker, an angiotensin receptor blocker and a renin inhibitor.

In a further example according to this aspect of the present invention, the drug combination comprises a beta-blocker, an IGFR-1 pathway inhibitor and an angiotensin receptor blocker.

In a further example according to this aspect of the present invention, the drug combination comprises a cathepsin inhibitor, an angiotensin receptor blocker and a renin inhibitor.

In a further example according to this aspect of the present invention, the drug combination comprises a cathepsin inhibitor, an IGFR-1 pathway inhibitor and an angiotensin receptor blocker.

In a further example according to this aspect of the present invention, the drug combination comprises an IGFR-1 pathway inhibitor, an angiotensin receptor blocker and a renin inhibitor.

In yet a further example according to this aspect of the present invention, the drug combination comprises a COX-2 inhibitor and an angiotensin receptor blocker.

In yet a further example according to this aspect of the present invention, the drug combination comprises a beta-blocker and an angiotensin receptor blocker.

In yet a further example according to this aspect of the present invention, the drug combination comprises a cathepsin inhibitor and angiotensin receptor blocker.

In yet a further example according to this aspect of the present invention, the drug combination comprises an angiotensin receptor blocker and a renin inhibitor.

In yet a further example according to this aspect of the present invention, the drug combination comprises an IGFR-1 pathway inhibitor and an angiotensin receptor blocker.

As previously stated, the drug combinations according to the present invention may be formulated as one or more pharmaceutical compositions for simultaneous, separate and/or sequential administration to a patient in need thereof. By way of illustration only, where the drug combination comprises (e.g.) a COX-2 inhibitor and an angiotensin receptor blocker, the COX-2 inhibitor and angiotensin receptor blocker may be formulated as discrete pharmaceutical compositions for separate and/or sequential administration to a patient in need thereof, or in the same pharmaceutical composition for simultaneous administration to a patient in need thereof. However, formulation of the COX-2 inhibitor and the angiotensin receptor blocker in separate pharmaceutical compositions does not preclude simulataneous administration to a patient.

Accordingly, in another aspect of the present invention there is provided a pharmaceutical composition comprising a therapeutically effective amount of two or more compounds selected from a COX-2 inhibitor, a beta-blocker, a cathepsin inhibitor, an IGFR-1 pathway inhibitor, an angiotensin receptor blocker, a renin inhibitor, as well as combinations thereof, together with a pharmaceutically effective carrier.

In an example according to this aspect of the present invention, the pharmaceutical composition comprises a COX-2 inhibitor, a beta-blocker, a cathepsin inhibitor, an IGFR-1 pathway inhibitor, an angiotensin receptor blocker and a renin inhibitor.

In another example according to this aspect of the present invention, the pharmaceutical composition comprises a COX-2 inhibitor, a beta-blocker, a cathepsin inhibitor, an angiotensin receptor blocker and a renin inhibitor.

In another example according to this aspect of the present invention, the pharmaceutical composition comprises a COX-2 inhibitor, a beta-blocker, an IGFR-1 pathway inhibitor, an angiotensin receptor blocker and a renin inhibitor.

In another example according to this aspect of the present invention, the pharmaceutical composition comprises a COX-2 inhibitor, a cathepsin inhibitor, an IGFR-1 pathway inhibitor, an angiotensin receptor blocker and a renin inhibitor.

In another example according to this aspect of the present invention, the pharmaceutical composition comprises a beta-blocker, a cathepsin inhibitor, an IGFR-1 pathway inhibitor, an angiotensin receptor blocker and a renin inhibitor.

In yet another example according to this aspect of the present invention, the pharmaceutical composition comprises a COX-2 inhibitor, a beta-blocker, a cathepsin inhibitor and an angiotensin receptor blocker.

In yet another example according to this aspect of the present invention, the pharmaceutical composition comprises a COX-2 inhibitor, a beta-blocker, an angiotensin receptor blocker and a renin inhibitor.

In yet another example according to this aspect of the present invention, the pharmaceutical composition comprises a COX-2 inhibitor, a beta-blocker, an angiotensin receptor blocker and an IGFR-1 pathway inhibitor.

In yet another example according to this aspect of the present invention, the pharmaceutical composition comprises a COX-2 inhibitor, a cathepsin inhibitor, an angiotensin receptor blocker and a renin inhibitor.

In yet another example according to this aspect of the present invention, the pharmaceutical composition comprises a COX-2 inhibitor, a cathepsin inhibitor, an angiotensin receptor blocker and an IGFR-1 pathway inhibitor.

In yet another example according to this aspect of the present invention, the pharmaceutical composition comprises a COX-2 inhibitor, an angiotensin receptor blocker, an IGFR-1 pathway inhibitor and a renin inhibitor.

In yet another example according to this aspect of the present invention, the pharmaceutical composition comprises a beta-blocker, a cathepsin inhibitor, an angiotensin receptor blocker and a renin inhibitor.

In yet another example according to this aspect of the present invention, the pharmaceutical composition comprises a beta-blocker, a cathepsin inhibitor, an angiotensin receptor blocker and an IGFR-1 pathway inhibitor.

In yet another example according to this aspect of the present invention, the pharmaceutical composition comprises a beta-blocker, an angiotensin receptor blocker, an IGFR-1 pathway inhibitor and a renin inhibitor.

In yet another example according to this aspect of the present invention, the pharmaceutical composition comprises a cathepsin inhibitor, an angiotensin receptor blocker, an IGFR-1 pathway inhibitor and a renin inhibitor.

In a further example according to this aspect of the present invention, the pharmaceutical composition comprises a COX-2 inhibitor, a beta-blocker and an angiotensin receptor blocker.

In a further example according to this aspect of the present invention, the pharmaceutical composition comprises a COX-2 inhibitor, a cathepsin inhibitor and an angiotensin receptor blocker.

In a further example according to this aspect of the present invention, the pharmaceutical composition comprises a COX-2 inhibitor, an angiotensin receptor blocker and a renin inhibitor.

In a further example according to this aspect of the present invention, the pharmaceutical composition comprises a COX-2 inhibitor, an IGFR-1 pathway inhibitor, and an angiotensin receptor blocker.

In a further example according to this aspect of the present invention, the pharmaceutical composition comprises a beta-blocker, a cathepsin inhibitor and an angiotensin receptor blocker.

In a further example according to this aspect of the present invention, the pharmaceutical composition comprises a beta-blocker, an angiotensin receptor blocker and a renin inhibitor.

In a further example according to this aspect of the present invention, the pharmaceutical composition comprises a beta-blocker, an IGFR-1 pathway inhibitor and an angiotensin receptor blocker.

In a further example according to this aspect of the present invention, the pharmaceutical composition comprises a cathepsin inhibitor, an angiotensin receptor blocker and a renin inhibitor.

In a further example according to this aspect of the present invention, the pharmaceutical composition comprises a cathepsin inhibitor, an IGFR-1 pathway inhibitor and an angiotensin receptor blocker.

In a further example according to this aspect of the present invention, the pharmaceutical composition comprises an IGFR-1 pathway inhibitor, an angiotensin receptor blocker and a renin inhibitor.

In yet a further example according to this aspect of the present invention, the pharmaceutical composition comprises a COX-2 inhibitor and an angiotensin receptor blocker.

In yet a further example according to this aspect of the present invention, the pharmaceutical composition comprises a beta-blocker and an angiotensin receptor blocker.

In yet a further example according to this aspect of the present invention, the pharmaceutical composition comprises a cathepsin inhibitor and angiotensin receptor blocker.

In yet a further example according to this aspect of the present invention, the pharmaceutical composition comprises an angiotensin receptor blocker and a renin inhibitor.

In yet a further example according to this aspect of the present invention, the pharmaceutical composition comprises an IGFR-1 pathway inhibitor and an angiotensin receptor blocker.

According to the drug combinations, compositions and formulations of the present invention, examples of cyclo-oxygenase 2 inhibitors (referred to herein as COX-2 inhibitors) includes, but is not limited to, Celecoxib, Nepafenac, Ibuprofen (Dolgesic), Indomethacin, Sulindac, Xanthohumol, Meclofenamate Sodium, Meloxicam, Rofecoxib, Bromfenac Sodium, Ibuprofen Lysine, Ketorolac (Ketorolac tromethamine), Diclofenac Sodium, Etodolac, Ketoprofen, Naproxen Sodium, Piroxicam, Acemetacin, Phenacetin, Tolfenamic Acid, Nimesulide, Flunixin Meglumin, Aspirin, Bufexamac, Niflumic acid, Licofelone, Oxaprozin, Lornoxicam, Lumiracoxib, Zaltoprofen, Ampiroxicam, Valdecoxib, Nabumetone, Mefenamic Acid, Carprofen, Amfenac Sodium monohydrate, Asaraldehyde and Suprofen and includes non-steroidal anti-inflammatory drugs (NSAIDs).

According to the drug combinations, compositions and formulations of the present invention, examples of non-steroidal anti-inflammatory drugs (also referred to herein as a “NSAID”) includes, but is not limited to, Salicylates, including, but not limited to, Salicyclic Acid, Acetylsalicylic Acid, Salsalate, Diflunisal; Propionic Acid derivatives, including, but not limited to, Ibuprofen, Dexibuprofen, Naproxen, Denoprofen, Ketoprofen, Dexketoprofen, Flubirpofen, Oxaprozin and loxoprofen; Acetic Acid derivatives, including, but not limited to, Indoemthacin, Tolmetin, Sulindac, Etodolac, Ketorolac, Diclofenac, Aceclofenac, Nabumetone; Enolic Acid (Oxicam) derivatives, including, but not limited to, Piroxicam, Meloxicam, Tenoxicam, Droxicam, Lornoxicam, Isoxicam and Phenylbutazone; Anthranilic Acid derivatives, including, but not limited to, Mefenamic Acid, Meclofenamic Acid, Flufenamic Acid, Tolfenamic Acid; COX-2 Inhibitors, including, but not limited to, Celecoxib, Rofecoxib, Valdecoxib, Parecoxib, Lumiracoxib, Etoricoxib; Sulfonamides, including, but not limited to, Nimesulide; Clonixin; and Licofelone.

Also according to the drug combinations, compositions and formulations of the present invention, examples of beta-blockers (also referred to herein as a “p-blocker” or “β-blockers”) includes, but is not limited to, Acebutolol (Sectral), Atenolol (Tenormin), Betaxolol (Betoptic), Bisoprolol (Cardicor, Emcor, Zebeta), Carteolol (Teoptic), Carvedilol (Coreg, Eucardic), Celiprolol (Celectol), Labetalol (Trandate), Levobunolol (Betagan), Metipranolol (Metipranolol Minims), Metoprolol (Betaloc, Lopresor, Lopressor, Toprol XL), Nadolol (Corgard), Nebivolol (Bystolic, Nebilet), Oxprenolol (Trasicor), Pindolol (Visken), Propranolol (Inderal LA), Sotalol (Beta-Cardone, Sotacor), and Timolol (Betim, Nyogel, Timoptol).

Also according to the drug combinations, compositions and formulations of the present invention, examples of cathepsin inhibitors include cathepsin B and cathepsin D inhibitors. Examples of cathepsin B inhibitors includes, but is not limited to, Curcumin, Cystatin B, Cystatin C, Cysteine peptidase inhibitor E64, [Pt(dmba)(aza-N1)(dmso)] complex 1 (a potential anti-tumoral drug with lower IC50 than cisplatin in several tumoral cell lines), 2,3,7,8-Tetrachlorodibenzo-p-dioxin (TCDD), CA-074Me, Lipidated CtsB inhibitor incorporated into the envelope of a liposomal nanocarrier (LNC-NS-629), Proanthocyanidin (PA) and Ahpatinin Ac (1) and Ahpatinin Pr (2). Examples of cathepsin D inhibitors includes, but is not limited to, non-peptidic acylguanidine inhibitors of Cathepsin D, Pepstatin A, Bm-Aspin, SIPI, Via, RNAi-Rab27A and Solanum lycopersicum aspartic protease inhibitor (SLAPI).

Also according to the drug combinations, compositions and formulations of the present invention, examples of angiotensin converting enzyme inhibitors (also referred to herein as “ACE inhibitor”, ACE inhibitors” or “ACEi”) includes, but is not limited to, Benazepril (Lotesin), Captopril (Capoten), Cilazipril, Enalapril (Vasotec, Renitec), Fosinopril (Monopril), Lisinopril (Lisodur, Lopril, Novatec, Prinivil, Zestril), Moexipril, Perindopril (Coversay, Aceon), Quinapril (Accupril), Ramipril (Altace, Tritace, Ramace, Ramiwin), Trandolapril, Delapril, Zofenopril and Imidapril.

Also according to the drug combinations, compositions and formulations of the present invention, examples of IGFR-1 pathway inhibitor is selected from metformin, tyrphostins such as AG538 and AG1024, pyrrolo(2,3-d)-pyrimidine derivatives such as NVP-AEW541 and Figitumumab (also called CP-751871).

Also according to the drug combinations, compositions and formulations of the present invention, examples of renin inhibitor (also referred to herein as “direct renin inhibitor(s)”) Aliskiren.

Also according to the drug combinations, compositions and formulations of the present invention, examples of angiotensin receptor blockers include, but are not limited to, Losartan, Irbesartan, Candesartan, Eprosartan, Olmesartan, Telmisartan, PD123319 and Valsartan.

In another aspect of the present invention there is provided a drug combination or a pharmaceutical composition comprising Aspirin, Propanolol and Curcumin. In a related aspect, the drug combination or a pharmaceutical composition comprises acetylsalicylic acid, (RS)-1-(1-methylethylamino)-3-(1-naphthyloxy)propan-2-ol and (1E,6E)-1,7-Bis(4-hydroxy-3-methoxyphenyl)hepta-1,6-diene-3,5-dione.

In a further aspect of the present invention there is provided a drug combination or pharmaceutical composition comprising Aspirin, Curcumin and Aliskiren. In a related aspect, the drug combination or a pharmaceutical composition comprises acetylsalicylic acid, (1E,6E)-1,7-Bis(4-hydroxy-3-methoxyphenyl)hepta-1,6-diene-3,5-dione and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide.

In yet a further aspect of the present invention there is provided a drug combination or a pharmaceutical composition comprising Celecoxib, Propanalol and Curcumin. In a related aspect, the drug combination or pharmaceutical composition comprises 4-[5-(4-methylphenyl)-3-(trifluoromethyl)pyrazol-1-yl]benzenesulfonamide, (RS)-1-(1-methylethylamino)-3-(1-naphthyloxy)propan-2-ol and (1E,6E)-1,7-Bis(4-hydroxy-3-methoxyphenyl)hepta-1,6-diene-3,5-dione.

In yet a further aspect of the present invention there is provided a drug combination or a pharmaceutical composition comprising Celecoxib, Curcumin and Aliskiren. In a related aspect, the drug combination or pharmaceutical composition comprises 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide.

In yet another aspect of the present invention there is provided a drug combination or a pharmaceutical composition comprising Curcumin, Propanolol, Aspirin and Quinapril. In a related aspect, the drug combination or pharmaceutical composition comprises (1E,6E)-1,7-Bis(4-hydroxy-3-methoxyphenyl)hepta-1,6-diene-3,5-dione, (RS)-1-(1-methylethylamino)-3-(1-naphthyloxy)propan-2-ol, acetylsalicylic acid and [3S-[2[R*(R)],3R*]]-2-[2-[[1-Ethoxycarbonyl)-3-phenylpropyl]amino]-1-oxopropyl]-1,2,3,4-tetrahydro-3-isoquinolinecarboxylic acid monohydrochloride.

In a further aspect of the present invention there is provided a drug combination or pharmaceutical composition comprising Aliskiren, Celecoxib and Curcumin. In a related aspect the drug combination or pharmaceutical composition comprises (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide and (1E,6E)-1,7-Bis(4-hydroxy-3-methoxyphenyl)hepta-1,6-diene-3,5-dione.

In yet a further aspect of the present invention there is provided a drug combination or pharmaceutical composition comprising Aliskiren, Celecoxib, Curcumin and Metformin. In a related aspect the drug combination or pharmaceutical composition comprises (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione and N,N-dimethylimidodicarbonimidic diamide.

In yet a further aspect of the present invention there is provided a drug combination or pharmaceutical composition comprising Aliskiren, Celecoxib, Curcumin, Metformin and Propanolol. In a related aspect the drug combination or pharmaceutical composition comprises (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide and (RS)-1-(1-methylethylamino)-3-(1-naphthyloxy)propan-2-ol.

In yet a further aspect of the present invention there is provided a drug combination or pharmaceutical composition comprising Aliskiren, Celecoxib, Curcumin, Metformin, Propanolol and Cilazapril. In a related aspect the drug combination or pharmaceutical composition comprises (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide, (RS)-1-(1-methylethylamino)-3-(1-naphthyloxy)propan-2-ol and (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid.

Further still, where the combinations, compositions and formulations according to the present invention comprise Curcumin, the combinations, compositions and formulations may further comprise an agent that increases the bioavailability of Curcumin. Examples of agents that increase the bioavailability of curcumin includes, but is not limited to, 1-[5-(1,3-Benzodioxol-5-yl)-1-oxo-2,4-pentadienyl]piperidine, extract from piper nigrum (black pepper), bromelain (protease enzyme from pineapple stems).

As stated previously, it is possible that certain patients may develop an adverse reaction to an angiotensin converting enzyme inhibitor (e.g.) Cilazapril or (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid (e.g.) troublesome coughing. Accordingly, it may be desirable to substitute the angiotensin converting enzyme inhibitor (e.g.) Cilazapril with an angiotensin receptor blocker (i.e. ATRB; refer to FIG. 1) such as (e.g.) Losartan or 2-butyl-4-chloro-1-{[2′-(1H-tetrazol-5-yl)biphenyl-4-yl]methyl}-1H-imidazol-5-yl)methanol.

Accordingly, in a further aspect of the present invention there is provided a drug combination or pharmaceutical composition comprising:
(i) Aspirin, Propanolol, Curcumin and Piperidine;
(ii) Aspirin, Curcumin, Aliskiren and Piperidine;
(iii) Celecoxib, Propanolol, Curcumin and Piperidine;
(iv) Curcumin, Propanolol, Aspirin, Quinapril and Piperidine;
(v) Aliskiren, Celecoxib, Curcumin and Piperidine;
(vi) Aliskiren, Celecoxib, Curcumin, Metformin and Piperidine;
(vii) Aliskiren, Celecoxib, Curcumin, Metformin, Propanolol and Piperidine;
(viii) Aliskiren, Celecoxib, Curcumin, Metformin, Propanolol, Cilazapril and Piperidine;
(ix) Aliskiren, Celecoxib, Curcumin, Metformin, Propanolol, Losartan; and
(x) Aliskiren, Celecoxib, Curcumin, Metformin, Propanolol, Losartan and Piperidine.

In a related aspect, the drug combination or pharmaceutical compisition comprises:
(i) acetylsalicylic acid, (RS)-1-(1-methylethylamino)-3-(1-naphthyloxy)propan-2-ol, (1E,6E)-1,7-Bis(4-hydroxy-3-methoxyphenyl)hepta-1,6-diene-3,5-dione and 1-[5-(1,3-Benzodioxol-5-yl)-1-oxo-2,4-pentadienyl]piperidine;
(ii) acetylsalicylic acid, (1E,6E)-1,7-Bis(4-hydroxy-3-methoxyphenyl)hepta-1,6-diene-3,5-dione, (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide and 1-[5-(1,3-Benzodioxol-5-yl)-1-oxo-2,4-pentadienyl]piperidine;
(iii) 4-[5-(4-methylphenyl)-3-(trifluoromethyl)pyrazol-1-yl]benzenesulfonamide, (RS)-1-(1-methylethylamino)-3-(1-naphthyloxy)propan-2-ol, (1E,6E)-1,7-Bis(4-hydroxy-3-methoxyphenyl)hepta-1,6-diene-3,5-dione and 1-[5-(1,3-Benzodioxol-5-yl)-1-oxo-2,4-pentadienyl]piperidine;
(iv) (1E,6E)-1,7-Bis(4-hydroxy-3-methoxyphenyl)hepta-1,6-diene-3,5-dione, (RS)-1-(1-methylethylamino)-3-(1-naphthyloxy)propan-2-ol, acetylsalicylic acid, [3S-[2[R*(R)],3R*]]-2-[2-[[1-Ethoxycarbonyl)-3-phenylpropyl]amino]-1-oxopropyl]-1,2,3,4-tetrahydro-3-isoquinolinecarboxyiic acid monohydrochloride and 1-[5-(1,3-Benzodioxol-5-yl)-1-oxo-2,4-pentadienyl]piperidine;
(v) (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione and 1-[5-(1,3-Benzodioxol-5-yl)-1-oxo-2,4-pentadienyl]piperidine;
(vi) (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide and 1-[5-(1,3-Benzodioxol-5-yl)-1-oxo-2,4-pentadienyl]piperidine;
(vii) (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide, (RS)-1-(1-methylethylamino)-3-(1-naphthyloxy)propan-2-ol and 1-[5-(1,3-Benzodioxol-5-yl)-1-oxo-2,4-pentadienyl]piperidine; and
(viii) (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide, (RS)-1-(1-methylethylamino)-3-(1-naphthyloxy)propan-2-ol, (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid and 1-[5-(1,3-Benzodioxol-5-yl)-1-oxo-2,4-pentadienyl]piperidine;
(ix) (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide, (RS)-1-(1-methylethylamino)-3-(1-naphthyloxy)propan-2-ol and 2-butyl-4-chloro-1-{[2′-(1H-tetrazol-5-yl)biphenyl-4-yl]methyl}-1H-imidazol-5-yl)methanol; and (x) (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide, (RS)-1-(1-methylethylamino)-3-(1-naphthyloxy)propan-2-ol, 2-butyl-4-chloro-1-{[2′-(1H-tetrazol-5-yl)biphenyl-4-yl]methyl}-1H-imidazol-5-yl)methanol and 1-[5-(1,3-Benzodioxol-5-yl)-1-oxo-2,4-pentadienyl]piperidine.

In certain examples according to the methods and compositions described herein, the cancer patient being treated may already been on a course of an anti-hypertensive drug, such as (e.g.) thiazide diuretics, calcium channel blockers, ACE inhibitors, angiotensin receptor blockers (ATRBs), and beta blockers. As such, the combinations, compositions and formulations of the present invention do not need to include, for example, an angiotensin converting enzyme inhibitor or a beta-blocker.

Accordingly, in yet a further aspect of the present invention there is provided a drug combination or a pharmaceutical composition comprising Aspirin, Propanolol, Curcumin, Metformin and Aliskiren or a drug combination or a pharmaceutical composition comprising Aspirin, Curcumin, Metformin, Cilazapril and Aliskiren or a drug combination or a pharmaceutical composition comprising Aspirin, Curcumin, Metformin and Aliskiren. In a related aspect, the drug combination or a pharmaceutical composition comprises acetylsalicylic acid, (RS)-1-(1-methylethylamino)-3-(1-naphthyloxy)propan-2-ol, (1E,6E)-1,7-Bis(4-hydroxy-3-methoxyphenyl)hepta-1,6-diene-3,5-dione, N,N-dimethylimidodicarbonimidic diamide and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, or a drug combination or a pharmaceutical composition comprising acetylsalicylic acid, (1E,6E)-1,7-Bis(4-hydroxy-3-methoxyphenyl)hepta-1,6-diene-3,5-dione, N,N-dimethylimidodicarbonimidic diamide, (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, or a drug combination or a pharmaceutical composition comprising acetylsalicylic acid, (1E,6E)-1,7-Bis(4-hydroxy-3-methoxyphenyl)hepta-1,6-diene-3,5-dione, N,N-dimethylimidodicarbonimidic diamide and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide.

In yet another aspect of the present invention there is provided a drug combination or a pharmaceutical composition comprising Celecoxib, Propanolol, Curcumin, Metformin and Aliskiren or a drug combination or a pharmaceutical composition comprising Celecoxib, Curcumin, Metformin, Cilazapril and Aliskiren or a drug combination or a pharmaceutical composition comprising Celecoxib, Curcumin, Metformin and Aliskiren. In a related aspect, the drug combination or a pharmaceutical composition comprises 4-[5-(4-methylphenyl)-3-(trifluoromethyl)pyrazol-1-yl]benzenesulfonamide, (RS)-1-(1-methylethylamino)-3-(1-naphthyloxy)propan-2-ol, (1E,6E)-1,7-Bis(4-hydroxy-3-methoxyphenyl)hepta-1,6-diene-3,5-dione, N,N-dimethylimidodicarbonimidic diamide and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, or a drug combination or a pharmaceutical composition comprising 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide, (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, or a drug combination or a pharmaceutical composition comprising 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide.

In yet a further aspect of the present invention there is provided a drug combination or a pharmaceutical composition comprising Aspirin and Curcumin or Celecoxib and Curcumin. In a related example, the drug combination or a pharmaceutical composition comprises acetylsalicylic acid and (1E,6E)-1,7-Bis(4-hydroxy-3-methoxyphenyl)hepta-1,6-diene-3,5-dione or 4-[5-(4-methylphenyl)-3-(trifluoromethyl)pyrazol-1-yl]benzenesulfonamide and (1E,6E)-1,7-Bis(4-hydroxy-3-methoxyphenyl)hepta-1,6-diene-3,5-dione. In yet another aspect of the present invention there is provided a drug combination or pharmaceutical composition comprising Aspirin, Curcumin and Cilazapril or Celecoxib, Curcumin and Cilazapril. In a related example, the drug combination or a pharmaceutical composition comprises acetylsalicylic acid, (1E,6E)-1,7-Bis(4-hydroxy-3-methoxyphenyl)hepta-1,6-diene-3,5-dione and (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid or 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione and (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid.

Again, in certain situations or for certain patients it may be advantagous for the drug combinations and pharmaceutical compositions referred to in the paragraphs immediately above (i.e. as related to patients already on a course of an anti-hypertensive drug, such as (e.g.) thiazide diuretics, calcium channel blockers, ACE inhibitors, angiotensin receptor blockers (ATRBs), and beta blockers) to substitute Cilazapril for Losartan, and/or include (e.g.) Piperidine to increase the bioavailability of Curcumin. The present invention explicitly contemplates such drug combinations and pharmaceutical compisitions.

Where the various drug combinations and/or pharmaceutical compositions referred to throughout this specification comprise a non-steroidal anti-inflammatory agent, (e.g.) acetylsalicylic acid, the combination or compositions may further comprise an agent that reduces the likelihood of peptic ulcers in the stomach. An example of an agent that reduces the likelihood of peptic ulcers in the stomach includes, but is not limited to, 5-methoxy-2-[(4-methoxy-3,5-dimethylpyridin-2-yl)methylsulfinyl]-1H-benzimidazole (ie. Omeprazole). In an example according to the present invention, the amount of Omeprazole administered to a patient comprises up to 20 mg per patient per day. Again, the present invention explicitly contemplates such drug combinations and pharmaceutical compisitions.

Further, the various drug combinations and/or pharmaceutical compositions may additionally comprise an agent that increases the bioavailability of Curcumin or (1E,6E)-1,7-Bis(4-hydroxy-3-methoxyphenyl)hepta-1,6-diene-3,5-dione. An example of an agent that increases the bioavailability of (1E,6E)-1,7-Bis(4-hydroxy-3-methoxyphenyl)hepta-1,6-diene-3,5-dione includes, but is not limited to, 1-[5-(1,3-Benzodioxol-5-yl)-1-oxo-2,4-pentadienyl]piperidine (ie. Piperidine). The combination of Curcumin and Piperidine (e.g.) ‘Doctors Best High Absorption Curcumin with C3 Complex and BioPerine, 1000 mg from iHerb could be used, refer to http:/nz.iherb.com/Doctor-s-Best-High-Absortion-Curcurmin-with-C3-Complex-and-BioPerine-1-000-m-120-Tablets/12137.

In further examples, the drug combinations according to the present invention may be adapted for simultanous, separate or sequential administration to a patient in need thereof. In an example, where the drug combination according to the present invention is adapted for simultaneous administration, the drug combination may be administered in the same pharmaceutical composition or in separate pharmaceutical compositions administered simultaneously and optionally in combination with another pharmaceutically active agent.

The present invention also contemplates different routes of administration for the combinations, compositions and formulations according to the present invention. Examples of administration routes include, but are not limited to, oral, transdermal delivery, topical application, suppository delivery, transmucosal delivery, injection (including subcutaneous administration, subdermal administration, intramuscular administration, depot administration, and intravenous administration, including delivery via bolus, slow intravenous injection, and intravenous drip), infusion devices (including implantable infusion devices, both active and passive), administration by inhalation or insufflation, buccal administration and sublingual administration. In a preferred example according to the present invention, administration is via the oral route. The different modes of administration are provided in further detail below.

According to the drug combinations, compositions and formulations, in certain examples of the present invention:
(i) (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide is administered to a patient in need thereof via the oral route up to a maximum daily amount of 300 mg;
(ii) 4-[5-(4-methylphenyl)-3-(trifluoromethyl)pyrazol-1-yl]benzenesulfonamide is administered to a patient in need thereof via the oral route up to a maximum daily amount of 200 mg;
(iii) (1E,6E)-1,7-Bis(4-hydroxy-3-methoxyphenyl)hepta-1,6-diene-3,5-dione is administered to a patient in need thereof via the oral route up to a maximum daily amount of 1000 mg;
(iv) N,N-dimethylimidodicarbonimidic diamide is administered to a patient in need thereof via the oral route up to a maximum daily amount of 1000 mg;
(v) (RS)-1-(1-methylethylamino)-3-(1-naphthyloxy)propan-2-ol is administered to a patient in need thereof via the oral route up to a maximum daily amount of 160 mg;
(vi) (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid is administered to a patient in need thereof via the oral route up to a maximum daily amount of 5 mg; and
(vii) 2-butyl-4-chloro-1-{[2′-(1H-tetrazol-5-yl)biphenyl-4-yl]methyl}-1H-imidazol-5-yl)methanol is administered to a patient in need thereof via the oral route up to a maximum daily amount of 100 mg.

A person skilled in the art would recognise the term “up to [amount of drug] mg” means that amount of drug may be administered to a patient in the stipulated period (e.g. total daily dose or maximum tolerated dose (per day)) or a lesser amount. For example, a daily dose of up to 200 mg means that a single dose of 200 mg may be administered to the patient in any given 24 h period, or a single dose in an amount that is (e.g.) 175 mg, 150 mg, 125 mg, 100 mg or less may be administered to the patient in any given 24 h period. Further, a daily dose of “up to [amount of drug] mg” might mean that the drug is administered in several doses (e.g. twice or three times daily) provided that the total amount of drug administered to the patient does not exceed the maximum amount stipulated. Following this teaching, and using the example above, a daily dose of up to 200 mg might mean two discrete doses of 100 mg is administered to the patient at different time points in any given 24 h period, or three discrete doses of 66.6 mg is administerd to the patient at three different time points in any given 24 h period.

Since the present invention contemplates administration of many different drugs from varied drug classes (e.g. refer to the Drug Combination Example 6), in certain examples it is deirable to administer drug formulations that are said to be “long acting” or allow for “sustained release” of the active compound over a period of time (e.g. several hours to a week or month). To illustrate this point, Example 5 outlines a clinical trial which involves administration of the beta-blocker propanolol. At different time points in the dosing regime, a single dose of a formulation comprising up to 160 mg of propanolol is used, which formulation is said to be long acting. As such, it is only necessary to administer this formulation of propanolol to a patient once per day, which reduced the total number of administrations required.

In other examples according to the combinations, compositions and formulations, in certain examples of the present invention:
(i) acetylsalicylic acid is administered to a patient in need thereof via the oral route up to a maximum daily amount of 300 mg or 4-[5-(4-methylphenyl)-3-(trifluoromethyl)pyrazol-1-yl]benzenesulfonamide is administered to the patient via the oral route up to a daily maxiumum amount of 400 mg;
(ii) optionally, 5-methoxy-2-[(4-methoxy-3,5-dimethylpyridin-2-yl)methylsulfinyl]-1H-benzimidazole is administered to a patient in need thereof via the oral route up to a maximum daily amount of 20 mg;
(iii) (RS)-1-(1-methylethylamino)-3-(1-naphthyloxy)propan-2-ol is administered to a patient in need thereof via the oral route up to a maximum daily amount of 320 mg;
(iv) (1E,6E)-1,7-Bis(4-hydroxy-3-methoxyphenyl)hepta-1,6-diene-3,5-dione is administered to a patient in need thereof via the oral route up to a maximum daily amount of 8000 mg;
(v) optionally, 1-[5-(1,3-Benzodioxol-5-yl)-1-oxo-2,4-pentadienyl]piperidine is administered to a patient in need thereof via the oral route, where the ratio of (1E,6E)-1,7-Bis(4-hydroxy-3-methoxyphenyl)hepta-1,6-diene-3,5-dione to 1-[5-(1,3-Benzodioxol-5-yl)-1-oxo-2,4-pentadienyl]piperidine is between 1000:20, and in particular 500:20;
(vi) N,N-dimethylimidodicarbonimidic diamide is administered to a patient in need thereof via the oral route up to a maximum daily amount of 2500 mg;
(vii) (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid is administered to a patient in need thereof via the oral route up to a maximum daily amount of 5 mg;
(viii) (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide is administered to a patient in need thereof via the oral route up to a maximum daily amount of 300 mg.

The drug combinations, compositions and formulations according to the present invention are useful in the prevention, treatment and/or management of cancer and non-cancerous tumours, including the recurrence of cancer. In particular, Example 1 documents treatment of a patient having Stage IV Adenocarcinoma of the Lung, which cancer has a very poor short-term prognosis. By using a treatment regime comprising drug combinations or pharmaceutical compositions that target or modulate the Renin-Angiotensin System (RAS) in accordance with the teaching of the present invention, the patient's disease pathology and outlook significantly improved. Further, use of the drug combinations, pharmaceutical compositions, methods and treatment regimes described herein, improved the disease pathology and overall health of a patient having Endometrioma (Example 2) and a separate patient having Throat Cancer (Example 3). Further, patients having oral cavity squamous cell carcinoma (OCSCC), locally advanced and/or metastatic head and neck skin squamous cell carcinoma (HNsSCC), glioblastoma multiforme (GBM) and malignant melanoma (MM) have now been recruited for a clinical trial (Example 4) and administered treatment regimes comprising the drug combinations, pharmaceutical compositions and articles of manufacture described and exemplified herein.

Disease symptoms improved, and in some cases, a reduction in tumour growth was observed.

Accordingly, in yet a further aspect of the present invention there is provided a method for preventing, treating and/or managing cancer or a non-cancerous tumour in a patient in need thereof, the method comprising administering to the patient a prophylactically or therapeutically effective amount of one or more drug combinations or a pharmaceutical compositions as described herein.

In a related aspect of the present invention there is provided a drug combination or a pharmaceutical composition as described herein, for use in preventing, treating and/or managing cancer or a non-cancerous tumour in a patient.

In a further related aspect of the present invention there is provided use of a drug combination or a pharmaceutical composition as descibed herein, in the manufacture of a medicament for preventing, treating and/or managing cancer or a non-cancerous tumour in a patient in need thereof.

In an example according to the above aspects of the present invention, the cancer is selected from squamous cell carcinoma of the upper aerodigestive tract (including oral cavity), squamous cell carcinoma of the skin, melanoma, lung cancer, breast cancer, kidney cancer, brain cancer, bowel cancer, thyroid cancer, prostate cancer, lymphoma, leukemia and sarcomas. In particular examples according to the above aspects of the present invention, the cancer is selected from oral cavity squamous cell carcinoma (OCSCC), recurrent locally advanced and/or metastatic head and neck cutaneous squamous cell carcinoma (HNcSCC), recurrent malignant melanoma (MM) and recurrent glioblastoma multiforme (GBM).

In another aspect of the present invention, there is provided a method for preventing, treating and/or managing cancer or a non-cancerous tumour in a patient in need thereof, the method comprising the steps of administering to the patient:
(i) a drug combination or a pharmaceutical composition comprising (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide and (1E,6E)-1,7-Bis(4-hydroxy-3-methoxyphenyl)hepta-1,6-diene-3,5-dione via an oral route of administration for a period of about two weeks; and
(ii) a drug combination or a pharmaceutical composition comprising (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione and N,N-dimethylimidodicarbonimidic diamide via an oral route of administration for a period of about another two weeks to about another four weeks;
(iii) a drug combination or a pharmaceutical composition comprising (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide and (RS)-1-(1-methylethylamino)-3-(1-naphthyloxy)propan-2-ol via an oral route of administration for a period of about a further two weeks to about a further four weeks; and
(iv) a drug combination or a pharmaceutical composition comprising (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, 4-[5-(4-methylphenyl)-3-(trifluoromethyl)pyrazol-1-yl]benzenesulfonamide, (1E,6E)-1,7-Bis(4-hydroxy-3-methoxyphenyl)hepta-1,6-diene-3,5-dione, N,N-dimethylimidodicarbonimidic diamide, (RS)-1-(1-methylethylamino)-3-(1-naphthyloxy)propan-2-ol and (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid via an oral route of administration for a period of about another two weeks to about another six weeks or longer, as required.

In certain examples, according to this aspect of the present invention:
(i) (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide is formulated for oral administration to a patient in a total daily amount of between up to 150 mg and up to 300 mg;
(ii) 4-[5-(4-methylphenyl)-3-(trifluoromethyl)pyrazol-1-yl]benzenesulfonamide is formulated for oral administration to a patient in a total daily amount of up to 200 mg;
(iii) (1E,6E)-1,7-Bis(4-hydroxy-3-methoxyphenyl)hepta-1,6-diene-3,5-dione is formulated for oral administration to a patient in a total daily amount of between up to 500 mg and up to 1000 mg;
(iv) N,N-dimethylimidodicarbonimidic diamide is formulated for oral administration to a patient in a total daily amount of between 500 mg and 1000 mg;
(v) (RS)-1-(1-methylethylamino)-3-(1-naphthyloxy)propan-2-ol diamide is formulated for oral administration to a patient in a total daily amount of between up tp 80 mg and up to 160 mg; and
(vi) (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid is formulated for oral administration to a patient in a total daily amount of between up to 1.25 mg and up to 5 mg.

In other examples according to this aspect of the present invention:
(i) step (i) of the method described herein comprises administering a maximum daily amount of up to 150 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, a total daily amount of up to 200 mg 4-[5-(4-methylphenyl)-3-(trifluoromethyl)pyrazol-1-yl]benzenesulfonamid, and a total daily amount of up to 100 mg of (1E,6E)-1,7-Bis(4-hydroxy-3-methoxyphenyl)hepta-1,6-diene-3,5-dione, to the patient for a period of about two weeks;
(ii) step (ii) of the method described herein comprises administering a maximum daily amount of up to 300 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, a total daily amount of up to 200 mg 4-[5-(4-methylphenyl)-3-(trifluoromethyl)pyrazol-1-yl]benzenesulfonamid, a total daily amount of up to 100 mg of (1E,6E)-1,7-Bis(4-hydroxy-3-methoxyphenyl)hepta-1,6-diene-3,5-dione and a total daily amount of up to 500 mg of N,N-dimethylimidodicarbonimidic diamide, to the patient for an initial period of about two weeks;
(iii) step (ii) of the method described herein further comprises administering a maximum daily amount of up to 300 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, a total daily amount of up to 200 mg 4-[5-(4-methylphenyl)-3-(trifluoromethyl)pyrazol-1-yl]benzenesulfonamid, a total daily amount of up to 100 mg of (1E,6E)-1,7-Bis(4-hydroxy-3-methoxyphenyl)hepta-1,6-diene-3,5-dione and a total daily amount of up to 1000 mg of N,N-dimethylimidodicarbonimidic diamide, to the patient for a subsequent period of about two weeks;
(iv) step (iii) of the method described herein comprises administering a maximum daily amount of up to 300 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, a total daily amount of up to 200 mg 4-[5-(4-methylphenyl)-3-(trifluoromethyl)pyrazol-1-yl]benzenesulfonamid, a total daily amount of up to 100 mg of (1E,6E)-1,7-Bis(4-hydroxy-3-methoxyphenyl)hepta-1,6-diene-3,5-dione, a total daily amount of up to 1000 mg of N,N-dimethylimidodicarbonimidic diamide and a total daily amount of up to about 80 mg of (RS)-1-(1-methylethylamino)-3-(1-naphthyloxy)propan-2-ol, to the patient for an initial period of about two weeks;
(v) step (iii) of the method described herein further comprises administering a maximum daily amount of up to 300 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, a total daily amount of up to 200 mg 4-[5-(4-methylphenyl)-3-(trifluoromethyl)pyrazol-1-yl]benzenesulfonamid, a total daily amount of up to 100 mg of (1E,6E)-1,7-Bis(4-hydroxy-3-methoxyphenyl)hepta-1,6-diene-3,5-dione, a total daily amount of up to 1000 mg of N,N-dimethylimidodicarbonimidic diamide and a total daily amount of up to about 160 mg of (RS)-1-(1-methylethylamino)-3-(1-naphthyloxy)propan-2-ol, to the patient for a subsequent period of about two weeks;
(vi) step (iv) of the method described herein comprises administering a maximum daily amount of up to 300 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, a total daily amount of up to 200 mg 4-[5-(4-methylphenyl)-3-(trifluoromethyl)pyrazol-1-yl]benzenesulfonamid, a total daily amount of up to 100 mg of (1E,6E)-1,7-Bis(4-hydroxy-3-methoxyphenyl)hepta-1,6-diene-3,5-dione, a total daily amount of up to 1000 mg of N,N-dimethylimidodicarbonimidic diamide, a total daily amount of up to about 160 mg of (RS)-1-(1-methylethylamino)-3-(1-naphthyloxy)propan-2-ol and a total daily amount of up to 1.25 mg of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid, to the patient for an initial period of about two weeks;
(vii) step (iv) of the method described herein further comprises administering a maximum daily amount of up to 300 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, a total daily amount of up to 200 mg 4-[5-(4-methylphenyl)-3-(trifluoromethyl)pyrazol-1-yl]benzenesulfonamid, a total daily amount of up to 100 mg of (1E,6E)-1,7-Bis(4-hydroxy-3-methoxyphenyl)hepta-1,6-diene-3,5-dione, a total daily amount of up to 1000 mg of N,N-dimethylimidodicarbonimidic diamide, a total daily amount of up to about 160 mg of (RS)-1-(1-methylethylamino)-3-(1-naphthyloxy)propan-2-ol and a total daily amount of up to 2.5 mg of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid, to the patient for a subsequent period of about two weeks; and
(viii) step (iv) of the method described herein further comprises administering a maximum daily amount of up to 300 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, a total daily amount of up to 200 mg 4-[5-(4-methylphenyl)-3-(trifluoromethyl)pyrazol-1-yl]benzenesulfonamid, a total daily amount of up to 100 mg of (1E,6E)-1,7-Bis(4-hydroxy-3-methoxyphenyl)hepta-1,6-diene-3,5-dione, a total daily amount of up to 1000 mg of N,N-dimethylimidodicarbonimidic diamide, a total daily amount of up to about 160 mg of (RS)-1-(1-methylethylamino)-3-(1-naphthyloxy)propan-2-ol and a total daily amount of up to 5 mg of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid, to the patient for yet a further period of about two weeks or more.

In yet another aspect of the present invention there is provided a method for preventing, treating and/or managing cancer in a patient, the method comprising the steps of:
(i) where the patient is taking an angiotensin converting enzyme inhibitor other than (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid then changing to an equivalent dose of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid by administering 1.25 mg, 2.5 mg or 5.0 mg of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid to the patient once per day; and
(ii) administering 300 mg of acetylsalicylic acid to the patient once per day;
(iii) administering 150 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide to the patient once per day; and
(iv) administering 500 mg (1E,6E)-1,7-Bis(4-hydroxy-3-methoxyphenyl)hepta-1,6-diene-3,5-dione to the patient twice per day;

wherein the (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid and/or acetylsalicylic acid, (1E,6E)-1,7-Bis(4-hydroxy-3-methoxyphenyl)hepta-1,6-diene-3,5-dione and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide are administered to the patient in the stated doses simultaneously, separately or sequentially in accordance with steps (i), (ii), (iii) and (iv) for a first dose period of about two weeks; and (v) at the conclusion of the first dose period, increasing the amount of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide by administering 150 mg of (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide to the patient twice per day; and
(vi) administering 500 mg of N,N-dimethylimidodicarbonimidic diamide to the patient once per day;

wherein the (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid and/or acetylsalicylic acid, (1E,6E)-1,7-Bis(4-hydroxy-3-methoxyphenyl)hepta-1,6-diene-3,5-dione, (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide and N,N-dimethylimidodicarbonimidic diamide is administered to the patient in the stated doses simultaneously, separately or sequentially in accordance with steps in accordance with steps (i), (ii), (iv), (v) and (vi) for a second dose period of about two weeks; and
(vii) at the conclusion of the second dose period, increasing the amount of N,N-dimethylimidodicarbonimidic diamide by administering 500 mg of N,N-dimethylimidodicarbonimidic diamide to the patient twice per day; and
(viii) administering 40 mg of (RS)-1-(1-methylethylamino)-3-(1-naphthyloxy)propan-2-ol to the patient twice per day;

wherein the (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid and/or acetylsalicylic acid, (1E,6E)-1,7-Bis(4-hydroxy-3-methoxyphenyl)hepta-1,6-diene-3,5-dione, (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, N,N-dimethylimidodicarbonimidic diamide and (RS)-1-(1-methylethylamino)-3-(1-naphthyloxy)propan-2-ol is administered to the patient in the stated doses simultaneously, separately or sequentially in accordance with steps in accordance with steps (i), (ii), (iv), (v), (vii) and (viii) for a third dose period of about two weeks; and
(ix) at the conclusion of the third dose period, increasing the amount of (RS)-1-(1-methylethylamino)-3-(1-naphthyloxy)propan-2-ol by administering 40 mg of (RS)-1-(1-methylethylamino)-3-(1-naphthyloxy)propan-2-ol to the patient three times per day,

wherein the (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid and/or acetylsalicylic acid, (1E,6E)-1,7-Bis(4-hydroxy-3-methoxyphenyl)hepta-1,6-diene-3,5-dione, (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, N,N-dimethylimidodicarbonimidic diamide and (RS)-1-(1-methylethylamino)-3-(1-naphthyloxy)propan-2-ol is administered to the patient in the stated doses simultaneously, separately or sequentially in accordance with steps (i), (ii), (iv), (v), (vii) and (ix) for a fourth dose period of about two weeks; and
(x) at the conclusion of the fourth dose period, where the patient is already taking (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid increasing the amount of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid by administering 5.0 mg of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid to the patient once per day or where the patient has not been taking (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid, administering 2.5 mg of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid to the patient once per day;

wherein the (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid and/or acetylsalicylic acid, (1E,6E)-1,7-Bis(4-hydroxy-3-methoxyphenyl)hepta-1,6-diene-3,5-dione, (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, N,N-dimethylimidodicarbonimidic diamide and (RS)-1-(1-methylethylamino)-3-(1-naphthyloxy)propan-2-ol is administered to the patient in the stated doses simultaneously, separately or sequentially in accordance with steps (ii), (iv), (v), (vii), (ix) and (x) for a fifth dose period of about two weeks; and
(xi) at the conclusion of the fifth dose period increasing the amount of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid by administering 5.0 mg of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid to the patient once per day where the patient was previously being administered only 2.5 mg of (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid once per day.

In certain examples according to the above methods and treatment regimes, the patient may already be taking medication (e.g.) anti-hypertension medication. In this example, the anti-hypertension medication may include one or more of a beta-blocker and/or an angiotensin converting enzyme inhibitor. As such, where the patient is already taking a course of medication that is identical or equivalent to one or more drugs described in the methods and treatment regimes according to the present invention, the skilled person would appreciate that the drug combinations, pharmaceutical compositions, methods and treatment regimes can be modified to take account of existing therapies.

In an example according to these aspects of the present invention, the acetylsalicylic acid, (RS)-1-(1-methylethylamino)-3-(1-naphthyloxy)propan-2-ol, (1E,6E)-1,7-Bis(4-hydroxy-3-methoxyphenyl)hepta-1,6-diene-3,5-dione, N,N-dimethylimidodicarbonimidic diamide, (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid and (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide are administered orally.

In yet another aspect of the present invention there is provided an article of manufacture comprising one or more of the drug combinations or pharmaceutical compositions as described herein, and optionally instructions for how to prevent, treat and/or manage cancer or a non-cancerous tumour in a patient in need thereof.

In yet another aspect of the present invention there is provided a kit comprising one or more of the drug combinations or pharmaceutical compositions as described herein, and optionally instructions for how to prevent, treat and/or manage cancer or a non-cancerous tumour in a patient in need thereof.

The combinations, compositions and formulations according to the present invention are also useful in the prevention, treatment and/or management of non-cancerous tumours including benign tumours.

### Renin-Angiotensin System

The Renin-Angiotensin System (RAS) is traditionally known to preserve fluid volume during periods of restricted dietary salt and also prevents ischaemia during acute volume loss. The main effector peptide of the RAS is angiotensin II (ATII). It induces vasoconstriction and sympathetic activation, raises aldosterone levels, and promotes renal salt and water retention via the angiotensin II receptor 1 (ATIIR1). Over the last few decades, the RAS has been a drug target of particular interest because of its involvement in cardiovascular disease (CVD) and renovascular disease. The CVD and renovascular disease can be understood as a continuum of risk factors, target organ damage, events, and mortality. Risk factors (such as hypertension, dyslipidemia, diabetes, and smoking) led to the development of target organ damage including atherosclerosis, left ventricular hypertrophy (LVH), and renal impairment. Target organ damage progressively worsens, leading ultimately to myocardial infarction (MI), heart failure (HF), end-stage renal disease (ESRD), stroke, or death.

ATII, the main effector peptide of the RAS, plays an active role during all stages of this continuum. The first step in the RAS cascade is the formation of angiotensin I (ATI) from the precursor angiotensinogen under the action of renin; early evidence for the importance of RAS in CVD came from the consistent finding that renin activity is predictive of the risk of cardiovascular (CV) events. ATI is then converted to ATII, the principal effector peptide of the RAS, by angiotensin-converting enzyme (ACE). In addition, ATII can be produced in tissues by enzymes such as chymase. This locally produced ATII is believed to mediate paracrine and autocrine functions. ATII acts via ATIIR1 and ATIIR2. Activation of ATIIR1 results in vasoconstriction, aldosterone and vasopressin secretion, sodium retention, and decreased renal perfusion. Hence, these receptors mediate the deleterious effects of ATII, including elevated blood pressure (BP) and cardiac and vascular remodelling. The effects of the ATII receptors have been less clearly defined because of the limited expression of these receptors in adults, because of their unconventional signalling pathways, and because many ATII-mediated actions are masked by opposing ATI-mediated effects. However, it is now recognised that ATIIR2 generally opposes the actions of ATIIR1, mediating various anti-proliferative and anti-inflammatory effects and promoting tissue differentiation and regeneration and apoptosis.

Additional components of the RAS have been identified in the last decade, including bioactive angiotensin peptides, such as angiotensin III, angiotensin IV, and angiotensin-(1-7), the effects of which have not yet been fully elucidated for the CV and renal system.

The discovery of the renin receptor has shed further light on the biology of the RAS. Renin, simply considered until recently as the rate-limiting enzyme of RAS activation, has also turned out to be the ligand for a protein known as the renin/prorenin receptor that binds renin and prorenin about equally, regardless of their biologic activities. Prorenin, which represents 70% to 90% of total circulating renin, when bound to the receptor induces an increase in the catalytic efficiency of angiotensinogen conversion to ATI, which contributes to the local production of ATII and its systemic levels, as well as binding of renin/prorenin to the renin/prorenin receptor, exerting physiologic effects that are independent of ATII, including activation of intracellular signal pathways, enhanced synthesis of DNA, and stimulation of the release of plasminogen activator inhibitor 1, collagen 1, fibronectin, and transforming growth factor 3-1.6 There are a number of known drugs which target the RAS. The two major classes of drugs that target the RAS are the angiotensin-converting enzyme (ACE) inhibitors and the angiotensin receptor blockers (ARBs). Although both of these drug classes target ATII, the differences in their mechanisms of action have implications for their effects on other pathways and receptors that may have therapeutic implications. Both ACEIs and ARBs are effective antihypertensive agents that have been shown to reduce the risk of cardiovascular and renal events.

Direct inhibition of renin, the most proximal aspect of the RAS, became clinically feasible from 2007 with the introduction of Aliskiren. This latter drug has been shown to be efficacious for the management of hypertension. Combined therapy of direct renin-inhibitors with ACEIs or ARBs has been tested in some clinical situations such as congestive heart failure (HF) and proteinuria with diverse results.

RAS drugs include, but are not limited to, Angiotensin-Converting Enzyme Inhibitors (ACEIs), Angiotensin Receptor Blockers (ARBs), Direct Renin Inhibitors (DRIs), Beta-Blockers, Cyclo-oxygenase 2 Inhibitors, Chymase Inhibitors, Cathepsin Inhibitors including Cathepsin Inhibitors, Cathepsin D Inhibitors and Cathepsin G Inhibitors, Calcium Channel Blockers, Calcium Supplements and Vitamin D, as described above.

### Cancer Therapy Agents

The methods and therapeutic regimes described herein for the treatment and/or management of a cancer, e.g., oral cavity squamous cell carcinoma (OCSCC), recurrent locally advanced and/or metastatic head and neck cutaneous SCC (HNcSCC), recurrent malignant melanoma (MM) and recurrent glioblastoma multiforme (GBM), comprise administration of various drug combinations comprising therapeutically effective agents that target or modulate the Renin-Angiotensin System (RAS). Examples of therapeutically effective agents, which form part of a drug combinations, pharmaceutical compositions and formulations that target or modulate RAS of the present invention, include, but are not limited to, COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. Specific examples of therapeutically effective agents which fall within the definition of these drug classes have been listed elsewhere in this specification and are incorporated herein by reference. In accordance with the present invention, the RAS modulating drug combinations (including compositions, pharmaceutical compositions, formulations, articles of manufacture and kits) may be administered in series or in combination with (e.g., in physical combination, provided as a combined preparation) with one or more other cancer therapy agents.

Accordingly, also contemplated within the scope of the present invention is the selection of other inhibitors/pharmaceutically active molecules that target the Renin-Angiotensin System (RAS) expressed by cancer stem/cells, for use in the drug combinations and/or pharmaceutical compositions described herein. Examples include, but are not limited to, angiotensin receptor blockers, cyclo-oxygenase 2 inhibitors, inhibitors of cathepsin D, inhibitors of cathepsin G, calcium channel blockers, calcium supplements and vitamin D.

Examples of angiotensin receptor blockers include, but are not limited to, Losartan, Irbesartan, Candesartan, Eprosartan, Olmesartan, Telmisartan, PD123319 and Valsartan.

Examples of inhibitors of cathepsin D include, but are not limited to, non-peptidic acylguanidine inhibitors of Cathepsin D, Pepstatin A, Bm-Aspin, SIPI, Via, RNAi-Rab27A and Solanum lycopersicum aspartic protease inhibitor (SLAPI).

Examples of inhibitors of cathepsin G include, but are not limited to, WFDC12, Phenylmethylsulfonyl fluoride (PMSF), Ecotin, SerpinB1, SerpinA3, CeEI, or Caesalpinia echinata elastase inhibitor, SLPI (secretory leukocyte protease inhibitor), Alpha1-Antitrypsin (AAT), Bauhinia bauhinoides cruzipain inhibitor, Alpha-Aminoalkylphosphonate diaryl esters, Greglin, [2-[3-[[(1-benzoyl-4-piperidinyl)methylamino]carbonyl]-2-naphthalenyl]-1-(1-naphthalenyl)-2-oxoethyl]-phosphonic acid (KPA), Lympho-Epithelial Kazal-Type-related Inhibitor (LEKTI), Trappin-2 A62L, SV-66, SCGI, Bortezomib, Human monocyte/neutrophil elastase inhibitor (MNEI), a 42-kDa serpin protein and Anti-leukoproteinase (ALP).

Examples of calcium channel blockers include, but are not limited to, dihydropyridine calcium channel blockers, phenylalkylamine calcium channel blockers, benzothiazepine calcium channel blockers, non-selective calcium channel blockers.

Examples of dihydropyridine calcium channel blockers include, but are not limted to, Amlodipine (Norvasc), Aranidipine (Sapresta), Azelnidipine (Calblock), Barnidipine (HypoCa), Benidipine (Coniel), Cilnidipine (Atelec, Cinalong, Siscard), Clevidipine (Cleviprex), Isradipine (DynaCirc, Prescal), Efonidipine (Landel), Felodipine (Plendil), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas).

Examples of phenylalkylamine calcium channel blockers include, but are not limited to, Verapamil (Calan, Isoptin), Gallopamil and Fendiline.

Examples of benzothiazepine calcium channel blockers include, but are not limited to, Diltiazem (Cardizem) and Fendiline.

Examples of non-selective calcium channel blockers include, but are not limited to, Mibefradil, Bepridil, Flunarizine, Fluspirilene and Fendiline.

Examples of other calcium channel blockers include, but are not limited to, Gabapentin, Pregabalin and Ziconotide.

In the methods of the present invention for the prevention and/or treatment of a cancer, e.g., squamous cell carcinoma of the upper aerodigestive tract (including oral cavity), squamous cell carcinoma of the skin, melanoma, lung cancer, breast cancer, kidney cancer, brain cancer, bowel cancer, thyroid cancer, prostate cancer, lymphoma, leukemia and sarcomas, sub-therapeutically effective amounts of RAS modulating drug combinations as described herein, and one or more other cancer therapy agents are used or provided for combined administration (separately or jointly as a combined preparation) to provide a combined action that is therapeutically effective.

Treatment of a subject or patient with the combinations, compositions or formulations as described herein may comprise their acute or sustained administration and, in the case of combinations, their simultaneous, separate, or sequential administration, as further described herein.

The combinations, compositions or formulations of the present invention may be administered to a subject in need of treatment, such as a subject with any of the diseases, disorders or conditions mentioned herein. The condition of the subject can thus be improved. The agents may be used in the manufacture of a medicament to treat any of the diseases, disorders or condtions mentioned herein. Thus, in accordance with the invention, there are provided formulations by which cancers can be treated.

A therapeutically effective amount of each of the combinations of therapeutically active agents (e.g., COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors) may be administered simultaneously, separately or sequentially and in any order. The therapeutically active agents may be administered separately or as a fixed combination. When not administered as a fixed combination, preferred methods include the sequential administration of the therapeutically active agents, either or both of which are provided in amounts or doses that are less than those used when the drug or drugs are administered alone, i.e., when they are not administered in combination, either physically or in the course of treatment. Such lesser amounts of drugs administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone. Preferably, the agents are administered sequentially within at least about one-half hour of each other. The agents may also be administered within about one hour of each other, within about one day to about one week of each other, or as otherwise deemed appropriate.

### Dosage Forms and Formulations and Administration

The therapeutically active agents administered as part of the combinations, compositions or formulations according to the present invention may be present in an isolated or substantially or essentially pure form. It will be understood that the combinations, compositions or formulations may be mixed with carriers or diluents which will not interfere with the intended purpose of the product and still be regarded as isolated or substantially pure. A product of the invention may also be in a substantially or essentialy purified form, preferably comprising or consisting essentially of about 80%, 85%, or 90%, e.g. at least about 95%, at least about 98% or at least about 99% of the compound or dry mass of the preparation.

Depending on the intended route of administration, the combinations, compositions or formulations including medicaments of the invention may, for example, take the form of solutions, suspensions, instillations, sustained release formulations, or powders, and typically contain about 0.1%-95% of active ingredient(s), preferably about 0.2%-70%. Other suitable formulations include injection- and infusion-based formulations. Other useful formulations include sustained release preparations, including, for example, controlled, slow or delayed release preparations.

Aspects of the present invention include controlled or other doses, dosage forms, formulations, compositions and/or devices containing two or therapeutically active agents, wherein the therapeutically active agents are, for example, COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. The present invention includes, for example, doses and dosage forms for at least oral administration, transdermal delivery, topical application, suppository delivery, transmucosal delivery, injection (including subcutaneous administration, subdermal administration, intramuscular administration, depot administration, and intravenous administration, including delivery via bolus, slow intravenous injection, and intravenous drip), infusion devices (including implantable infusion devices, both active and passive), administration by inhalation or insufflation, buccal administration and sublingual administration. It will be appreciated that any of the dosage forms, compositions, formulations or devices described herein particularly for intravenous administration may be utilised, where applicable or desirable, in a dosage form, composition, formulation or device for administration by any of the other routes herein contemplated or commonly employed. For example, a dose or doses could be given parenterally using a dosage form suitable for parenteral administration which may incorporate features or compositions described in respect of dosage forms suitable for oral administration, or be delivered in an sustained dosage form, such as a modified release, extended release, delayed release, slow release or repeat action dosage form.

In certain examples according to the present invention, the therapeutically active agents of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or pH buffering agents may also be present.

The term “pharmaceutically acceptable carrier” refers to any useful carriers, excipients, or stabilizers which are nontoxic to the cell or mammal being exposed thereto at the dosages and concentrations employed, and include pharmaceutical carriers that do not induce the production of antibodies harmful to the individual receiving the composition. Suitable carriers can be large, slowly metabolized macromolecules such as proteins, polysaccharides, polylactic acids, polyglycolic acids, polymeric amino acids, and amino acid copolymers. Often the physiologically acceptable carrier is an aqueous pH buffered solution. Other examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, polyethylene glycol (PEG), and Pluronics.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, gels, emulsions, or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

An auxiliary agent such as casein, gelatin, albumin, glue, sodium alginate, carboxymethylcellulose, methylcellulose, hydroxyethylcellulose or polyvinyl alcohol may also be included in the formulation of the invention.

The dosage forms, combinations, compositions, formulations and/or devices of the invention may be formulated to optimize bioavailability and to maintain plasma concentrations within the therapeutic range, including for extended periods. Sustained delivery preparations, e.g., controlled delivery preparations, also optimize the drug concentration at the site of action and minimize periods of under and over medication, for example.

The dosage forms, devices and/or compositions useful in the invention may be provided for periodic administration, including once daily administration, for low dose controlled and/or low dose long-lasting in vivo release of (e.g.) COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors.

Examples of dosage forms suitable for transdermal administration include, but are not limited to, transdermal patches, transdermal bandages, and the like. Examples of dosage forms suitable for topical administration of the compounds and formulations useful in the invention are any lotion, stick, spray, ointment, paste, cream, gel, etc., whether applied directly to the skin or via an intermed.

Examples of dosage forms suitable for suppository administration of the compounds and formulations useful in the invention include any solid dosage form inserted into a bodily orifice particularly those inserted rectally, vaginally and urethrally.

Examples of dosage forms suitable for transmucosal delivery of the compounds and formulations useful in the invention include depositories solutions for enemas, pessaries, tampons, creams, gels, pastes, foams, nebulised solutions, powders and similar formulations containing in addition to the active ingredients such carriers as are known in the art to be appropriate.

Examples of dosage of forms suitable for injection of the compounds and formulations useful in the invention include delivery via bolus such as single or multiple administrations by intravenous injection, subcutaneous, subdermal, and intramuscular administration or oral administration.

Examples of dosage forms suitable for depot administration of the compounds and formulations useful in the invention include pellets or small cylinders of active agent or solid forms wherein the active agent is entrapped in a matrix of biodegradable polymers, microemulsions, liposomes or is microencapsulated.

Examples of infusion devices for compounds and formulations useful in the invention include infusion pumps containing one or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, at a desired amount for a desired number of doses or steady state administration, and include implantable drug pumps.

Examples of implantable infusion devices for compounds and formulations useful in the invention include any solid form in which the active agent is encapsulated within or dispersed throughout a biodegradable polymer or synthetic, polymer such as silicone, silicone rubber, silastic or similar polymer.

Examples of dosage forms suitable for inhalation or insufflation of compounds and formulations useful in the invention include compositions comprising solutions and/or suspensions in pharmaceutically acceptable, aqueous, or organic solvents, or mixture thereof and/or powders.

Examples of dosage forms suitable for buccal administration of the compounds and formulations useful in the invention include lozenges, tablets and the like, compositions comprising solutions and/or suspensions in pharmaceutically acceptable, aqueous, or organic solvents, or mixtures thereof and/or powders.

Examples of dosage forms suitable for sublingual administration of the compounds and formulations useful in the invention include lozenges, tablets and the like, compositions comprising solutions and/or suspensions in pharmaceutically acceptable, aqueous, or organic solvents, or mixtures thereof and/or powders.

Examples of controlled drug formulations for delivery of the compounds and formulations useful in the invention are found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville MD, 1995 (hereinafter “the USP”), which also describes specific tests to determine the drug release capabilities of extended-release and delayed-release tablets and capsules. Further guidance concerning the analysis of extended release dosage forms has been provided by the FDA. See Guidance for Industry. Extended release oral dosage forms: development, evaluation, and application of in vitro/in vivo correlations. Rockville, MD: Center for Drug Evaluation and Research, Food and Drug Administration (1997).

Further examples of dosage forms useful in the methods of the invention include, but are not limited to, modified-release (MR) dosage forms including delayed-release (DR) forms; prolonged-action (PA) forms; controlled-release (CR) forms; extended-release (ER) forms; timed-release (TR) forms; and long-acting (LA) forms. For the most part, these terms are used to describe orally administered dosage forms, however these terms may be applicable to any of the dosage forms, formulations, compositions and/or devices described herein. These formulations effect delayed total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

Modified-release dosage forms of the invention include dosage forms having drug release features based on time, course, and/or location which are designed to accomplish therapeutic or convenience objectives not offered by conventional or immediate-release forms. See, for example, Bogner, R. H. U.S. Pharmacist 22 (Suppl.):3-12 (1997); Scale-up of oral extended-release drug delivery systems: part I, an overview, Pharmaceutical Manufacturing 2:23-27 (1985). Extended-release dosage forms of the invention include, for example, as defined by The United States Food and Drug Administration (FDA), a dosage form that allows a reduction in dosing frequency to that presented by a conventional dosage form, e.g., a solution or an immediate-release dosage form. See, for example, Bogner, R. H. (1997) supra. Repeat action dosage forms of the invention include, for example, forms that contain two single doses of medication, one for immediate release and the second for delayed release. Bi-layered tablets, for example, may be prepared with one layer of drug for immediate release with the second layer designed to release drug later as either a second dose or in an extended-release manner. Targeted-release dosage forms of the invention include, for example, formulations that facilitate drug release and which are directed towards isolating or concentrating a drug in a body region, tissue, or site for absorption or for drug action.

Also useful in the invention are coated beads, granules or microspheres containing one or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, which may be used to achieve modified release of one or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors by incorporation of the drug into coated beads, granules, or microspheres. In such systems, the one or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors are distributed onto beads, pellets, granules or other particulate systems. See Ansel, H. C., Allen, L. V. and Popovich, N. G., Pharmaceutical Dosage Forms and Drug Delivery Systems, 7th Ed., Lippincott 1999, p. 232.

Methods for manufacture of microspheres suitable for drug delivery have been described. See, for example, Arshady, R. Polymer Eng Sci 30:1746-1758 (1989); see also, Arshady, R., Polymer Eng Sci 30:905-914 (1990); see also: Arshady R., Polymer Eng Sci 30:915-924 (1990). Various coating systems are commercially available. E.g., Aquacoat™ [FMC Corporation, Philadelphia] and Surerelease™ [Colorcon]; Aquacoat aqueous polymeric dispersion. Philadelphia: FMC Corporation, 1991; Surerelease aqueous controlled release coating system. West Point, PA: Colorcon, 1990; Butler, J., et al., Pharm Tech 22:122-138 (1998); Yazici, E., et al., Pharmaceut Dev Technol 1:175-183 (1996).

Variation in the thickness of the coats and in the type of coating materials used affects the rate at which the body fluids are capable of penetrating the coating to dissolve the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. Generally, the thicker the coat, the more resistant to penetration and the more delayed release and dissolution of the therapeutic agents. See Madan, P. L. U.S. Pharmacist 15:39-50 (1990). This provides the different desired sustained or extended release rates and the targeting of the coated beads to the desired segments of the gastrointestinal tract. Examples of film-forming polymers which can be used in water-insoluble release-slowing intermediate layer(s) (to be applied to a pellet, spheroid or tablet core) include ethylcellulose, polyvinyl acetate, Eudragit® RS, Eudragit® RL, etc. Each of Eudragit® RS and Eudragit® RL is an ammonio methacrylate copolymer. The release rate can be controlled not only by incorporating therein suitable water-soluble pore formers, such as lactose, mannitol, sorbitol, etc., but also by the thickness of the coating layer applied. Multi-tablets may be formulated which include small spheroid-shaped compressed mini-tablets that may have a diameter of between 3 to 4 mm and can be placed in a gelatin capsule shell to provide the desired pattern of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors release. Each capsule may contain 8-10 minitablets, some uncoated for immediate release and others coated for extended release of the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors.

A number of methods may be employed to generate modified-release dosage forms of one or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors suitable for oral administration to humans and other mammals. Two basic mechanisms available to achieve modified release drug delivery include altered dissolution or diffusion of drugs and excipients. Within this context, for example, four processes may be employed, either simultaneously or consecutively. These are as follows: (i) hydration of the device (e.g., swelling of the matrix); (ii) diffusion of water into the device; (iii) controlled or delayed dissolution of the drug; and (iv) controlled or delayed diffusion of dissolved or solubilized drug out of the device.

In order to formulate a range of dosage values, cell culture assays and animal studies can be used. The dosage of such compounds preferably lies within the dose that is therapeutically effective for at least 50% of the population, and that exhibits little or no toxicity at this level.

The effective dosage of each of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors employed in the methods and compositions of the invention may vary depending on a number of factors including the particular COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors employed, the cancer therapeutic combinational partner if present, the mode of administration, the frequency of administration, the condition being treated, the severity of the condition being treated, the route of administration, the needs of a patient sub-population to be treated or the needs of the individual patient whose different needs can be due to age, sex, body weight, relevant medical condition specific to the patient.

A suitable dose may be from about 0.001 to about 1 or to about 10 mg/kg body weight such as about 0.01 to about 0.5 mg/kg body weight. A suitable dose may however be from about 0.001 to about 0.1 mg/kg body weight such as about 0.01 to about 0.05 mg/kg body weight. Doses from about 1 to 100, 100-200, 200-300, 300-400, and 400-500 miligrams are appropriate, as are doses of about 500-750 micrograms and about 750-1000 micrograms. Other useful doses include from about 300 to about 1000 picomoles per dose, and about 0.05 to about 0.2 nanomoles per dose. Still other doses are within the following claims.

For example, in certain examples, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors composition may be administered at about 0.01 nanomolar (mM) or 0.05 nM to about 200 nM final concentration. Preferably, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors composition is administered at about 0.1 nM to about 150 nM final concentration, more preferably, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors composition is applied at about 1 nM to about 100 nM final concentration, and more preferably, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors composition is administered at about 10-20 nM to about 100-150 nM final concentration. Additionally, COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors dose amounts include, for example, about 0.1-1, 1-2, 2-3, 3-4, or 4-5 milligrams (mg), from about 5 to about 10 mg, from about 10 to about 15 mg, from about 15 to about 20 mg, from about 20 to about 30 mg, from about 30 to about 40 mg, from about 40 to about 50 mg, from about 50 to about 75 mg, from about 75 to about 100 mg, from about 100 mg to about 250 mg, and from 250 mg to about 500 mg. Dose amounts from 500 to about 1000 and from 1000 to about 2000 milligrams or more or also provided, as noted above.

Still other dosage levels between about 1 nanogram (ng)/kg and about 1 mg/kg body weight per day of each of the agents described herein. In certain examples, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 0.01 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg/kg body weight, about 0.01 mg to about 0.1 mg/kg body weight, about 0.1 mg to about 1 mg/kg body weight. Where more than one COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors is used, the dosage of each COX-2 inhibitor including non-steroidal anti-inflammatory drugs, beta-blockers, inhibitors of the IGFR-1 pathway, inhibitors of cathepsin, angiotensin converting enzyme inhibitors and (direct) renin inhibitors need not be in the same range as the other.

Conveniently, if infused, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors are administered for at least about 0.5 to 1 hour, at least about 1-2 hours, at least about 2-4 hours, at least about 4-6 hours, at least about 6-8 hours, at least about 8-10 hours, at least about 12 hours, or at least about 24 hours.

As noted herein, the doses of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, for example, administered in combination, or other cancer therapeutic agents administered in combination with either or both, can be adjusted down from the doses administered when given alone.

The combined use of several agents may reduce the required dosage for any individual agent because the onset and duration of effect of the different agents may be complementary. In a preferred example, the combined use of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors and/or cancer therapeutic agents has an additive, synergistic or super-additive effect.

In some cases, the combination of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors and cancer therapeutic agent, or other agents administered in combination with either or both, has an additive effect. In other cases, the combination can have greater-than-additive effect. Such an effect is referred to herein as a “supra-additive” effect, and may be due to synergistic or potentiated interaction.

In another preferred example, the combined use of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors and another cancer therapeutic agent, reduces the frequency in which said agent is administered compared to the frequency when said agent is administered alone. Thus, these combinations allow the use of lower and/or fewer doses of each agent than previously required to achieve desired therapeutic goals.

Doses may be administered in single or divided applications. The doses may be administered once, or the application may be repeated. Typically, administration can be by infusion in addition to or instead of multiple single adminstrations.

One or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, combinations thereof and optionally inclusive of another cancer therapeutic agent, if desired, may be administered by the same or different routes. The various agents of the invention can be administered separately at different times during the course of therapy, or concurrently in divided or single combination forms.

In one example of the invention a COX-2 inhibitor including non-steroidal anti-inflammatory drugs, beta-blockers, inhibitors of the IGFR-1 pathway, inhibitors of cathepsin, angiotensin converting enzyme inhibitors and (direct) renin inhibitors is administered in one composition and another cancer therapeutic agent (including a COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors) is administered in a second composition. In another example the first composition comprising COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors is administered before the second composition comprising another cancer therapeutic agent. In a further example the first composition comprising a COX-2 inhibitor including non-steroidal anti-inflammatory drugs, beta-blockers, inhibitors of the IGFR-1 pathway, inhibitors of cathepsin, angiotensin converting enzyme inhibitors and (direct) renin inhibitors is administered after the second composition comprising another cancer therapeutic agent. In yet a further example, the first composition comprising a COX-2 inhibitor including non-steroidal anti-inflammatory drugs, beta-blockers, inhibitors of the IGFR-1 pathway, inhibitors of cathepsin, angiotensin converting enzyme inhibitors and (direct) renin inhibitors is administered before and after the second composition comprising another cancer therapeutic agent. In yet another example the second composition comprising another cancer therapeutic agent (including COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors) is administered before and after the first composition comprising a COX-2 inhibitor including non-steroidal anti-inflammatory drugs, beta-blockers, inhibitors of the IGFR-1 pathway, inhibitors of cathepsin, angiotensin converting enzyme inhibitors and (direct) renin inhibitors. In yet another example the first composition comprising a COX-2 inhibitor including non-steroidal anti-inflammatory drugs, beta-blockers, inhibitors of the IGFR-1 pathway, inhibitors of cathepsin, angiotensin converting enzyme inhibitors and (direct) renin inhibitors is administered about the same time as the second composition comprising another cancer therapeutic agent.

The delivery of a formulation comprising a COX-2 inhibitor including non-steroidal anti-inflammatory drugs, beta-blockers, inhibitors of the IGFR-1 pathway, inhibitors of cathepsin, angiotensin converting enzyme inhibitors and (direct) renin inhibitors, alone or together with another cancer therapeutic agent, including COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, over a period of time, in some instances for about 1-2 hours, about 2-4 hours, about 4-6 hours, about 6-8, or about 24 hours or longer, may also be accomplished using slow release or depot formulations, for example, as well as transdermal formulations and devices.

Strategies to improve the oral bioavailability of proteins have ranged from changing their physicochemical properties by modification of their lipophilicity and enzyme susceptibility, to adding novel functionality using transport-carrier molecules that are recognized by endogenous transport-carrier systems in the gastrointestinal tract and/or to their inclusion in specially adapted drug carrier systems. Marketed polymeric-based systems have attracted considerable attention in the controlled release in targeting particular organs/tissues, and in their ability to deliver proteins and peptides. They can effectively deliver the proteins to a target site and thus increase the therapeutic benefit, while minimizing side effects. Protein association with polymer-based carriers, such as polymeric microparticles, nanoparticles, hydrogels or patches is a useful approach to improve oral protein bioavailability. Polymer-based carriers can protect proteins from the gastrointestinal environment and allow the modulation of physicochemical and protein release properties and consequently the biological behavior. Also, from the perspective of improving oral absorption, the major effect of carriers is to increase epithelial membrane permeability, thereby leading to higher bioavailability.

Dosage forms of the compounds and formulations of the invention, extended therapeutic agent action may be achieved by affecting the rate at which the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, is released from the dosage form and/or by slowing the transit time of the dosage form through the gastrointestinal tract (see Bogner, R. H., US Pharmacist 22 (Suppl.):3-12 (1997)). The rate of drug release from solid dosage forms may be modified by the technologies described below which, in general, are based on the following: 1) modifying drug dissolution by controlling access of biologic fluids to the drug through the use of barrier coatings; 2) controlling drug diffusion rates from dosage forms; and 3) chemically reacting or interacting between the drug substance or its pharmaceutical barrier and site-specific biological fluids. Systems by which these objectives are achieved are also provided herein. In one approach, employing digestion as the release mechanism, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof are either coated or entrapped in a substance that is slowly digested or dispersed into the intestinal tract. The rate of availability of the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, is a function of the rate of digestion of the dispersible material. Therefore, the release rate, and thus the effectiveness of the therapeutic agent varies from subject to subject depending upon the ability of the subject to digest the material.

A further form of slow release dosage form of the compounds and formulations of the invention is any suitable osmotic system where semi-permeable membranes of for example cellulose acetate, cellulose acetate butyrate, cellulose acetate propionate, is used to control the release of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof. These can be coated with aqueous dispersions of enteric lacquers without changing release rate. An example of such an osmotic system is an osmotic pump device, such as the Oros™ device developed by Alza Inc. (U.S.A.).

Other devices useful in the methods of the invention utilize monolithic matrices including, for example, slowly eroding or hydrophilic polymer matrices, in which one or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are compressed or embedded.

Monolithic matrix devices comprising compounds and formulations useful in the invention include those formed using, for example, COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, dispersed in a soluble matrix, which become increasingly available as the matrix dissolves or swells; examples include hydrophilic colloid matrices, such as hydroxypropylcellulose (BP) or hydroxypropyl cellulose (USP); hydroxypropyl methylcellulose (HPMC; BP, USP); methylcellulose (MC; BP, USP); calcium carboxymethylcellulose (Calcium CMC; BP, USP); acrylic acid polymer or carboxy polymethylene (Carbopol) or Carbomer (BP, USP); or linear glycuronan polymers such as alginic acid (BP, USP), for example those formulated into microparticles from alginic acid (alginate)-gelatin hydrocolloid coacervate systems, or those in which liposomes have been encapsulated by coatings of alginic acid with poly-L-lysine membranes. Release of the therapeutic agent(s) occurs as the polymer swells, forming a matrix layer that controls the diffusion of aqueous fluid into the core and thus the rate of diffusion of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, from the system.

In such systems, the rate of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, release depends upon the tortuous nature of the channels within the gel, and the viscosity of the entrapped fluid, such that different release kinetics can be achieved, for example, zero-order, or first-order combined with pulsatile release. Where such gels are not cross-linked, there is a weaker, non-permanent association between the polymer chains, which relies on secondary bonding. With such devices, high loading of the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, is achievable, and effective blending is frequent. Devices may contain 20-80% of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, (w/w), along with gel modifiers that can enhance therapeutic agent diffusion; examples of such modifiers include sugars that can enhance the rate of hydration, ions that can influence the content of cross-links, and pH buffers that affect the level of polymer ionization. Hydrophilic matrix devices may also contain one or more pH buffers, surfactants, counter-ions, lubricants such as magnesium stearate (BP, USP) and a glidant such as colloidal silicon dioxide (USP; colloidal anhydrous silica, BP) in addition to COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, and hydrophilic matrix.

Monolithic matrix devices comprising compounds and formulations useful in the invention also include those formed using, for example, COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are dissolved in an insoluble matrix, from which the therapeutic agent(s) becomes available as the solvent enters the matrix, often through channels, and dissolves the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, particles. Examples include systems formed with a lipid matrix, or insoluble polymer matrix, including preparations formed from Carnauba wax (BP; USP); medium-chain triglyceride such as fractionated coconut oil (BP) or triglycerida saturata media (PhEur); or cellulose ethyl ether or ethylcellulose (BP, USP). Lipid matrices are simple and easy to manufacture, and incorporate the following blend of powdered components: lipids (20-40% hydrophobic solids w/w) which remain intact during the release process; e.g., channeling agent, such as sodium chloride or sugars, which leaches from the formulation, forming aqueous micro-channels (capillaries) through which solvent enters, and through which COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are released. In this system, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are embedded in an inert insoluble polymer and are released by leaching of aqueous fluid, which diffuses into the core of the device through capillaries formed between particles, and from which the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, diffuse out of the device. The rate of release is controlled by the degree of compression, particle size, and the nature and relative content (w/w) of excipients. An example of such a device is that of Ferrous Gradumet (Martindale 33rd Ed., 1360.3). A further example of a suitable insoluble matrix is an inert plastic matrix. By this method, COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are granulated with an inert plastic material such as polyethylene, polyvinyl acetate, or polymethacrylate, and the granulated mixture is then compressed into tablets. Once ingested, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are slowly released from the inert plastic matrix by diffusion. See, for example, Bodmeier, R. & Paeratakul, O., J Pharm Sci 79:32-26 (1990); Laghoueg, N., et al., Int J Pharm 50:133-139 (1989); Buckton, G., et al., Int J Pharm 74:153-158 (1991). The compression of the tablet creates the matrix or plastic form that retains its shape during the leaching of the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, and through its passage through the gastrointestinal tract. An immediate-release portion of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, may be compressed onto the surface of the tablet. The inert tablet matrix, expended of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, is excreted with the feces. An example of a successful dosage form of this type is Gradumet (Abbott; see, for example, Ferro-Gradumet, Martindale 33rd Ed., p. 1860.4).

Further examples of monolithic matrix devices useful in the methods of the invention include compositions and formulations of the invention incorporated in pendent attachments to a polymer matrix. See, for example, Scholsky, K. M. and Fitch, R. M., J Controlled Release 3:87-108 (1986). In these devices, COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, may be attached by means of an ester linkage to poly(acrylate) ester latex particles prepared by aqueous emulsion polymerization. Still further examples of monolithic matrix devices of the invention incorporate dosage forms in which the therapeutic agent(s) is bound to a biocompatible polymer by a labile chemical bond, e.g., polyanhydrides prepared from a substituted anhydride (itself prepared by reacting an acid chloride with the drug: methacryloyl chloride and the sodium salt of methoxy benzoic acid) have been used to form a matrix with a second polymer (Eudragit RL) which releases drug on hydrolysis in gastric fluid. See Chafi, N., et al., Int J Pharm 67:265-274 (1992).

Modified release forms of one or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, may also be prepared by microencapsulation. Microencapsulation is a process by which solids, liquids, or even gasses may be encapsulated into microscopic size particles through the formation of thin coatings of “wall” material around the substance being encapsulated such as disclosed in U.S. Pat. Nos. 3,488,418; 3,391,416 and 3,155,590. Gelatin (BP, USP) is commonly employed as a wall-forming material in microencapsulated preparations, but synthetic polymers such as polyvinyl alcohol (USP), ethylcellulose (BP, USP), polyvinyl chloride, and other materials may also be used. See, for example, Zentner, G. M., et al., J Controlled Release 2:217-229 (1985); Fites, A. L., et al., J Pharm Sci 59:610-613 (1970); Samuelov, Y., et al., J Pharm Sci 68:325-329 (1979). Different rates of theraeutic agent release may be obtained by changing the core-to-wall ratio, the polymer used for the coating, or the method of microencapsulation. See, for example: Yazici, E., Oner, et al., Pharmaceut Dev Technol; 1:175-183 (1996).

Other useful approaches include those in which the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are incorporated into polymeric colloidal particles or microencapsulates (microparticles, microspheres or nanoparticles) in the form or reservoir and matrix devices. See: Douglas, S. J., et al., C.R.C. Crit Rev Therap Drug Carrier Syst 3:233-261 (1987); Oppenheim, R. C., Int J Pharm 8:217-234 (1981); Higuchi, T., J Pharm Sci 52:1145-1149 (1963).

Formulations of drugs suitable for transdermal delivery are known to those skilled in the art, and are described in references such as Ansel et al., (supra). Methods known to enhance the delivery of drugs by the percutaneous route include chemical skin penetration enhancers, which increase skin permeability by reversibly damaging or otherwise altering the physicochemical nature of the stratum corneum to decrease its resistance to drug diffusion. See Shah, V., Peck, C. C., and Williams, R. L., Skin penetration enhancement: clinical pharmacological and regulatory considerations, In: Walters, K. A. and Hadgraft, J. (Eds.) Pharmaceutical skin penetration enhancement. New York: Dekker, (1993). Skin penetration enhancers suitable for formulation with COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, in transdermal drug delivery systems may be chosen from the following list: acetone, laurocapram, dimethylacetamide, dimethylformamide, dimethylsulphoxide, ethanol, oleic acid, polyethylene glycol, propylene glycol and sodium lauryl sulphate. Further skin penetration enhancers may be found in publications known to those skilled in the art. See, for example, Osborne, D. W., & Henke, J. J., Pharm Tech 21:50-66 (1997); Rolf, D., “Pharm Tech 12:130-139 (1988). In addition to chemical means, there are physical methods that enhance transdermal drug delivery and penetration of the compounds and formulations of the invention. These include iontophoresis and sonophoresis. Formulations suitable for administration by iontophoresis or sonophoresis may be in the form of gels, creams, or lotions.

Transdermal delivery, methods or formulations of the invention, may utilize, among others, monolithic delivery systems, drug-impregnated adhesive delivery systems (e.g., the Latitude™ drug-in-adhesive system from 3M), active transport devices and membrane-controlled systems. Transdermal delivery dosage forms of the invention include those which substitute the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, for the diclofenic or other pharmaceutically acceptable salt thereof referred to in the transdermal delivery systems disclosed in, by way of example, U.S. Pat. Nos. 6,193,996, and 6,262,121.

Other dosage forms include variants of the oral dosage forms adapted for suppository or other parenteral use. When rectally administered in the form of suppositories, for example, these compositions may be prepared by mixing one or more compounds and formulations of the invention with a suitable non-irritating excipient, such as cocoa butter, synthetic glyceride esters or polyethylene glycols, which are solid at ordinary temperatures, but liquify and/or dissolve in the rectal cavity to release the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof. Suppositories are generally solid dosage forms intended for insertion into body orifices including rectal, vaginal and occasionally urethrally and can be long acting or slow release. Suppositories include a base that can include, but is not limited to, materials such as alginic acid, which will prolong the release of the pharmaceutically acceptable active ingredient over several hours (5-7).

Transmucosal administration of the compounds and formulations useful in the invention may utilize any mucosal membrane but commonly utilizes the nasal, buccal, vaginal and rectal tissues. Formulations suitable for nasal administration of the compounds and formulations of the invention may be administered in a liquid form, for example, nasal spray, nasal drops, or by aerosol administration by nebulizer, including aqueous or oily solutions of the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof. Formulations for nasal administration, wherein the carrier is a solid, include a coarse powder having a particle size, for example, of less than about 100 microns, preferably less, most preferably one or two times per day than about 50 microns, which is administered in the manner in which snuff is taken, i.e., by rapid inhalation through the nasal passage from a container of the powder held close up to the nose. Compositions in solution may be nebulized by the use of inert gases and such nebulized solutions may be breathed directly from the nebulizing device or the nebulizing device may be attached to a facemask, tent or intermittent and COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof may be administered orally or nasally from devices that deliver the formulation in an appropriate manner. Formulations may be prepared as aqueous solutions for example in saline, solutions employing benzyl alcohol or other suitable preservatives, absorption promoters to enhance bio-availability, fluorocarbons, and/or other solubilising or dispersing agents known in the art.

Compositions may be prepared according to conventional methods by dissolving or suspending an amount of a COX-2 inhibitor including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, in a diluent. The amount of therapeutic agent is from between 0.1 mg to 1000 mg per ml of diluent. In some examples, dosage forms of 100 mg and 200 mg of therapeutic agent(s) are provided. By way of example only, the amount of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, may range from about 1 mg to about 750 mg or more (for example, about 1 mg, about 10 mg, about 25 mg, about 50 mg, about 100 mg, about 150 mg, about 200 mg, about 250 mg, about 400 mg, about 500 mg, about 600 mg, about 750 mg, about 800 mg, about 1000 mg, and about 1200 mg). Other amounts within these ranges may also be used and are specifically contemplated though each number in between is not expressly set out.

Therapeutic agents, including COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, can be provided and administered in forms suitable for once-a-day dosing. An acetate, phosphate, citrate or glutamate buffer may be added allowing a pH of the final composition to be from about 5.0 to about 9.5; optionally a carbohydrate or polyhydric alcohol tonicifier and, a preservative selected from the group consisting of m-cresol, benzyl alcohol, methyl, ethyl, propyl and butyl parabens and phenol may also be added. Water for injection, tonicifying agents such as sodium chloride, as well as other excipients, may also be present, if desired. For parenteral administration, formulations are isotonic or substantially isotonic to avoid irritation and pain at the site of administration.

The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydroxyl ion added does not exceed the capacity of the buffer system to neutralize it.

Maintaining the pH of the formulation in the range of approximately 5.0 to about 9.5 can enhance the stability of the parenteral formulation of the present invention. Other pH ranges, for example, include, about 5.5 to about 9.0, or about 6.0 to about 8.5, or about 6.5 to about 8.0, or, preferably, about 7.0 to about 7.5.

The buffer used may be selected from any of the following, for example, an acetate buffer, a phosphate buffer or glutamate buffer, the most preferred buffer being a phosphate buffer. Carriers or excipients can also be used to facilitate administration of the compositions and formulations of the invention. Examples of carriers and excipients include calcium carbonate, calcium phosphate, various sugars such as lactose, glucose, or sucrose, or types of starch, cellulose derivatives, gelatin, polyethylene glycols and physiologically compatible solvents. A stabilizer may be included, but will generally not be needed. If included, however, an example of a stabilizer useful in the practice of the invention is a carbohydrate or a polyhydric alcohol. The polyhydric alcohols include such compounds as sorbitol, mannitol, glycerol, xylitol, and polypropylene/ethylene glycol copolymer, as well as various polyethylene glycols (PEG) of molecular weight 200, 400, 1450, 3350, 4000, 6000, and 8000). The carbohydrates include, for example, mannose, ribose, trehalose, maltose, inositol, lactose, galactose, arabinose, or lactose.

Isotonicity agents, or agents to maintain isotonicity, may also be used or included.

The United States Pharmacopeia (USP) states that anti-microbial agents in bacteriostatic or fungistatic concentrations must be added to preparations contained in multiple dose containers. They must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents should be evaluated to ensure compatibility with all other components of the formula, and their activity should be evaluated in the total formula to ensure that a particular agent that is effective in one formulation is not ineffective in another. It is not uncommon to find that a particular agent will be effective in one formulation but not effective in another formulation. While the preservative for use in the practice of the invention can range from 0.005 to 1.0% (w/v), the preferred range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid. A detailed description of each preservative is set forth in “Remington's Pharmaceutical Sciences” as well as Pharmaceutical Dosage Forms: Parenteral Medications, Vol. 1, 1992, Avis et al. For these purposes, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof may be administered parenterally (including subcutaneous injections, intravenous, intramuscular, intradermal injection or infusion techniques) or by inhalation spray in dosage unit formulations containing conventional non-toxic pharmaceutically acceptable carriers, adjuvants and vehicles.

If desired, the parenteral formulation may be thickened with a thickening agent such as a methylcellulose. The formulation may be prepared in an emulsified form, either water in oil or oil in water. Any of a wide variety of pharmaceutically acceptable emulsifying agents may be employed including, for example, acacia powder, a non-ionic surfactant or an ionic surfactant. It may also be desirable to add suitable dispersing or suspending agents to the pharmaceutical formulation. These may include, for example, aqueous suspensions such as synthetic and natural gums, e.g., tragacanth, acacia, alginate, dextran, sodium carboxymethylcellulose, methylcellulose, polyvinyl-pyrrolidone or gelatin.

It is possible that other ingredients may be present in a parenteral pharmaceutical formulation useful the invention. Such additional ingredients may include wetting agents, oils (e.g., a vegetable oil such as sesame, peanut or olive), analgesic agents, emulsifiers, antioxidants, bulking agents, tonicity modifiers, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention. Regarding pharmaceutical formulations, see also, Pharmaceutical Dosage Forms: Parenteral Medications, Vol. 1, 2nd ed., Avis et al., Eds., Mercel Dekker, New York, N.Y. 1992.

Suitable routes of parenteral administration include intramuscular, intravenous, subcutaneous, intraperitoneal, subdermal, intradermal, intraarticular, intrathecal and the like. Mucosal delivery is also permissible. The dose and dosage regimen will depend upon the weight and health of the subject.

In addition to the above means of achieving extended drug action, the rate and duration of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, delivery may be controlled by, for example by using mechanically controlled drug infusion pumps.

The COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, can be administered in the form of a depot injection that may be formulated in such a manner as to permit a sustained release of the therapeutic agents. The therapeutic agents can be compressed into pellets or small cylinders and implanted subcutaneously or intramuscularly. The pellets or cylinders may additionally be coated with a suitable biodegradable polymer chosen so as to provide a desired release profile. The COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof may alternatively be micropelleted. The micropellets using bioacceptable polymers can be designed to allow release rates to be manipulated to provide a desired release profile. Alternatively, injectable depot forms can be made by forming microencapsulated matrices of the therapeutic agents in biodegradable polymers such as polylactide-polyglycolide. Depending on the ratio of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, to polymer, and the nature of the particular polymer employed, the rate of therapeutic agent release can be controlled. Depot injectable formulations can also be prepared by entrapping the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof in liposomes, examples of which include unilamellar vesicles, large unilamellar vesicles and multilamellar vesicles. Liposomes can be formed from a variety of phospholipids, such as cholesterol, stearyl amine or phosphatidylcholines. Depot injectable formulations can also be prepared by entrapping the therapeutic agent in microemulsions that are compatible with body tissue. By way of example, reference is made to U.S. Pat. Nos. 6,410,041 and 6,362,190.

Implantable infusion devices may employ inert material such as biodegradable polymers listed above or synthetic silicones, for example, cylastic, silicone rubber or other polymers manufactured by the Dow-Corning Corporation. The polymer may be loaded with COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof and any excipients. Implantable infusion devices may also comprise a coating of, or a portion of, a medical device wherein the coating comprises the polymer loaded with COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, and any excipient. Such an implantable infusion device may be prepared as disclosed in U.S. Pat. No. 6,309,380 by coating the device with an in vivo biocompatible and biodegradable or bioabsorbable or bioerodible liquid or gel solution containing a polymer with the solution comprising a desired dosage amount of therapeutic agent and any excipients. The solution is converted to a film adhering to the medical device thereby forming the implantable therapeutic-deliverable medical device. An implantable infusion device may also be prepared by the in situ formation of a therapeutic agent containing solid matrix as disclosed in U.S. Pat. No. 6,120,789. Implantable infusion devices may be passive or active, as known in the art.

Also useful in methods of the invention are microemulsions, i.e., such as fluid and stable homogeneous solutions composed of a hydrophilic phase, a lipophilic phase, at least one surfactant (SA) and at least one cosurfactant (CoSA). Examples of suitable surfactants include mono-, di- and triglycerides and polyethylene glycol (PEG) mono- and diesters. A cosurfactant, also sometimes known as “co-surface-active agentm,” is a chemical compound having hydrophobic character, intended to cause the mutual solubilization of the aqueous and oily phases in a microemulsion. Examples of suitable co-surfactants include ethyl diglycol, lauric esters of propylene glycol, oleic esters of polyglycerol, and related compounds.

Therapeutic agents, including COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, may also be delivered using various polymers to enhance bioavailability by increasing adhesion to mucosal surfaces, by decreasing the rate of degradation by hydrolysis or enzymatic degradation of the therapeutic agents, and by increasing the surface area of the therapeutic agent relative to the size of the particle. Suitable polymers can be natural or synthetic, and can be biodegradable or non-biodegradable. Delivery of low molecular weight active agents may occur by either diffusion or degradation of the polymeric system. Representative natural polymers include proteins such as zein, modified zein, casein, gelatin, gluten, serum albumin, and collagen, polysaccharides such as cellulose, dextrans, and polyhyaluronic acid. Synthetic polymers are generally preferred due to the better characterization of degradation and release profiles. Representative synthetic polymers include polyphosphazenes, poly(vinyl alcohols), polyamides, polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate). Synthetically modified natural polymers include cellulose derivatives such as alkyl celluloses, hydroxyalkyl celluloses, cellulose ethers, cellulose esters, and nitrocelluloses. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. Each of the polymers described above can be obtained from commercial sources such as Sigma Chemical Co., St. Louis, Mo., Polysciences, Warrenton, Pa., Aldrich Chemical Co., Milwaukee, Wis., Fluka, Ronkonkoma, N.Y., and BioRad, Richmond, Calif. or can be synthesized from monomers obtained from these suppliers using standard techniques.

The polymers described above can be separately characterized as biodegradable, non-biodegradable, and bioadhesive polymers. Representative synthetic degradable polymers include polyhydroxy acids such as polylactides, polyglycolides and copolymers thereof, poly(ethylene terephthalate), poly(butic acid), poly(valeric acid), poly(lactide-co-caprolactone), polyanhydrides, polyorthoesters and blends and copolymers thereof. Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers. Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof. Hydrophilic polymers and hydrogels tend to have bioadhesive properties. Hydrophilic polymers that contain carboxylic groups (e.g., poly[acrylic acid]) tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, while others are hydrogels. Polymers such as hydroxypropylmethylcellulose acetate succinate (HPMCAS), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP) may be utilized to enhance the bioavailability of therapeutic agents with which they are complexed. Rapidly bioerodible polymers such as poly(lactide-co-glycolide), polyanhydrides, and polyorthoesters, whose carboxylic groups are exposed on the external surface as their smooth surface erodes, can also be used for bioadhesive therapeutic agent systems. In addition, polymers containing labile bonds, such as polyanhydrides and polyesters, are well known for their hydrolytic reactivity. Their hydrolytic degradation rates can generally be altered by simple changes in the polymer backbone. Upon degradation, these materials also expose carboxylic groups on their external surface, and can also be used as B natriuretic signal peptide fragment agent delivery systems.

Other agents that may enhance bioavailability or absorption of one or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof can act by facilitating or inhibiting transport across the intestinal mucosa. For example, agents that increase blood flow, such as vasodilators, may increase the rate of absorption of orally administered therapeutic agents by increasing the blood flow to the gastrointestinal tract. Vasodilators constitute another class of agents that may enhance the bioavailability of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof.

Other mechanisms of enhancing bioavailability of the compositions and formulations useful in the invention include the inhibition of reverse active transport mechanisms. For example, it is now thought that one of the active transport mechanisms present in the intestinal epithelial cells is p-glycoprotein transport mechanism which facilitates the reverse transport of substances, which have diffused or have been transported inside the epithelial cell, back into the lumen of the intestine. Inhibition of this p-glycoprotein mediated active transport system will cause less drug to be transported back into the lumen and will thus increase the net drug transport across the gut epithelium and will increase the amount of drug ultimately available in the blood. Various p-glycoprotein inhibitors are well known and appreciated in the art. These include, water soluble vitamin E; polyethylene glycol; poloxamers including Pluronic F-68; Polyethylene oxide; polyoxyethylene castor oil derivatives including Cremophor EL and Cremophor RH 40; Chrysin, (+)-Taxifolin; Naringenin; Diosmin; Quercetin; and the like.

Thus, while the delivery period will be dependent upon both the condition and the agent and the therapeutic effect which is desired, continuous or slow-release delivery for about 0.5-1 hour, about 1-2 hours, about 2-4 hours, about 4-6 hours, about 6-8, or about 24 hours or longer is provided. In accordance with the present invention, this is achieved by inclusion of a COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, optionally alone or together with another cancer therapeutic agent, in a formulation together with a pharmaceutically acceptable carrier or vehicle, particularly in the form of a formulation for continuous or slow-release administration.

The routes of administration and dosages described herein are intended only as a guide since a skilled physician will consider the optimum route of administration and dosage for any particular patient and condition.

Any of the methods of treating a subject having or at risk for cancer may utilize the administration of any of the doses, dosage forms, formulations, and/or compositions herein described.

### Pharmaceutical Compositions

The present invention is directed to pharmaceutical compositions and their methods of use for treating or managing cancer wherein the composition comprises a therapeutically effective amount of COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, alone or together with another cancer therapeutic agent.

Accordingly, in one aspect, the invention provides compositions for use in treating or managing cancer, which comprises or consists essentially of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, alone or together with another cancer therapeutic agent. In a preferred example, the composition further comprises a pharmaceutically acceptable carrier or vehicle.

### Kits, Medicaments and Articles of Manufacture

The drug combinations, compositions and formulations described herein may also be used in the manufacture of the medicament for treating or managing cancer.

In one aspect, the invention provides a kit for treating or managing cancer comprising one or more combinations, compositions or formulations described herein. For example, the invention includes a kit comprising a combination, composition or formulation comprising a therapeutically effective amount of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, alone or in combination with one or more cancer therapeutic agents. For example, the kit may include a composition comprising an effective amount of a COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof and or more of the following: nitrates, β-blockers, calcium channel blockers (particularly for stable or unstable angina, but also for heart failure in the case of p-blockers); diuretic agents, vasodilator agents, positive inotropes, ACE inhibitors and aldosterone antagonists, e.g. spironolactone (particularly for heart failure); blood thinning therapeutics (e.g., aspirin, heparins, warfarins) and nitroglycerin (particularly for MI). Kits may also include combinations, compositions and formulations comprising or consisting essentially of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, alone or in combination with (e.g., in physical combination, provided as a combined preparation) one or more anti-cancer therapies.

Articles of manufacture are also provided comprising a vessel containing a combination, composition or formulation of the invention (in any dose or dose form or device) as described herein and instructions for use for the treatment of a subject. For example, in another aspect, the invention includes an article of manufacture comprising a vessel containing a therapeutically effective amount of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, alone or in combination with one or more other cancer therapeutic agents.

### Treatment

The combinations, compositions and formulations of the present invention may be used for preventing and/or treating cancer in a patient in need thereof.

The inventions also include methods of treatment of a subject having cancer or at risk for recurrence of cancer, comprising administering to the subject a therapeutically effective amount of a combination, composition and/or formulation described herein. In one non-limiting example, the cancer is selected from squamous cell carcinoma of the upper aerodigestive tract (including oral cavity), squamous cell carcinoma of the skin, melanoma, lung cancer, breast cancer, kidney cancer, brain cancer, bowel cancer, thyroid cancer, prostate cancer, lymphoma, leukemia and sarcomas.

The inventions include methods of treating a subject having cancer or at risk for recurrence of cancer, comprising administering a therapeutically effective amount of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, and a pharmaceutically acceptable carrier. In one example, the non-steriodal antiinflammatory drug includes, but is not limited to, Salicylates, including, but not limited to, Salicyclic Acid, Acetylsalicylic Acid, Salsalate, Diflunisal; Propionic Acid derivatives, including, but not limited to, Ibuprofen, Dexibuprofen, Naproxen, Denoprofen, Ketoprofen, Dexketoprofen, Flubirpofen, Oxaprozin and loxoprofen; Acetic Acid derivatives, including, but not limited to, Indomethacin, Tolmetin, Sulindac, Etodolac, Ketorolac, Diclofenac, Aceclofenac, Nabumetone; Enolic Acid (Oxicam) derivatives, including, but not limited to, Piroxicam, Meloxicam, Tenoxicam, Droxicam, lornoxicam, Isoxicam and Phenylbutazone; Anthranilic Acid derivatives, including, but not limited to, Mefenamic Acid, Meclofenamic Acid, Flufenamic Acid, Tolfenamic Acid; COX-2 Inhibitors, including, but not limited to, Celecoxib, Rofecoxib, Valdecoxib, Parecoxib, lumiracoxib, Etoricoxib; Sulfonamides, including, but not limited to, Nimesulide; Clonixin; and Licofelone. In other examples, the beta-blocker includes, but is not limited to, Acebutolol (Sectral), Atenolol (Tenormin), Betaxolol (Betoptic), Bisoprolol (Cardicor, Emcor, Zebeta), Carteolol (Teoptic), Carvedilol (Coreg, Eucardic), Celiprolol (Celectol), Labetalol (Trandate), Levobunolol (Betagan), Metipranolol (Metipranolol Minims), Metoprolol (Betaloc, Lopresor, Lopressor, Toprol XL), Nadolol (Corgard), Nebivolol (Bystolic, Nebilet), Oxprenolol (Trasicor), Pindolol (Visken), Propranolol (Inderal LA), Sotalol (Beta-Cardone, Sotacor), and Timolol (Betim, Nyogel, Timoptol). In yet a further example, the cathepsin inhibitor includes, but is not limited to, Curcumin, Cystatin B, Cystatin C, Cysteine peptidase inhibitor E64, [Pt(dmba)(aza-N1)(dmso)] complex 1 (a potential anti-tumoral drug with lower IC50 than cisplatin in several tumoral cell lines), 2,3,7,8-Tetrachlorodibenzo-p-dioxin (TCDD), CA-074Me, Lipidated CtsB inhibitor incorporated into the envelope of a liposomal nanocarrier (LNC-NS-629), Proanthocyanidin (PA) and ahpatinin Ac (1) and ahpatinin Pr (2). In yet another example, the angiotensin converting enzyme inhibitor includes, but is not limited to, Benazepril (Lotesin), Captopril (Capoten), Cilazipril, Enalapril (Vasotec, Renitec), Fosinopril (Monopril), Lisinopril (Lisodur, Lopril, Novatec, Prinivil, Zestril), Moexipril, Perindopril (Coversay, Aceon), Quinapril (Accupril), Ramipril (Altace, Tritace, Ramace, Ramiwin), Trandolapril, Delapril, Zofenopril and Imidapril. In yet another example, the IGFR-1 pathway inhibitor includes but is not limited to, metformin, tyrphostins such as AG538 and AG1024, pyrrolo(2,3-d)-pyrimidine derivatives such as NVP-AEW541 and Figitumumab (also called CP-751871). In yet another example, the renin inhibitor includes but is not limited to, Aliskiren.

In other examples, the two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are administered in a single dose. In another example, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are administered in more than one dose. In yet another example, the COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, are administered continuously over a period of time, for example a predetermined period of time.

In another aspect, the inventions include methods for treatment of a patient, comprising administering to the patient a therapeutically effective amount of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors, including combinations thereof, wherein the administration is after the onset of one or more symptoms of cancer.

The inventions also include methods for treating a patient suffering from squamous cell carcinoma of the upper aerodigestive tract (including oral cavity), comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from squamous cell carcinoma of the skin, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from melanoma, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from lung cancer, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from breast cancer, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from kidney cancer, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from brain cancer, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from bowel cancer, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from prostate cancer, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from lymphoma, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from leukemia, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from sarcomas, comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from oral cavity squamous cell carcinoma (OCSCC), comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from recurrent locally advanced and/or metastatic head and neck cutaneous squamous cell carcinoma (HNcSCC), comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from recurrent malignant melanoma (MM), comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

The inventions also include methods for treating a patient suffering from recurrent glioblastoma multiforme (GBM), comprising administration of two or more COX-2 inhibitors including non-steroidal anti-inflammatory drugs, beta-blockers, inhibitors of the IGFR-1 pathway, inhibitors of cathepsin, angiotensin converting enzyme inhibitors and (direct) renin inhibitors. In a further example, the administration is continuous over a period of time, including a predetermined period of time.

In another aspect, the treated subject is a mammal, preferably a human. Other rmammals include domestic and farm animals, and zoo, sports, or pet animals, such as dogs, horses, and cats.

Any of the methods of treating a subject having or suspected of having or predisposed to a disease, disorder, and/or condition referenced or described herein may utilize the administration of any of the doses, dosage forms, formulations, combinations, compositions and/or devices herein described.

Any reference to prior art documents in this specification is not to be considered an admission that such prior art is widely known or forms part of the common general knowledge in the field.

The invention is further described with reference to the following examples. It will be appreciated that the invention as claimed is not intended to be limited in any way by these examples.

### EXAMPLE 1: TREATMENT OF PATIENT WITH STAGE IV ADENOCARCINOMA OF LUNG

### “Patient X” Data

66-year old male
Past medical history
Left total hip replacement May 2011
Nasal fracture September 2006
Life-long non-smoker

### Patient X—Overview

Patient X was diagnosed with a very aggressive and advanced stage adenocarcinoma of the lung (Stage IV), with extensive and widespread bony and soft tissue metastases in October 2010. Patient X was given the option of palliative radiotherapy.

Patient X went into remission with chemotherapy but developed early relapse and went on to Tarceva (thymidine kinase inhibitor (TKI) that targets the EGFR exon 19 mutation). This led to remission but Patient X relapsed in early April 2015 and had undergone limited palliative XRT to one of the metastasis. Between June 2015 and January 2017 he underwent ‘RAS modulation’ using the drug combinations of the present invention (further details given below). Over this time the cancer has taken an indolent course with slow progression. Patient X is still alive and fully functional.

Applicants are not aware of any reported case of stage IV adenocarcinoma of the lung where the patient has survived >20 months. Typically, there is a 50% mortality within 5 months if the adenocarcinoma is left untreated; 10-15% survival at 1 year; 4% survival at >5 years.

Typically when the disease relapses it takes a rampant course (life expectancy 3-6 months). Applicants provide the first evidence to demonstrate that RAS modulation has had a significant therapeutic benefit to the patient and has radically changed the pathology of the cancer (now 21 months since relapse).

Treatment Protocol (incl. Chronological History)

### 15 Oct. 2010

Initially Patient X presented to general practitioner (GP) with 6-week history of cough and reduction of breathing capacity and an injured wrist pushing a car. Patient X had also suffered a fall on his right hand on 3 Sep. 2010. Pain in the hand (base of thumb) and shoulder. X-Ray on 10 Oct. 2010 showed a lytic lesion on the scaphoid. CXR showed a 3 cm lesion left lung base.

### 20 Oct. 2010

CT Scan showed a 1.5 cm air-space consolidation left mid zone at the site of a 3 cm lesion shown on previous CXR. Ten 4 mm lesions in the periphery of right middle and lower lobes. Extensive nodal disease in the chest, and axillae.

### 22 Oct. 2010

Left lower lobe lesion biopsied—insufficient tissue to make a diagnosis.

### 25 Oct. 2010

PET CT showed extensive widespread bony (spine, scapula, clavicle, humerus, pelvis) and soft tissue (including lung) and lymph node (including axillary) metastases, from a small primary.

### 27 Oct. 2010

Patient X underwent mediastinoscopy and biopsy which confirmed adenocarcinoma with EGFR exon 19 mutation.
Diagnosis: Stage IV lung poorly differentiated adenocarcinoma left lung
Stats: 50% mortality within 5 months if untreated
   10-15% survival at 1 year

### 9 Nov. 2010

Patient X underwent 6 cycles of chemotherapy: Carboplatin/Pemetrexed and Bevacizumab (Avastin) and completed on 15 Mar. 2011.
Patient X has been on life-style diet/exercise change since diagnosis including regular intake of turmeric.

### 22 Mar. 2011

Repeat PET CT showed a complete metabolic response.

### 31 May 2011

Repeat PET CT showed early relapse with increased avidity in bones and small upper lobe nodules and avid small nodes below the diaphragm and left lower lobe, new tiny lesions right upper lobe, of the lungs.

### 2 Jun. 2011

Patient X commenced Tarceva (150 mg once daily).
Repeat PET CTs on 12 Jul. 2011, 11 Oct. 2011, 20 Mar. 2012, 7 Aug. 2012, 21 May 2013 and 5 Nov. 2013 showed remission of the cancer.

### 1 Apr. 2015

Repeat PET CT showed a new right para-aortic node with high avidity, behind the left renal vein, with a number of lung lesions that demonstrated slow growth with low avidity.

### 28 Apr. 2015

Patient X underwent stereotactic radiotherapy (RT) to the right para aortic node at 30 Gy.

### June-July 2015

Commenced RAS modulation:
Aspirin: 300 mg once daily

### Start Date: 20 Jun. 2015

### 28 Jul. 2015

Repeat PET CT demonstrated static appearance with low levels of activity remaining at both the primary and left lung metastasis sites.

### July-August 2015

Further RAS modulation:
Aspirin: 300 mg once daily
Aliskiren (Rasilez): 150 mg once daily.

### Start Date: 29 Jul. 215

### August 2015

Repeat PET CT showed further avidity in the para-aortic node below the original stereotactic RT-treated area and there was further discussion of the possibility of further stereotactic RT, open biopsy or lobectomy (for the lung primary). Decided to proceed with stereotactic RT which was subsequently deemed not feasible based on the results of a repeat PET CT.

### September-October 2015

Further RAS modulation:
Aspirin: 300 mg once daily
Aliskiren (Rasilez): 150 mg once daily
Propranolol: 20 mg three times daily

### Start Date: 29 Sep. 2015

### 20 Oct. 2015

### Repeat PET CT

Reported increased avidity in a new adjacent para aortic node plus partial reactivation of metabolic activity in the presumed primary and one lung metastatic lesion.

### October-November 2015

Further RAS modulation:
Aspirin: 300 mg once daily
Aliskiren (Rasilez): 150 mg once daily
Propranolol: 40 mg three times daily

### Start Date: 22 Oct. 2015

### 5 Nov. 2015

Patient X was seen by a medical oncologist. Noted that there were 4 lesions, 2 of which were known for some time and are slowly growing (suggested to continue monitor), with the third being the primary which had grown for 4 mm over 3 months and had now demonstrated low avidity. The area of most immediate concern was a new node adjacent to the left renal vein which demonstrated avidity. Discussion occurred regarding the need to treat this with stereotactic RT or by endoscopic surgery.

### 6 Nov. 2015

Patient X was seen by a radiation oncologist to discuss stereotactic RT to node adjacent to left renal artery. Decided to proceed and also to treat the active primary lung disease, following a lung biopsy.

### 17 Nov. 2015

Patient X underwent a biopsy of lung primary which showed squamous differentiation with identical EGFR mutation (exon 19) to the original primary and was considered ‘adeno-squamous carcinoma’. EGFR mutation tests showed deletion of exon 19 as an activating mutation. T790M mutation in exon 20 was not detected.

### 18 Nov. 2015

Repeat PET CT showed no new abnormality in the head, neck, thorax, abdomen, pelvis apart from the following: previously known left lung base primary and metastasis as well as right para aortic nodes all similar if not slightly increased avidities, and possibly minimally but no significant increase in size. There was a new micrometastasis noted in the chest, right of the aorta in the lower thorax, and another micro-metastasis just to the left of the abdominal para-aortic node. No evidence of re-activation of other known sites. Mild increase in avidity of level V node on the right side of the neck.

### 20 Nov. 2015

Radiation oncologist reviewed the repeat PET CT and decided against stereotactic RT.

### November-December 2015

Further enhanced RAS modulation:
Aspirin: 300 mg once daily
Aliskiren (Rasilez): 150 mg once daily
Propranolol: 60 mg three times daily

### Start Date: 20 Nov. 2015

+ Cilazapril: 2.5 mg once daily

### Start Date: 26 Nov. 2015

### 16 Dec. 2015

Repeat PET CT showed indolent course of the disease. The most recent scan showed that the lesion in the para-aortic area that had become avid in August 2015, appeared to have changed architecturally (?effacing). In addition, the 2 cm node in level V on the right side of the neck just behind the posterior border of the sternomastoid muscle that was not avid at the time of the diagnosis in October 2010, had now demonstrated slight avidity. A 1 cm node 2 cm above the level V node, in level III that was highly avid in October 2010 and became quiescent had become avid in the last ×2 scans with the recent scan showing a further increase in avidity.

### 22 Nov. 2015

Proceeded with excisional biopsy of the right neck level V node and level III nodes. At the time, small nodes in between these two nodes were removed. Histology showed metastatic poorly differentiated lung adenocarcinoma in the level III node. The cells were positive for CK1/3, CK7, p63 and focally positive for TTF-1. Positivity for p63 was considered ‘unusual’ and ‘may be a post-treatment phenomenon.’ The level V node and those taken between the level V and level II nodes showed a monotonous small lymphocyte proliferation with few residual follicles present in the cortex. The atypical lymphocytes are positive for CD20, CD79a, BCL-2, CD5 and CD23. The Ki-67 index was low. The features are strongly suggestive of a small lymphocytic lymphoma and there was no evidence of metastatic adenocarcinoma. Tests for EGFR T790 mutations, PD1, PDL1 were requested. These were eventually reported in a supplemental report on 5 Feb. 2016 showing EGFR exon 19 deletion was detected. BRAF, KRAS and NRAS mutation were not detected.

### December 2015-April 2016

Further RAS modulation:
Aspirin: 300 mg once daily
Aliskiren (Rasilez): 150 mg once daily
Propranolol: 60 mg three times daily
Cilazapril: 2.5 mg once daily
Curcumin*: 90 mg twice daily

### Start Date: 22 Dec. 2015

+ Doxycycline: 100 mg once daily

### Start Date: 20 Feb. 2016

*Curcuma activa-curcumin phospholipid complex 500 mg containing curcumin 90 mg

### 7 Apr. 2016

Repeat PET CT showed slight increase in avidities but with minimal increase in sizes of the lesions, as well as a new metastatic lesion noted in the right lung base, although considered stable disease within trial criteria.

### April-October 2016

Doxycline was stopped and Metformin added.
RAS modulation:
Aspirin: 300 mg once daily
Aliskiren (Rasilez): 150 mg once daily
Propranolol: 60 mg three times daily
Cilazapril: 2.5 mg once daily
Curcumin*: 90 mg twice daily
Metformin: 500 mg once daily
Start date 19: April 2016
Administration of the above treatment regime occurred once daily for two weeks, and twice daily thereafter.
*Curcuma activa-curcumin phospholipid complex 500 mg containing curcumin 90 mg

### EXAMPLE 2: TREATMENT OF PATIENT WITH ENDOMETRIOMA

Patient LT. 42 year-old female

### 20 Feb. 2017

Menarche aged 12. First diagnosed with endometriosis in 1988, aged 13/14, when she first experienced haemoptysis. Presented with a significant episode of haemoptysis (½ cupful of fresh blood) and coughing when she injured left knee playing hockey in March 2014. CXR then showed a 45 mm soft tissue tumour in the left lung. CT scan on 16.4.14 showed the lesion centred on the oblique fissure. Repeat CT on 15.9.14 showed the lesion to be 42×34 mm. Provisional diagnosis of endometrioma was made by a respiratory physician. Haemoptysis with sharp chest pains and right shoulder tip pains particularly related to her periods. Now has minor haemoptysis daily with chest and right shoulder tip pains.

Could not get pregnant and had had 4 miscarriages. Subsequently became had 2 successful pregnancies and deliveries.

Underwent bilateral oophorectomies (and cholecystectomy) and had been on Letrozole for 2 years which causes significant loss of bone density.

Had had a PET CT in December 2016 which showed low avidity of the tumour, consistent with an endometrioma. Had had regular CT scans to monitor the lung lesion and the CT on Jul. 11, 2016 showed the lesion measured 48×42 mm, an increase in size by 0.6 cm over 6 months.

Referred by gynaecologist to a cardiothoracic surgeon who advised that removal of the lesion will involve left pneumonectomy. Referred by cardiothoracic surgeon to us for consideration of novel treatment.

Attempted core biopsy of the lung lesion was not helpful—blood only.

### 31 May 2017

A repeat CT on 15.5.17 showed the lesion measuring 49×44 mm

CXR on 31.5.17 showed the lesion measuring 52×51×48 mm

### 1 Jun. 2017

Commenced novel treatment consisting of:
1). Aspirin 300 mg daily
2.) Aliskiren: 150 mg daily for 2 weeks and then increase to 150 mg twice daily,
3. Curcumin with BioPerine 1000 mg twice daily

### 14 Jun. 2017

No haemoptysis within a week of initiation of treatment. Chest and shoulder tip pains subsiding.

### 8 Aug. 2017

CXR of 4.8.17 showed lesion measures 51×50×47 mm (smaller than 31.5.17)

Aspirin was replaced by celecoxib 100 mg daily

Patients feels much better with no recurrence of haemoptysis.

### 5 Oct. 2017

CXR on May 10, 2017 showed the lesion measuring 50×49×38 mm

No haemoptysis.

### EXAMPLE 3: TREATMENT OF PATIENT WITH SQUAMOUS CELL CARCINOMA

Patient LH. 88 year-old female with a biopsy-proven 6.5×1.5 cm right sided squamous cell carcinoma of the mandibular alveolus/retromolar trigone with invasion of the underlying mandible and a 2 cm ipsilateral level II node. CT scan confirmed the primary and the neck metastasis and bony invasion which is also shown on OPG. No lung metastasis. TNM staging: T4N1M0. The patient was edentulous. The patient lived alone and was generally frail. Her medications included atenolol 50 mg daily, colchicine 600 mg daily, aspirin 100 mg daily and benzofluarizide 2.5 mg daily.

The patient was evaluated at the Head and Neck MDT and was offered treatment options including curative treatment (surgery and XRT) and palliative care. The patient declined activeconventional treatment and was referred to the Hospice for palliative care.

The patient was offered and began the Applicant's novel cancer treatment that targets the cancer stem cells by modulation of the renin-angiotensin system. Benzofluarizide, atenolol and aspirin were stopped and propranolol 160 mg daily and curcumin 1000 mg twice daily and celebrex 100 mg daily were commenced.

Five weeks later there was reduction of the size of the tumour with the edges effacing. The level II node had reduced in size dramatically measuring 8 mm. The dosage of propranolol was reduced to 40 mg daily because the patient was feeling ‘weak in the legs’ and had two episodes of falls. There was minimal discomfort in the mouth.

Thirteen weeks after initiation of the treatment there was an overall 40% reduction of the tumour with ongoing effacing of the edges of the tumour and epithelialisation of the upper third of the tumour. There was minimal discomfort in the mouth. The level II node was unchanged at 8 mm.

Five months after initiation of treatment. The patient had developed swelling in the right leg associated with erythema and mild swelling in the left leg. Commenced on doxycycline and frusemide by GP. The tumour may have increased in size slightly. No pain. The neck node remained unchanged.

### EXAMPLE 4: CLINICAL TRIAL

### Trial Design

This is an open-labelled ‘proof of concept’ interventional study. The patients being recruited for this study have exhausted treatment options and are generally expected to have limited life expectancy with a deteriorating quality of life. For these patients the average survival and their quality of life is relatively short with a median survival times of 7-10 months for recurrent head and neck SCC³⁷, 6-8 months for metastatic melanoma³⁸, and 12-15 months for GBM³⁹ from diagnosis with a much shorter survival for recurrent GBM. Each subject will serve as his/her own control. The proposed study will record and compare ‘before’ (baseline) and ‘after’ data including the quality of life and the length of survival of the patients who have been treated.

### Inclusion Criteria

1. Patients with the types of cancer listed under (2) below who have exhausted conventional treatment option(s), where further conventional treatment has a low prospect of a beneficial outcome. They will have a good performance status with a Karnofsky score⁴⁰ of at least 60. The patients may be undergoing palliative care.
2. Types of recurrent advanced cancers to be included in the study (25 patients for each group) are:
   a. oral cavity squamous cell carcinoma (OCSCC)
   b. locally advanced and/or metastatic head and neck skin squamous cell carcinoma (HNsSCC)
   c. glioblastoma multiforme (GBM)
   d. malignant melanoma (MM)
3. The patients will be referred by their specialists or general practitioners or by word of mouth

### Exclusion Criteria

1. Cancer patients who have a life expectancy of less than 6 months
2. Patients with a Karnovsky score <60.
3. Patients who are not able to swallow medication (tablets and capsules)
4. Patients who are on medications that increase renin levels, such as calcium channel blockers and diuretics
5. Patients who are not motivated including non-compliance, e.g., continue to smoke, abuse alcohol
6. Children less than 16 years
7. Patients older than 80 years
8. Patients who are not competent to give consent.
9. Patients who are on other studies or trials
10. Presence of contraindications to any of the study treatments including asthma/CORD, blood pressure (BP) 5100 mmHg systolic, drug allergies, diabetes, medications that interfere with the treatment
11. Presence of significant immune compromise including HIV infection, organ transplant patients on immunosuppression, chronic lymphocytic leukaemia
12. Patients who are breastfeeding, pregnant or plan to be pregnant
13. Presence of terminal organ failure
14. Patients with moderate or severe renal impairment (GFR<60 mL/min)
15. Types of cancer that are not part of this study
16. The following types of patients are not excluded:
   a. Patients who are taking low-dose aspirin
   b. Patients who are on taking β-blockers, ACEIs or ATRBs

### Data Collection

Data to be collected includes:
1. Demographic data of the patient including gender, age, co-morbidities (e.g., ischaemic heart disease, stroke, asthma, diabetes), smoking history, alcohol abuse, medications including the name/type/dosage of RAS modulators, aspirin and other NSAIDs, and anti-diabetic treatment. Any allergy and any contraindication to medications being used for the proposed study
2. Details of the cancer before the original treatment(s) including TNM stage, clinical stage, histology grade, perinueral invasion, lymphovascular invasion
3. Details of previous treatment(s) with dates: surgery+/−radiotherapy+/−chemotherapy+/−biologic agents
4. Dates and details of response to previous treatment(s) including the presence and loco-regional recurrence and/or site(s) distant metastasis
5. Re-staging details of the cancer at relapse including PET CT findings.
6. Patient's performance status assessed by Karnofsky score
7. Documentation of the response to RAS modulation: pre- and serial measurements during treatment:
   i. Staging PET/CT:
      For GBM, pre- and 3 and 6 months, and 12 months following initiation of treatment, if indicated, i.e., the patient has improved or is stable when compared to their baseline state.
      For all other cancer types: pre- and 6 and 18 months, and 3 years following initiation of treatment if indicated, i.e., the patient has improved or is stable when compared to their baseline state.
   ii. Serial blood samples for routine blood tests:
      Renal function (electrolytes and creatinine): pre-, and 2 weeks after initiation or change of dosage of aliskirin, cilazapril or losartan
   iii. Serial blood samples: pre- and 3-monthly for 24 months following initiation of treatment; and then 4 monthly for 2 years and then 6-monthly for further 1 year, for:
      Routine blood tests: full blood count, liver function tests including GGT levels⁴¹
      Blood samples to be stored at the Gillies McIndoe Research Institute Tissue Bank (GMRITB) for future unspecified research (FUR)
8. Clinical examination (including postural BP measurement) and serial quality of life assessments: pre-, 6, and 18 months following initiation of treatment and then annually until exit of the study or completion of the study up to 3 years from initiation of treatment, using Anderson's Questionnaires for all patients, and EORTC's ALQ-C15-PAL (V1)⁴² where available for specific cancer sites; or the RAND⁴³ Questionnaires
9. Death: date and cause of death
10. Exit the trial and the reason(s)

Participants would be invited to consider giving consent for the data and tissue samples to be used for FUR. Such tissue samples will be stored at the Gillies McIndoe Research Institute Tissue Bank (GMRITB) approved by the by the Northern Health and Disability Committee (approval #12NTB42). The data from the participants, will be identifiable by their NHI number but will otherwise be anonymised, and may be used for FUR and retained within the GMRI.

### Treatment Regimen

Because there are multiple steps within the RAS pathway where control (inhibition) can be exerted, this study is designed to block as many of these steps as possible to reduce the production of angiotensin peptides (FIG. 1). Medications that inhibit these different sites in the system are to be employed in a stepwise manner. Treatment will be initiated and titrated until the optimum dose as stated in the study protocol is achieved and as tolerated by the patients.

The medications to be used in this study include:
1. Cilazapril, an ACEI, to block the action of ACE which increases the production ATII
2. Aliskirin, is a renin blocker that converts AGN to ATI. It needs to be taken at the same time each morning with food
3. Celecoxib, an inhibitor of COX-2 which promotes the conversion of the non-active pro-renin to the active renin, by upregulation of PRR. It is to be taken twice daily
4. Curcumin is a well-established antagonist of COX-2 and the protease, cathepsin. Therefore, its inclusion will reduce the conversion of pro-renin to renin. Curcumin is an active ingredient of a natural product, Turmeric. The inclusion of piperine (an active ingredient of pepper) in the formulation increases the bioavailability of Curcumin⁴⁴. The formulation chosen for this study is listed on: http://nz.iherb.com/Doctor-s-Best-Hiqh-Absoription-Curcumin-with-C3-Complex-and-BioPerine-1-000-mg-120-Tablets/12137
5. Metformin, which blocks the IGF/IGFR1 pathway that promotes the conversion of the non-active pro-renin to the active renin
6. Propranolol that inhibits the production of the pro-renin.
7. Losartan, blocks the action of ATII on ATIIR1, only to be used if patient does not tolerate ACEI.

The treatment regimen includes initiation, escalation and maintenance of the oral medications.

### Initiation and Escalation (See Table X for Dosing Regimen):

All medications will be administered orally.

If the patient is already taking an ACEi, it will be changed to an equivalent dose of cilazapril which may be 1.25 mg, 2.5 mg or 5 mg once daily. A conversion guide is included in Table 1 as follows:

TABLE 1
ACE Inhibitor Conversion Chart

| | Approximate | | | Usual | |
|---|---|---|---|---|---|
| | Dose | | | maximum | |
| Drug | Equivalence | | Once daily dosing | dose | |
| Cilazapril | 1.25 | mg | Yes | 5 | mg |
| Captopril | 6.25 | mg | No - usually BD or TDS | 150 | mg |
| Enalaprilmaleate | 2.5 | mg | Yes (can be BD) | 40 | mg |
| Lisinopril | 5 | mg | Yes | 80 | mg |
| Perindoprilerbumine | 1 | mg | Yes | 8 | mg |
| Quinapril | 5 | mg | Yes (can be BD) | 40 | mg |

BD: twice daily; TDS: three times daily
Aliskiren (150 mg once daily)~^{Δ}, celecoxib (100 mg twice daily), curcumin with piperine (500 mg twice daily, or once daily if patient develops bloating) are added. (Grapefruit and grapefruit juice are contraindicated in patients taking aliskiren).
After 2 weeks, the dosage of aliskiren is increased (to 150 mg twice daily)~^{Δ} and metformin (250 mg twice daily) is introduced
After 2 weeks, propranolol 40 mg twice daily^{#} is introduced and the dosage of metformin is increased (to 500 mg twice daily)
After 2 weeks, the dosage of propranolol is increased (to propranolol LA 160 mg once daily)^{#}
After 2 weeks, if the patient is already on cilazapril, the dosage is increased to 5 mg once daily~^{Δ} Otherwise add cilazapril (1.25 mg once daily)~^{Δ}
After 2 weeks, the dosage of cilazapril is increased to 2.5 mg once daily~^{Δ}
After another 2 weeks, cilazapril is increased to 5 mg once daily~^{Δ}
If the patient cannot tolerate cilazapril 2.5 mg, it is stopped, and losartan (50 mg once daily)~^{Δ} is introduced. The dosage of losartan is increased (to 100 mg once daily)~^{Δ} after 2 weeks
~If systolic BP is ≥100 mmHg and the patient is asymptomatic.
^{#}If systolic BP is ≥100 mmHg and heart rate is ≥50/minute and the patient is asymptomatic
^{Δ}Renal function is performed 2 weeks after initiation or changes of dosage of aliskiren, cilazapril or losartan.

### Dosing Management if Adverse Effects Occur:

Certain adverse effects (e.g., angioedema in patients on cilazapril) would necessitate cessation of the medication. A dry cough associated with cilazapril would result in the cilazapril been substituted for losartan. For patients who develop minor adverse effects e.g., cold hands and excessive fatigue on propranolol, the dosage would be decreased.

An exemplary dosing regimen is presented in the Table 2 as follows:

TABLE 2
Dosing Regimen

| | | | Curcumin with | | | |
|---|---|---|---|---|---|---|
| Week | Aliskiren | Celecoxib | Piperine | Metformin | Propranolol | Cilazapril |
| 0 | 150 mg | 100 mg | 500-1000 mg | | | |
| | once daily | twice daily | once to twice daily | | | |
| 2 | 150 mg | 100 mg | 500-1000 mg | 250 mg | | |
| | twice daily | twice daily | once to twice daily | twice daily | | |
| 4 | 150 mg | 100 mg | 500-1000 mg | 500 mg | | |
| | twice daily | twice daily | once to twice daily | twice daily | | |
| 6 | 150 mg | 100 mg | 500-1000 mg | 500 mg | 40 mg | |
| | twice daily | twice daily | once to twice daily | twice daily | twice daily | |
| 8 | 150 mg | 100 mg | 500-1000 mg | 500 mg | 160 mg | |
| | twice daily | twice daily | once to twice daily | twice daily | LA once daily | |
| 10 | 150 mg | 100 mg | 500-1000 mg | 500 mg | 160 mg | 1.25 mg |
| | twice daily | twice daily | once to twice daily | twice daily | LA once daily | once daily |
| 12 | 150 mg | 100 mg | 500-1000 mg | 500 mg | 160 mg | 2.5 mg |
| | twice daily | twice daily | once to twice daily | twice daily | LA once daily | once daily |
| 14 + | 150 mg | 100 mg | 500-1000 mg | 500 mg | 160 mg | 5 mg |
| onwards | twice daily | twice daily | once to twice daily | twice daily | LA once daily | once daily |

### Maintenance:

The treatment is maintained for the entire duration of the study, or ceased because of: side effects, it does not benefit the patient, or if the patient exits the study. Cost of the medications will be covered by the sponsor of this study beyond the duration of the study, up to 5 years from the initiation of treatment.

### Side Effects of the Medications

Propranolol

Common (1-9.9%):

General: Fatigue and/or lassitude (often transient).

Cardiovascular: Bradycardia, cold extremities, Raynaud's phenomenon. CNS: Sleep disturbances, nightmares.

Uncommon (0.1-0.9%):

Gastrointestinal: Gastrointestinal disturbance, such as nausea, vomiting, diarrhoea.

Rare (0.01-0.09%)

General: dizziness.

Blood: Thrombocytopaenia.

Cardiovascular: heart failure deterioration, precipitation of heart block, postural hypotension, which may be associated with syncope, exacerbation of intermittent claudication.

CNS: Hallucinations, psychoses, mood changes, confusion, memory loss.

Skin: Purpura, alopecia, psoriasiform skin reactions, exacerbation of psoriasis, skin rashes. Neurological: Paraesthesia. Eyes: Dry eyes, visual disturbances.

Respiratory: Bronchospasm may occur in patients with bronchial asthma or a history of asthmatic complaints, sometimes with fatal outcome.

Very rare (<0.01%):

Endocrine system: Hypoglycaemia in neonates, infants, children, elderly patients, patients on haemodialysis, patients on concomitant antidiabetic therapy, patients with prolonged fasting and patients with chronic liver disease has been reported.

Investigations: A increased antinuclear antibodies has been observed, however the clinical relevance of this is not clear.

Nervous system: Isolated reports of myasthenia gravis like syndrome or exacerbation of myasthenia gravis have been reported.

Discontinuance of the medicine should be considered if, according to clinical judgement, the well-being of the patient is adversely affected by any of the above reactions. Cessation of therapy with a beta-blocker should be gradual. In the rare event of intolerance, manifested as bradycardia and hypotension, the medicine should be withdrawn and, if necessary, treatment for overdosage instituted.

Aliskiren

Common:

Gastrointestinal: diarrhoea (2.3%)

Musculoskeletal: musculoskeletal symptom

Neurologic: dizziness, headache (2.4% to 6.2%)

Renal: Serum blood urea nitrogen raised, serum creatinine raised

Serious:

Cardiovascular: hypotension

Endocrine metabolic: hyperkalaemia (0.9%)

Immunologic: anaphylaxis, hypersensitivity reaction

Musculoskeletal: increased creatinine kinase level (1%)

Neurologic: seizure

Renal: renal impairment

Other: angioedema (0.06%)

Aspirin

Common: Increased bleeding tendencies, dyspepsia.

Uncommon: Urticaria, rhinitis, dyspnoea

Rare: Thrombocytopenia, agranulocytosis, aplastic anaemia, hypersensitivity reactions, angio-oedema, allergic oedema, anaphylactic reactions including shock.

Celecoxib

Common:

Cardiovascular: Hypertension (2-12%)

Gastrointestinal: Diarrhoea (4-10%), nausea (3-7%)

Neurological: headache (10-15%)

Serious:

Cardiovascular: Myocardial infarction (0.1% to 1.9%), Torsades de pointes, Ventricular hypertrophy (0.1% to 1%)

Dermatologic: Erythema multiforme, Erythroderma, Generalized exanthematous pustulosis, acute, Stevens-Johnson syndrome, Toxic epidermal necrolysis

Endocrine metabolic: Hyperkalaemia

Gastrointestinal: Gastrointestinal haemorrhage (less than 0.1%), Gastrointestinal perforation (less than 0.1%), Gastrointestinal ulcer, Inflammatory disorder of digestive tract

Hematologic: Haemorrhage, Thrombosis (1.2%)

Hepatic: Fulminant hepatitis, Hepatotoxicity (Rare), Increased liver enzymes (0.1% to 1.9%), Liver failure.

Immunologic: Anaphylactoid reaction, Drug reaction with eosinophilia and systemic symptoms

Neurologic: Cerebrovascular accident

Renal: Acute renal failure, Injury of kidney

Respiratory: Asthma, Bronchospasm (0.1% to 1.9%)

Cilazapril

Common: Fatigue, hypotension, dyspepsia, nausea and other gastrointestinal disturbances, headache, rash and coughing

Uncommon: Tachycardia, palpitations and chest pain

Rare: Skin rashes including erythema multiforme and toxic epidermal necrolysis may occur. Photosenstivity, alopecia and other hypersensitivity reactions.

Metformin

Common: Mild gastrointestinal symptoms (such as diarrhoea, nausea, vomiting, loss of appetite) are the most frequent reactions to metformin (>1/10), especially during the initial treatment period. These symptoms are generally transient and resolve spontaneously during continued treatment.

Very Rare: Lactic acidosis is a very rare (<1/10,000) but serious metabolic complication that can occur due to metformin accumulation during treatment A decrease of vitamin B12 absorption with a decrease in serum levels has been observed in patients treated long-term with metformin. Skin and subcutaneous tissue disorders. Mild erythema, pruritus and urticaria have been reported in some hypersensitive individuals. Nervous system disorders Common Metallic taste (3%). Hepatobiliary disorders. Isolated reports of liver function test abnormalities or hepatitis resolving upon metformin discontinuation.

Curcumin

Common: Stomach upset, nausea, dizziness, bitter taste, dermatitis or diarrhoea

Very Rare: Abnormal heart rhythm

Losartan

Common: Cold or flu symptoms such as stuffy nose, sneezing, sore throat, fever, muscle cramps, pain in the legs or back, stomach pain, diarrhoea, headache, dizziness, tired feeling, sleep problems (insomnia)

### Contraindications to the Medications

The contraindications to the medications included in the proposed study are as follows:

Celecoxib

Celecoxib is contraindicated in patients with hypersensitivity to aspirin or any other NSAID-which includes those in whom attacks of asthma, angioedema, urticaria or rhinitis have been precipitated by aspirin or any other NSAID; ischaemic heart disease, cerebrovascular disease, peripheral arterial disease, and mild to severe heart failure; active gastro-intestinal ulceration or bleeding; inflammatory bowel disease; coronary artery bypass graft surgery; manufacturer advises avoid in sulfonamide hypersensitivity, severe renal impairment (GFR<30 mL/min).

Caution is given in the use of celecoxib in the elderly; coagulation defects; connective-tissue disorders; patients at risk of peptic ulceration or gastro-intestinal bleeding, history of cardiac failure, left ventricular dysfunction, hypertension in patients with oedema for any other reason, and in patients with risk factors for cardiovascular events; renal impairment; long term use of some NSAIDs may reduce female fertility (reversible on stopping).

Aliskiren

Aliskiren is contraindicated in patients with previous hypersensitivity to aliskiren, diabetic patients taking ARBs or ACEIs because of the risk of renal impairment, hypotension and hyperkalemia. Aliskiren is to be avoided with the use of ARBs or ACEIs in patients with renal impairment (GFR<60 mL/min).

Curcumin

Curcumin is contraindicated in patients with hypersensitivity to turmeric, gall bladder obstruction, gall stones, hyperacidity or gastrointestinal ulcers, obstruction of bile passages, pregnancy, and lactation.

Cilazapril

Cilazapril is contraindicated in patients who are hypersensitive to the active substance and any other ACEIs. Like other ACEis, cilazapril is contraindicated in patients with a history of angioedema related to previous treatment with an ACEi. Cilazapril, like other ACEIs, is contraindicated in pregnancy and lactation.

Metformin

Metformin is contraindicated in patients with ketoacidosis, significant renal impairment (avoid if eGFR<15 mL/min/1.73 m²), undergoing general anaesthesia for surgery (suspend on morning of surgery, support with insulin if required, restart when renal function returns to baseline). Caution is given in the use of metformin in patients with renal impairment.

Propranolol

Propranolol is contraindicated in patients with hypersensitivity to the active substance or to any of the excipients. Propranolol as with other β-blockers must not be used in patients with any of the following conditions: known hypersensitivity to the substance; bradycardia, cardiogenic shock; hypotension, metabolic acidosis, after prolonged fasting, severe peripheral arterial circulatory disturbances, second or third degree heart block, sick sinus syndrome, untreated pheochromocytoma, uncontrolled heart failure, and Prinzmetal's angina. Propranolol must not be used in patients prone to hypoglycaemia, i.e., patients after prolonged fasting or patients with restricted counter-regulatory reserves. Patients with restricted counter regulatory reserves may have reduced autonomic and hormonal responses to hypoglycaemia which includes glycogenolysis, gluconeogenesis and/or impaired modulation of insulin secretion. Patients at risk for an inadequate response to hypoglycaemia includes individuals with malnutrition, prolonged fasting, starvation, chronic liver disease, diabetes and concomitant use of drugs which block the full response to catecholamines.

Losartan

Losartan is contraindicated in patients with hypersensitivity to Losartan or other ARBs, in pregnancy and severe hepatic impairment. It should not be administered with aliskerin in patients with diabetes.

### Significant Drug Interactions

Significant drug interactions for each medication being used for this study are listed under each medication, below:

Celecoxib

Cidofovir, mifamurtide, adefovir, amiloride, ciclosporin, quinolones (e.g. ciprofloxacin, norfloxacin), dasatinib, apixaban, clopidogrel, dabigatran, warfarin, enoxaparin, methotrexate, lithium, prasugrel, acetazolamide, tricyclic antidepressants (e.g. amitriptyline, nortriptyline), desmopressin, nicorandil, tacrolimus, spironolactone, serotonin noradrenaline re-uptake inhibitors (e.g. venlafaxine), selective serotonin re-uptake inhibitors (e.g. citalopram, fluoxetine, sertraline, escitalopram), NSAID's (e.g., diclofenac, ibuprofen), alendronate, probenecid, thiazide diuretics (e.g bendroflumethazide, indapamide, chlorthalidone), loop diuretics (e.g. furosemide, bumetanide), digoxin, corticosteroids (e.g. prednisone, dexamethasone, methylprednisolone), clozapine.

Aliskiren

ACEIs, ARBs, itraconazole, cyclosporin, furosemide, rifampicin, potassium supplements, spironolactone, amiloride. Grapefruit and grapefruit juice.

Curcumin

Enoxaparin, warfarin, dabigatran, clopidogrel, prasugrel, apixaban, alteplase, and tenecteplase.

Cilazapril

NSAID's, furosemide, thiazide diuretics, spironolactone, amiloride, allopurinol, azathioprine, baclofen, cyclosporin, enoxaparin, heparin, potassium supplements, lithium, sirolimus, tacrolimus, tolvaptan, trimethoprim, co-trimoxazole, doxazosin.

Metformin

Acetazolamide, amisulpride, aripiprazole, beclomethasone (inhaled), bendrofluazide, bortezomib, budesonide (inhalation/systemic), capecitabine, chlorpromazine, thiazide diuretics, clonidine, citalopram, escitalopram, clozapine, dexamethasone, prednisone, prednisolone, haloperidol, isoniazid, olanzapine, paroxetine, fluoxetine, topiramate

Propranolol

Theophylline, clonidine, rizatriptan, verapamil, amiodarone, baclofen, chlorpromazine, diltiazem, flecainide, levothyroxine, pseudoephedrine, terbinafine (systemic), thalidomide, thioridazine, tranylcypromine, xylometazoline (systemic), antacids (mylanta or quickeze).

Losartan

Potassium sparing diuretics (e.g., spironolactone, triamterene, amiloride), potassium supplements, or salt substitutes containing potassium may lead to increases in serum potassium. Serum lithium levels should be monitored carefully if Lithium salts are to be co-administered with ARBs. NSAIDs including selective COX-2 inhibitors, especially in patients with impaired renal function.

### Other Medications/Treatments to be Continued During the Study

During the study, apart from the medication(s) that are to be converted as listed under “Treatment Regimen” patients will continue their normal medications administered for other conditions.

### Other Medicines Not Permitted During Study

Refer to “Exclusion Criteria.”

### Safety and Monitoring

The medications are of low risk and the adverse effects and safety profiles are well established. However, safety is of the utmost priority and measures will be undertaken to prevent or minimise risks to the patients. The patient's safety will be actively managed by the researchers being mindful of the side effects of medications used for the study as described above, as well as contraindications to the medications, and significant drug interactions listed above.

Once enrolled, communication with the participants' general practitioners and other relevant health professionals involved in the patient's care by the investigators will be established. The participants will be provided an information sheet including the ability to report side effects of the medications which are described herein. As part of the study the patients will be monitored regularly for side effects and response to the treatment. This will involve regular clinical assessments, measurements of blood pressure (including postural BP), pulse rate, serial blood samples (for renal function, etc), and PET CT scans, as outlined on p4 of the Study Protocol.

At each visit the participants will be routinely questioned for any difficulty in taking the medication and questioned to assess compliance of the treatment. Due to the number of additional medications that the patient would be required to take and to aid compliance to the regime the trial medications will be blister packed. These will be dated and the participants will be asked to bring these in to the follow up sessions. This will allow for a visual check on compliance, i.e. tablet count.

### Study Plan and Timing of Procedures

The study and timing of procedures covering the recruitment, enrolment, treatment and monitoring for the duration of the study are provided in the document ‘Study and Timing of Procedures’ as follows:

| | Phone | | | | | | | | |
|---|---|---|---|---|---|---|---|---|---|
| | Call | Treatment | | | | | | | |
| Visit number | 0 | 1 | 2 | 3 | 4 | 5 | 6 | 7 | 8 |
| Weeks (W)/months (M)/years | 3-4 weeks | 1-2 weeks | 0 | 2 W | 4 W | 8 W | 10 W | 3 M | 6 M |
| (Y) from start of treatment | before | before | | | | | | | |
| | treatment | treatment | | | | | | | |
| Screening to exclude non- | X | | | | | | | | |
| eligible patients | | | | | | | | | |
| Patient's performance status | X | | | | | | | | X |
| Quality of Life questionnaires | X | | | | | | | | X |
| Assessment for eligibility to | X | | | | | | | | |
| enter study - screening | | | | | | | | | |
| Demography | X | | | | | | | | |
| Medical/surgical history incl. | X | X | | | | | | | |
| previous treatments | | | | | | | | | |
| Examination including blood | | X | X | X | X | X | X | X | X |
| pressure and pulse rate | | | | | | | | | |
| Confirm eligibility to enter study | | X | | | | | | | |
| Written informed consent | | X | | | | | | | |
| PET CT scan | | X | | | | | | | X |
| Blood sample taken | | X | X | X | X | X | X | X | X |
| Record of any adverse effects | | | | X | X | X | X | X | X |
| Compliance of medications | | | | X | X | X | X | X | X |

| Visit number | 9 | 10 | 11 | 12 | 13 | 14 | 15 | 16 |
|---|---|---|---|---|---|---|---|---|
| Months (M)/years (Y) from | 9 M | 1 Y | 15 M | 18 M | 2 Y | 28 M | 32 M | 3 Y |
| start of treatment | | | | | | | | |
| Patient's performance status | | | | X | | | | X |
| Quality of Life questionnaires | | | | X | | | | X |
| Examination including blood | X | X | X | X | X | X | X | X |
| pressure and pulse rate | | | | | | | | |
| PET CT scan | | | | X | | | | X |
| Blood sample taken | X | X | X | X | X | X | X | X |
| Record of any adverse effects | X | X | X | X | X | X | X | X |
| Compliance of medications | X | X | X | X | X | X | X | X |

### Handling of Adverse Events and Emergencies During Study

Any adverse events (AEs) and serious adverse event (SAEs) will be collected and reviewed by a Data Safety Management Board (DSMB) at 6 monthly intervals.

The participants will be contacted by the research nurse by phone about the results of the blood tests and PET CT scan within one week of the results becoming available.

The participants will also be informed if the investigators became aware of new important information, such as SAEs, and as any changes will be provided in a revised info sheet for the participants who will be asked to consent for on-going study.

The participants will be able to choose if they wish to receive a copy of the overall study results, after the whole study has been completed. An HDEC approved study summary letter will be provided to these participants and participants will be invited to contact the researchers if they have any questions or concerns.

### Criteria for Exclusion During Trial

The participants will exit the study because of his/her decision, death, leaving the country, or significant deterioration of general health during the study period as assessed with a Karnofsky score of <60.

The sponsor of the study will review the data from the study participants at 6 monthly intervals. More than one SAE in a patient will trigger a review of patient safety and consideration whether to terminate the study. The lack of efficacy during the interim analysis of the results and/or evidence of treatment safety concerns will lead to termination of the study.

### Recruitment

As the potential participants are part of a vulnerable population with terminal cancer, measures will be put in place to mitigate this potential risk. For example, the patients will be approached by a third party (e.g., research nurse) rather than the investigators. The catchment of the participants will primarily be Central and Mid-Central regions with a combined population of over 1 million but will include the entire country.

### Duration of Study

The duration of the study will be years, as this duration will determine the effectiveness of the proposed treatment regime. However, patients who have been successfully treated at the end of this trial period will wish to continue with the medications beyond the end of the study. Although not part of the trial their progress will be followed by the investigators.

### Patients Who Drop Out of the Study

Patients who drop out of the study will continue to be under the care of their GP, and their GP will be notified so that they will be responsible for their ongoing treatment. By having 25 subjects in each cancer group, the study is sufficiently powered statistically that a drop out level of more than 40% will still allow meaningful outcomes to be determined. Drop outs will be replaced to ensure at least 15 patients from each cancer group complete the study.

### Statistical Analyses

This study will use a before and after comparison (pre-test versus post-test) of the data using the patient as their own controls. The most powerful statistical test for this purpose is the t-test for related samples. The sample size calculation for this method requires the following parameters:
α=The threshold probability for rejecting the null hypothesis (Type I Error) 0.050 for a two-sided test. But as the patients are not expected to get better a one sided value is used 0.100.
β=The probability of failing to reject the null hypothesis (Type II Error).
E=The Effect size. A common convention is to use the standardised value 0.500 where this is unknown.
S is the Standard Deviation of the outcome in the population obtained from survival studies. Here it is 2.000 (estimated from published survival studies).
S(Δ)=The Standard Deviation of the CHANGE in the outcome.
   Where this is unknown it is found using the formula S(Δ)=S(2(1−r_{within})^{1/2}
   Here 2.000 is substituted for S, and r_{within} is 0.875. The result is A (=1.000).

The standard normal deviate for α is Z_{α}=1.645, and for β=Z_{β}=0.842. When A=1.000 and B=(Zα+Zβ)²=6.183 & C=(E/S(Δ))²=0.250.

Then AB/C=24.73 (25) cases for the present study. (For a two sided test the sample would have required 31 cases)^{45,46}.

Any quantifiable measure will be tested using the t-test for related samples. This includes results from the Quality of Life questionnaires, performance status of the patients, tumour number and size and activity (measured by PET CT) of the primary site and/or metastases.

### EXAMPLE 5: EXEMPLARY DRUG COMBINATIONS

### Drug Combination #1

Applicants prepared a drug combination for administration to a patient, the drug combination comprising Aspirin, Propanolol and Curcumin, each packaged separately and optionally inclusive of a pharmaceutically acceptable excipient. In one alternative, the drug combination included Piperidine, either packaged separately or formulated together with Curcumin.

Drug Combination #1A: Drug Combination #1 wherein Aspirin is present in an amount of up to 300 mg; Propanolol is present in an amount of up to 320 mg; Curcumin is present in an amount of up to 8000 mg.

Drug Combination #1B: Drug Combination #1 or Drug Combination #1A, formulated for oral administration to a patient.

### Drug Combination #2

Applicants prepared a drug combination for administration to a patient, the drug combination comprising Aspirin, Curcumin and Aliskiren each packaged separately and optionally inclusive of a pharmaceutically acceptable excipient. In one alternative, the drug combination included Piperidine, either packaged separately or formulated together with Curcumin.

Drug Combination #2A: Drug Combination #2, wherein Aspirin is present in an amount of up to 300 mg; Curcumin is present in an amount of up to 8000 mg; Aliskiren is present in an amount of up to 300 mg.

Drug Combination #2B: Drug Combination #2 or Drug Combination #2A, formulated for oral administration to a patient.

### Drug Combination #3

Applicants prepared a drug combination for administration to a patient, the drug combination comprising Celecoxib, Propanolol and Curcumin, each packaged separately and optionally inclusive of a pharmaceutically acceptable excipient. In one alternative, the drug combination included Piperidine, either packaged separately or formulated together with Curcumin.

Drug Combination #3A: Drug Combination #3, wherein Celecoxib is present in an amount of up to 200 mg; Propanolol is present in an amount of up to 320 mg; Curcumin is present in an amount of up to 8000 mg.

Drug Combination #3B: Drug Combination #3 or Drug Combination #3A, formulated for oral administration to a patient.

### Drug Combination #4

Applicants prepared a drug combination for administration to a patient, the drug combination comprising Curcumin, Propanolol, Aspirin and Quinapril each packaged separately and optionally inclusive of a pharmaceutically acceptable excipient. In one alternative, the drug combination included Piperidine, either packaged separately or formulated together with Curcumin.

Drug Combination #4A: Drug Combination #4, wherein Curcumin is present in an amount of up to 8000 mg; Propanolol is present in an amount of up to 320 mg; Aspirin is present in an amount of up to 300 mg; Quinapril is present in an amount of up to 40 mg.

Drug Combination #4B: Drug Combination #4 or Drug Combination #4A, formulated for oral administration to a patient.

### Drug Combination #5

Applicants prepared a drug combination for administration to a patient, the drug combination comprising Aliskiren, Celecoxib and Curcumin, each packaged separately and optionally inclusive of a pharmaceutically acceptable excipient. In one alternative, the drug combination included Piperidine, either packaged separately or formulated together with Curcumin.

Drug Combination #5A: Drug Combination #5, wherein Aliskiren is present in an amount of up to 150 mg; Celecoxib is present in amount of up to 200 mg; Curcumin is present in amount of up to 1000 mg.

Drug Combination #5B: Drug Combination #5A, wherein Curcumin is present in two discrete doses of 500 mg.

Drug Combination #5C: Drug Combination #5B, formulated for oral administration to a patient.

### Drug Combination #6

Applicants prepared a drug combination for administration to a patient, the drug combination comprising Aliskiren, Celecoxib, Curcumin and Metformin, each packaged separately and optionally inclusive of a pharmaceutically acceptable excipient. In one alternative, the drug combination included Piperidine, either packaged separately or formulated together with Curcumin.

Drug Combination #6A: Drug Combination #6, wherein Aliskiren is present in an amount of up to 300 mg; Celecoxib is present in amount of up to 200 mg; Curcumin is present in amount of up to 1000 mg; Metformin is present in an amount of up to 500-1000 mg.

Drug Combination #6B: Drug Combination #6A, wherein Aliskiren is present in two discrete doses of up to 150 mg.

Drug Combination #6C: Drug Combination #6B, wherein Curcumin is present in two discrete doses of up to 500 mg.

Drug Combination #6D: Drug Combination #6C, wherein where Metformin is present in an amount of up to 500 mg, it is present in two discrete doses of up to 250 mg.

Drug Combination #6E: Drug Combination #6C, wherein where Metformin is present in an amount of up to 1000 mg, it is present in two discrete doses of up to 500 mg.

Drug Combination #6F: Drug Combination #6D, formulated for oral administration to a patient.

Drug Combination #6G: Drug Combination #6E, formulated for oral administration to a patient.

### Drug Combination #7

Applicants prepared a drug combination for administration to a patient, the drug combination comprising Aliskiren, Celecoxib, Curcumin, Metformin and Propanolol, each packaged separately and optionally inclusive of a pharmaceutically acceptable excipient. In one alternative, the drug combination included Piperidine, either packaged separately or formulated together with Curcumin.

Drug Combination #7A: Drug Combination #7, wherein Aliskiren is present in an amount of up to 300 mg; Celecoxib is present in amount of up to 200 mg; Curcumin is present in amount of up to 1000 mg; Metformin is present in an amount of up to 1000 mg; Propanolol is present in an amount of up to 80-160 mg.

Drug Combination #7B: Drug Combination #7A, wherein Aliskiren is present in two discrete doses of up to 150 mg.

Drug Combination #7C: Drug Combination #7B, wherein Curcumin is present in two discrete doses of 500 mg.

Drug Combination #7D: Drug Combination #7C, wherein Metformin is present in two discrete doses of up to 500 mg.

Drug Combination #7E: Drug Combination #7D, wherein where Propanolol is present in an amount of up to 80 mg, it is present in two discrete doses of up to 40 mg.

Drug Combination #7F: Drug Combination #7D, wherein where Propanolol is present in an amount of up to 160 mg, it is present in a single dose.

Drug Combination #7G Drug Combination #7E, formulated for oral administration to a patient.

Drug Combination #7H: Drug Combination #7F, formulated for oral administration to a patient.

### Drug Combination #8

Applicants prepared a drug combination for administration to a patient, the drug combination comprising Aliskiren, Celecoxib, Curcumin, Metformin, Propanolol and Cilazapril, each packaged separately and optionally inclusive of a pharmaceutically acceptable excipient. In one alternative, the drug combination included Piperidine, either packaged separately or formulated together with Curcumin.

Drug Combination #8A: Drug Combination #8, wherein Aliskiren is present in an amount of up to 300 mg; Celecoxib is present in amount of up to 200 mg; Curcumin is present in amount of up to 1000 mg; Metformin is present in an amount of up to 1000 mg; Propanolol is present in an amount of up to 160 mg; Cilazapril is present in amount of up to 1.25-5.0 mg.

Drug Combination #8B: Drug Combination #8A, wherein Aliskiren is present in two discrete doses of up to 150 mg.

Drug Combination #8C: Drug Combination #8B, wherein Curcumin is present in two discrete doses of up to 500 mg.

Drug Combination #8D: Drug Combination #8C, wherein Metformin is present in two discrete doses of up to 500 mg.

Drug Combination #8E: Drug Combination #8D, wherein Cilazapril is present in an amount of 1.25 mg.

Drug Combination #8F: Drug Combination #8D, wherein Cilazapril is present in an amount of 2.5 mg.

Drug Combination #8G: Drug Combination #8D, wherein Cilazapril is present in an amount of 5 mg.

Drug Combination #8H: Drug Combination #8E, formulated for oral administration to a patient.

Drug Combination #8I: Drug Combination #8F, formulated for oral administration to a patient.

Drug Combination #8J: Drug Combination #8G, formulated for oral administration to a patient.

### Drug Combination #9

Applicants prepared a drug combination for administration to a patient, the drug combination comprising Aliskiren, Celecoxib, Curcumin, Metformin, Propanolol and Losartan, each packaged separately and optionally inclusive of a pharmaceutically acceptable excipient. In one alternative, the drug combination included Piperidine, either packaged separately or formulated together with Curcumin.

Drug Combination #9A: Drug Combination #9, wherein Aliskiren is present in an amount of up to 150 mg; Celecoxib is present in amount of up to 200 mg; Curcumin is present in amount of up to 1000 mg; Metformin is present in an amount of up to 500-1000 mg; Propanolol is present in an amount of up to 80-160 mg; Losartan is present in amount of up to 100 mg.

Drug Combination #9B: Drug Combination #9A, wherein Aliskiren is present in two discrete doses of up to 150 mg.

Drug Combination #9C: Drug Combination #9B, wherein Curcumin is present in two discrete doses of up to 500 mg.

Drug Combination #9D: Drug Combination #9C, wherein Metformin is present in two discrete doses of up to 500 mg.

Drug Combination #9E: Drug Combination #9D, wherein Losartan is present in an amount of 100 mg.

Drug Combination #9F: Drug Combination #9E, formulated for oral administration to a patient.

### EXAMPLE 6: EXEMPLARY PHARMACEUTICAL COMPOSITIONS

### Pharmaceutical Composition #1

Applicants prepared a pharmaceutical composition for administration to a patient, the pharmaceutical composition comprising Aspirin, Propanolol and Curcumin, together with a pharmaceutically acceptable excipient. In one alternative, the pharmaceutical composition included Piperidine.

Pharmaceutical Composition #1A: Pharmaceutical Composition #1 wherein Aspirin is present in an amount of up to 300 mg; Propanolol is present in an amount of up to 320 mg; Curcumin is present in an amount of up to 8000 mg.

Pharmaceutical Composition #1B: Pharmaceutical Composition #1A, formulated for oral administration to a patient.

### Pharmaceutical Composition #2

Applicants prepared a pharmaceutical composition for administration to a patient, the pharmaceutical composition comprising Aspirin, Curcumin and Aliskiren, together with a pharmaceutically acceptable excipient. In one alternative, the pharmaceutical composition included Piperidine.

Pharmaceutical Composition #2A: Pharmaceutical Composition #2, wherein Aspirin is present in an amount of up to 300 mg; Curcumin is present in an amount of up to 8000 mg; Aliskiren is present in an amount of up to 300 mg.

Pharmaceutical Composition #2B: Pharmaceutical Composition #2A, formulated for oral administration to a patient.

### Pharmaceutical Composition #3

Applicants prepared a pharmaceutical composition for administration to a patient, the pharmaceutical composition comprising Celecoxib, Propanolol and Curcumin, together with a pharmaceutically acceptable excipient. In one alternative, the pharmaceutical composition included Piperidine.

Pharmaceutical Composition #3A: Pharmaceutical Composition #3, wherein Celecoxib is present in an amount of up to 200 mg; Propanolol is present in an amount of up to 320 mg; Curcumin is present in an amount of up to 8000 mg.

Pharmaceutical Composition #3B: Pharmaceutical Composition #3A, formulated for oral administration to a patient.

### Pharmaceutical Composition #4

Applicants prepared a pharmaceutical composition for administration to a patient, the pharmaceutical composition comprising Curcumin, Propanolol, Aspirin and Quinapril, together with a pharmaceutically acceptable excipient. In one alternative, the pharmaceutical composition included Piperidine.

Pharmaceutical Composition #4A: Pharmaceutical Composition #4, wherein Curcumin is present in an amount of up to 8000 mg; Propanolol is present in an amount of up to 320 mg; Aspirin is present in an amount of up to 300 mg; Quinapril is present in an amount of up to 40 mg.

Pharmaceutical Composition #4B: Pharmaceutical Composition #4A, formulated for oral administration to a patient.

### Pharmaceutical Composition #5

Applicants prepared a pharmaceutical composition for administration to a patient, the pharmaceutical composition comprising Aliskiren, Celecoxib and Curcumin, together with a pharmaceutically acceptable excipient. In one alternative, the pharmaceutical composition included Piperidine.

Pharmaceutical Composition #5A: Pharmaceutical Composition #5, wherein Aliskiren is present in an amount of up to 150 mg; Celecoxib is present in amount of up to 200 mg; Curcumin is present in amount of up to 500 mg.

Pharmaceutical Composition #5B: Pharmaceutical Composition #5A, formulated for oral administration to a patient.

### Pharmaceutical Composition #6

Applicants prepared a pharmaceutical composition for administration to a patient, the pharmaceutical composition comprising Aliskiren, Celecoxib, Curcumin and Metformin, together with a pharmaceutically acceptable excipient. In one alternative, the pharmaceutical composition included Piperidine.

Pharmaceutical Composition #6A: Pharmaceutical Composition #6, wherein Aliskiren is present in an amount of up to 150 mg; Celecoxib is present in amount of up to 200 mg; Curcumin is present in amount of up to 500 mg; Metformin is present in an amount of up to 250 mg.

Pharmaceutical Composition #6B: Pharmaceutical Composition #6, wherein Aliskiren is present in an amount of up to 150 mg; Celecoxib is present in amount of up to 200 mg; Curcumin is present in amount of up to 500 mg; Metformin is present in an amount of up to 500 mg.

Pharmaceutical Composition #6C: Pharmaceutical Composition #6A, formulated for oral administration to a patient.

Pharmaceutical Composition #6D: Pharmaceutical Composition #6B, formulated for oral administration to a patient.

### Pharmaceutical Composition #7

Applicants prepared a pharmaceutical composition for administration to a patient, the pharmaceutical composition comprising Aliskiren, Celecoxib, Curcumin, Metformin and Propanolol, together with a pharmaceutically acceptable excipient. In one alternative, the pharmaceutical composition included Piperidine.

Pharmaceutical Composition #7A: Pharmaceutical Composition #7, wherein Aliskiren is present in an amount of up to 150 mg; Celecoxib is present in amount of up to 200 mg; Curcumin is present in amount of up to 500 mg; Metformin is present in an amount of up to 500 mg; Propanolol is present in an amount of up to 40 mg.

Pharmaceutical Composition #7B: Pharmaceutical Composition #7B, wherein Aliskiren is present in an amount of up to 150 mg; Celecoxib is present in amount of up to 200 mg; Curcumin is present in amount of up to 500 mg; Metformin is present in an amount of up to 500 mg; Propanolol is present in an amount of up to 160 mg.

Pharmaceutical Composition #7C: Pharmaceutical Composition #7A, formulated for oral administration to a patient.

Pharmaceutical Composition #7D: Pharmaceutical Composition #7B, formulated for oral administration to a patient.

### Pharmaceutical Composition #8

Applicants prepared a pharmaceutical composition for administration to a patient, the pharmaceutical composition comprising Aliskiren, Celecoxib, Curcumin, Metformin, Propanolol and Cilzapril, together with a pharmaceutically acceptable excipient. In one alternative, the pharmaceutical composition included Piperidine.

Pharmaceutical Composition #8A: Pharmaceutical Composition #8, wherein Aliskiren is present in an amount of up to 150 mg; Celecoxib is present in amount of up to 200 mg; Curcumin is present in amount of up to 500 mg; Metformin is present in an amount of up to 500 mg; Propanolol is present in an amount of up to 160 mg; Cilazapril is present in amount of up to 1.25 mg.

Pharmaceutical Composition #8B: Pharmaceutical Composition #8, wherein Aliskiren is present in an amount of up to 150 mg; Celecoxib is present in amount of up to 200 mg; Curcumin is present in amount of up to 500 mg; Metformin is present in an amount of up to 500 mg; Propanolol is present in an amount of up to 160 mg; Cilazapril is present in amount of up to 2.5 mg.

Pharmaceutical Composition #8C: Pharmaceutical Composition #8, wherein Aliskiren is present in an amount of up to 150 mg; Celecoxib is present in amount of up to 200 mg; Curcumin is present in amount of up to 500 mg; Metformin is present in an amount of up to 500 mg; Propanolol is present in an amount of up to 160 mg; Cilazapril is present in amount of up to 5 mg.

Pharmaceutical Composition #8D: Pharmaceutical Composition #8A, formulated for oral administration to a patient.

Pharmaceutical Composition #8E: Pharmaceutical Composition #8B, formulated for oral administration to a patient.

Pharmaceutical Composition #8F: Pharmaceutical Composition #8C, formulated for oral administration to a patient.

### Pharmaceutical Composition #9

Applicants prepared a pharmaceutical composition for administration to a patient, the pharmaceutical composition comprising Aliskiren, Celecoxib, Curcumin, Metformin, Propanolol and Losartan, together with a pharmaceutically acceptable excipient. In one alternative, the pharmaceutical composition included Piperidine.

Pharmaceutical Composition #9A: Pharmaceutical Composition #9, wherein Aliskiren is present in an amount of up to 150 mg; Celecoxib is present in amount of up to 200 mg; Curcumin is present in amount of up to 500 mg; Metformin is present in an amount of up to 500 mg; Propanolol is present in an amount of up to 160 mg; Losartan is present in amount of up to 100 mg.

Pharmaceutical Composition #9B: Pharmaceutical Composition #9A, formulated for oral administration to a patient.

### Pharmaceutical Composition #10

Applicants prepared a pharmaceutical composition for administration to a patient, the pharmaceutical composition comprising Aliskiren together with a pharmaceutically acceptable excipient.

Pharmaceutical Composition #10A: Pharmaceutical Composition #10, wherein Aliskiren is present in an amount of up to 150 mg.

Pharmaceutical Composition #10B: Pharmaceutical Composition #10A, formulated for oral administration to a patient.

### Pharmaceutical Composition #11

Applicants prepared a pharmaceutical composition for administration to a patient, the pharmaceutical composition comprising Curcumin together with a pharmaceutically acceptable excipient. In one alternative, the pharmaceutical composition included Piperidine.

Pharmaceutical Composition #11A: Pharmaceutical Composition #11, wherein Curcumin is present in an amount of up to 500 mg.

Pharmaceutical Composition #118: Pharmaceutical Composition #11A, formulated for oral administration to a patient.

### Pharmaceutical Composition #12

Applicants prepared a pharmaceutical composition for administration to a patient, the pharmaceutical composition comprising Curcumin together with a pharmaceutically acceptable excipient.

Pharmaceutical Composition #12A: Pharmaceutical Composition #12, wherein Metformin is present in an amount of up to 250 mg.

Pharmaceutical Composition #12B: Pharmaceutical Composition #12A, formulated for oral administration to a patient.

### Pharmaceutical Composition #13

Applicants prepared a pharmaceutical composition for administration to a patient, the pharmaceutical composition comprising Metformin together with a pharmaceutically acceptable excipient.

Pharmaceutical Composition #13A: Pharmaceutical Composition #13, wherein Metformin is present in an amount of up to 500 mg.

Pharmaceutical Composition #13B: Pharmaceutical Composition #13A, formulated for oral administration to a patient.

### Pharmaceutical Composition #14

Applicants prepared a pharmaceutical composition for administration to a patient, the pharmaceutical composition comprising Propanolol together with a pharmaceutically acceptable excipient.

Pharmaceutical Composition #14A: Pharmaceutical Composition #14, wherein Propanolol is present in an amount of up to 40 mg.

Pharmaceutical Composition #14B: Pharmaceutical Composition #14A, formulated for oral administration to a patient.

### Pharmaceutical Composition #15

Applicants prepared a pharmaceutical composition for administration to a patient, the pharmaceutical composition comprising Aliskiren, Curcumin and Metformin together with a pharmaceutically acceptable excipient.

Pharmaceutical Composition #15A: Pharmaceutical Composition #15, wherein Aliskiren is present in an amount of up to 150 mg, Curcumin is present in an amount of up to 500 mg and Metformin is present in an amount of up to 250 mg.

Pharmaceutical Composition #15B: Pharmaceutical Composition #15, wherein Aliskiren is present in an amount of up to 150 mg, Curcumin is present in an amount of up to 500 mg and Metformin is present in an amount of up to 500 mg.

Pharmaceutical Composition #15C: Pharmaceutical Composition #15A, formulated for oral administration to a patient.

Pharmaceutical Composition #15D: Pharmaceutical Composition #15B, formulated for oral administration to a patient.

### Pharmaceutical Composition #16

Applicants prepared a pharmaceutical composition for administration to a patient, the pharmaceutical composition comprising Aliskiren, Curcumin, Metformin and Propanolol together with a pharmaceutically acceptable excipient.

Pharmaceutical Composition #16A: Pharmaceutical Composition #16, wherein Aliskiren is present in an amount of up to 150 mg, Curcumin is present in an amount of up to 500 mg, Metformin is present in an amount of up to 500 mg and Propanolol is present in an amount of up to 40 mg.

Pharmaceutical Composition #16B: Pharmaceutical Composition #16A, formulated for oral administration to a patient.

### EXAMPLE 7: EXEMPLARY ARTICLES OF MANUFACTURE/THERAPEUTIC KITS

### Article of Manufacture #1

Applicants prepared an article of manufacture comprising Drug Combination #5C together with instructions for how to administer the various drugs to a patient simultaneously, separately or sequentially via the oral route, including instructions for twice daily oral dosing of Curcumin to the patient.

### Article of Manufacture #2

Applicants prepared an article of manufacture comprising Drug Combination #6F together with instructions for how to administer the various drugs to a patient simultaneously, separately or sequentially via the oral route, including instructions for twice daily oral dosing of Aliskiren, Curcumin and Metformin to the patient.

### Article of Manufacture #3

Applicants prepared an article of manufacture comprising Drug Combination #6G together with instructions for how to administer the various drugs to a patient simultaneously, separately or sequentially via the oral route, including instructions for twice daily oral dosing of Aliskiren, Curcumin and Metformin to the patient.

### Article of Manufacture #4

Applicants prepared an article of manufacture comprising Drug Combination #7G together with instructions for how to administer the various drugs to a patient simultaneously, separately or sequentially via the oral route, including instructions for twice daily oral dosing of Aliskiren, Curcumin, Metformin and Propanolol to the patient.

### Article of Manufacture #5

Applicants prepared an article of manufacture comprising Drug Combination #7H together with instructions for how to administer the various drugs to a patient simultaneously, separately or sequentially via the oral route, including instructions for twice daily oral dosing of Aliskiren, Curcumin and Metformin to the patient.

### Article of Manufacture #6

Applicants prepared an article of manufacture comprising Drug Combination #8H together with instructions for how to administer the various drugs to a patient simultaneously, separately or sequentially via the oral route, including instructions for twice daily oral dosing of Aliskiren, Curcumin and Metformin to the patient.

### Article of Manufacture #7

Applicants prepared an article of manufacture comprising Drug Combination #8I together with instructions for how to administer the various drugs to a patient simultaneously, separately or sequentially via the oral route, including instructions for twice daily oral dosing of Aliskiren, Curcumin and Metformin to the patient.

### Article of Manufacture #8

Applicants prepared an article of manufacture comprising Drug Combination #8J together with instructions for how to administer the various drugs to a patient simultaneously, separately or sequentially via the oral route, including instructions for twice daily oral dosing of Aliskiren, Curcumin and Metformin to the patient.

### Article of Manufacture #9

Applicants prepared an article of manufacture comprising Pharmaceutical Composition #5B and Pharmaceutical Composition #11B, together with instructions for how to administer the pharmaceutical compositions to a patient via the oral route, including instructions for twice daily oral dosing of Curcumin.

### Article of Manufacture #10

Applicants prepared an article of manufacture comprising Pharmaceutical Composition #6C, Pharmaceutical Composition #10B, Pharmaceutical Composition 11B, and Pharmaceutical Composition 12B, together with instructions for how to administer the pharmaceutical compositions to a patient via the oral route, including instructions for twice daily oral dosing of Aliskiren, Curcumin and Metformin.

### Article of Manufacture #11

Applicants prepared an article of manufacture comprising Pharmaceutical Composition #6D, Pharmaceutical Composition #10B, Pharmaceutical Composition 11B, and Pharmaceutical Composition 13B, together with instructions for how to administer the pharmaceutical compositions to a patient via the oral route, including instructions for twice daily oral dosing of Aliskiren, Curcumin and Metformin.

### Article of Manufacture #12

Applicants prepared an article of manufacture comprising Pharmaceutical Composition #7C, Pharmaceutical Composition #10B, Pharmaceutical Composition 11B, Pharmaceutical Composition 13B and Pharmaceutical Composition 14B, together with instructions for how to administer the pharmaceutical compositions to a patient via the oral route, including instructions for twice daily oral dosing of Aliskiren, Curcumin, Metformin and Propanolol.

### Article of Manufacture #13

Applicants prepared an article of manufacture comprising Pharmaceutical Composition #7D, Pharmaceutical Composition #10B, Pharmaceutical Composition 111B, Pharmaceutical Composition 13B, together with instructions for how to administer the pharmaceutical compositions to a patient via the oral route, including instructions for twice daily oral dosing of Aliskiren, Curcumin and Metformin.

### Article of Manufacture #14

Applicants prepared an article of manufacture comprising Pharmaceutical Composition #8D, Pharmaceutical Composition #10B, Pharmaceutical Composition 11B, Pharmaceutical Composition 13B, together with instructions for how to administer the pharmaceutical compositions to a patient via the oral route, including instructions for twice daily oral dosing of Aliskiren, Curcumin and Metformin.

### Article of Manufacture #15

Applicants prepared an article of manufacture comprising Pharmaceutical Composition #8E, Pharmaceutical Composition #10B, Pharmaceutical Composition 11B, Pharmaceutical Composition 13B, together with instructions for how to administer the pharmaceutical compositions to a patient via the oral route, including instructions for twice daily oral dosing of Aliskiren, Curcumin and Metformin.

### Article of Manufacture #16

Applicants prepared an article of manufacture comprising Pharmaceutical Composition #8F, Pharmaceutical Composition #10B, Pharmaceutical Composition 11B, Pharmaceutical Composition 13B, together with instructions for how to administer the pharmaceutical compositions to a patient via the oral route, including instructions for twice daily oral dosing of Aliskiren, Curcumin and Metformin.

### Article of Manufacture #17

Applicants prepared an article of manufacture comprising Pharmaceutical Composition #6C and Pharmaceutical Composition #15C, together with instructions for how to administer the pharmaceutical compositions to a patient via the oral route, including instructions for twice daily oral dosing of Aliskiren, Curcumin and Metformin.

### Article of Manufacture #18

Applicants prepared an article of manufacture comprising Pharmaceutical Composition #6D and Pharmaceutical Composition #15D, together with instructions for how to administer the pharmaceutical compositions to a patient via the oral route, including instructions for twice daily oral dosing of Aliskiren, Curcumin and Metformin.

### Article of Manufacture #19

Applicants prepared an article of manufacture comprising Pharmaceutical Composition #7C and Pharmaceutical Composition #16B, together with instructions for how to administer the pharmaceutical compositions to a patient via the oral route, including instructions for twice daily oral dosing of Aliskiren, Curcumin, Metformin and Propanolol.

### Article of Manufacture #20

Applicants prepared an article of manufacture comprising Pharmaceutical Composition #7D and Pharmaceutical Composition #15D, together with instructions for how to administer the pharmaceutical compositions to a patient via the oral route, including instructions for twice daily oral dosing of Aliskiren, Curcumin and Metformin.

### Article of Manufacture #21

Applicants prepared an article of manufacture comprising Pharmaceutical Composition #8D and Pharmaceutical Composition #15D, together with instructions for how to administer the pharmaceutical compositions to a patient via the oral route, including instructions for twice daily oral dosing of Aliskiren, Curcumin and Metformin.

### Article of Manufacture #22

Applicants prepared an article of manufacture comprising Pharmaceutical Composition #8E and Pharmaceutical Composition #15D, together with instructions for how to administer the pharmaceutical compositions to a patient via the oral route, including instructions for twice daily oral dosing of Aliskiren, Curcumin and Metformin.

### Article of Manufacture #23

Applicants prepared an article of manufacture comprising Pharmaceutical Composition #8F and Pharmaceutical Composition #15D, together with instructions for how to administer the pharmaceutical compositions to a patient via the oral route, including instructions for twice daily oral dosing of Aliskiren, Curcumin and Metformin.

### EXAMPLE 8: EXEMPLARY TREATMENT REGIME

Cancer patients underwent treatment using the various Drug Combinations, Pharmaceutical Compositions and Articles of Manufacture as described herein. In particular, patients having either oral cavity squamous cell carcinoma (OCSCC), locally advanced and/or metastatic head and neck skin squamous cell carcinoma (HNsSCC), glioblastoma multiforme (GBM) and malignant melanoma (MM) were recruited. Refer to Example 5.

In a non-limiting example according to the present invention, recruited patients underwent the following treatment regime:
1. Article of Manufacture #1 or Article of Manufacture #9 was administered to the patient daily for a period of about two weeks; then
2. Article of Manufacture #2 or Article of Manufacture #10 or Article of Manufacture #17 was administered to the patient daily for about a further two weeks; then
3. Article of Manufacture #3 or Article of Manufacture #11 or Article of Manufacture #18 was administered to the patient daily for about a further two weeks; then
4. Article of Manufacture #4 or Article of Manufacture #12 or Article of Manufacture #19 was administered to the patient daily for about a further two weeks; then
5. Article of Manufacture #5 or Article of Manufacture #13 or Article of Manufacture #20 was administered to the patient daily for about a further two weeks; then
6. Article of Manufacture #6 or Article of Manufacture #14 or Article of Manufacture #21 was administered to the patient daily for about a further two weeks; then
7. Article of Manufacture #7 or Article of Manufacture #15 or Article of Manufacture #22 was administered to the patient daily for about a further two weeks; then
8. Article of Manufacture #8 or Article of Manufacture #16 or Article of Manufacture #23 was administered to the patient daily for about a further two weeks, or as further required.

A person skilled in the art will recognise that the time frames associated with the dosing regimen exemplified here and elsewhere is approximate, and will vary from patient to patient depending on response to the treatment. Further, a clinician may opt to exchange certain drugs in the Drug Combination or change the Pharmaceutical Composition depending on the side effects observed for any given patient.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### REFERENCES

1. Chiou S H., et al. Positive correlations of Oct-4 and Nanog in oral cancer stem-like cells and high-grade oral squamous cell carcinoma. Clin Cancer Res. 2008; 14: 4085-95.
2. Shakhova O., et al. Testing the cancer stem cell hypothesis in melanoma: the clinics will tell. Cancer Lett. 2013; 338: 74-81.
3. Bradshaw A., et al. Cancer stem cell hierachy in glioblastoma multiforme. Front Surg.

DOI: 10.3389/fsurg.2016.00021.
4. Pan C X., et al. Implications of cancer stem cells in the treatment of cancer. Future Oncol. 2006; 2: 723-31.
5. http://google.com/patents/WO2016024870A1?cl=en
6. Baillie R., et al. Cancer stem cells in moderately differentiated oral tongue squamous cell carcinoma. J Clin Pathol. doi: 10.1136/jclinpath-2015-203599.
7. Yu H H., et al. Characterization of cancer stem cells in moderately differentiated buccal mucosal squamous cell carcinoma. Front Surg. DOI: 10.3389/fsurg.2016.00046.
8. Bradshaw A., et al. Cancer stem cells in glioblastoma multiforme. Front Surg (submitted).
9. Neves F A., et al. Cathepsin is a prorenin processing enzyme. Hypertension 1996; 27: 514-7.
10. Cousin C., Potential role of the (pro)renin receptor in cardiovascular and kidney diseases. J Nephrol. 2010; 23: 508-13.
11. Wang F., et al. COX-2 mediates angiotensin II-induced (pro)renin receptor expression in the rat renal medulla. Renal Physiology. 2014; 307: F25-32.
12. Holmer S R., et al. Marked suppression of renin levels by beta-receptor blocker in patients treated with standard heart failure therapy: a potential mechanism of benefit from beta-blockade. J Intern Med. 2001; 249: 167-72.
13. Standen P., et al. Maternal insulin-like growth factor 1 and 2 differentially affect the renin-angiotensin system during pregnancy in the guinea pig. Growth Horm IGF Res. 2015; 25: 141-7.
14. Malaquarnera R., et al. The emerging role of insulin and insulin-like growth factor signalling in cancer stem cells. Front Endocrinol. 2014; 5: 10.
15. Sarfstein R., et al. Metformin downregulates the insulin/IGF-1 signalling pathway and inhibits different uterine serous carcinoma (USC) cells proliferation and migration in p53-dependent or -independent manners. PLoS. 2013; 8: e61537.
16. Ager E I., et al. The renin-angiotensin system and malignancy. Carcinogenesis. 2008; 29: 1675-84.
17. Shibayama., et al. (Pro)renin receptor is crucial for Wnt/α-catenin-dependent genesis of pancreatic ductal adenocarcinoma. Sci Rep. 2015; 5: 8854.
18. Itinteang T., et al. Cancer stem cells in moderately differentiated oral tongue squamous cell carcinoma express components of the renin-angiotensin system. J Clin Pathol. DOI: 10.1136/jclinpath-2016-203736.
19. Featherston T., et al. Cancer stem cells in moderately differentiated buccal squamous cell carcinoma express components of the renin-angiotensin system. Front Surg. (submitted).
20. Bradshaw A., et al. Glioblastoma multifrome cancer stem cells express components of the renin-angiotensin system. Front Surg. (submitted).
21. Itinteang T., et al. Expression of cathepsins B, D, and G in infantile hemangioma.

Front Surg. DOI: 10.3389/fsurg.2015.00026.
22. Christian J B., et al. Association of ACE inhibitors and angiotensin receptor blockers with keratinocyte cancer prevention in the randomized VATTC trial. JNCI J Natl Cancer Inst. 2008; 100: 1223-32.
23. Chang P Y., Et al. Propranolol reduces cancer risk: a population-based cohort study. Medicine. 2015; 94: e1097.
24. Awtry E H., Et al. Asprin. Circ. 2000; 101: 1206-18.
25. Ferrandez A., et al. COX-2 and colorectal cancer. Curr Pharm Des. 2003; 9: 2229-51.
26. Januel E., et al. Impact of renin-angiotensin system blockade on clinical outcome in glioblastoma. Eur J Neurol. 2015; 22: 1304-9.
27. Powe D G., et al. Targeted therapies: using β-blockers to inhibit breast cancer progression. Nat Rev Clin Oncol. 2011; 8: 511-512.
28. Watkins J L., et al. Clinical impact of selective and nonselective beta-blockers on survival in patients with ovarian cancer. Cancer. 2015; 121: 3444-51.
29. Kozangoglu I., et al. New indication for therapeutic potential of an old well-known drug (propranolol) for multiple myeloma. J Cancer Res Clin Oncol. 2013; 139: 327-35.
30. Yang W., et al. Cathepsin expression and the correlation with clinical aspects of oral squamous cell carcinoma. PLoS ONE. 2016; 11: e0152165.
31. Ravish I., Curcumin as inhibitor of mammalian cathepsin, cathepsin H, acid phosphatase and alkaline phosphatase: a correlation with pharmacological activities. Medicinal Chemistry Research. 2014; 23: 2847-55.
32. Wilken R., et al. Curcumin: a review of anti-cancer properties and therapeutic activty in head and neck squamous cell carcinoma. Molecular Cancer. DOI: 10.1186/1476-4598-10-12.
33. Kucab J E., et al. Role of IGF-1R in mediating breast cancer invasion and metastasis. Breast Dis. 2003; 17: 41-7.
34. Talarico G., et al. Asprin and atenolol enhance metformin activity against breast cancer by targeting both neoplastic and microenvirnment cells. Scientfic Reports. 2016; 6: 18673.
35. White H., et al. Quasi-experimental design and methods. UNICEF Methodological Briefs, Impact Evaluation; 2014:8.
36. Hemelrijck., et al. Gamma-glutamyltransferase and risk of cancer in a cohort of 545,460 persons—the Swedish AMORIS study. Eur J Cancer. 2011; 47: 2033-41.
37. Denaro N., et al. State-of-the-art and emerging treatment options in the management of head and neck cancer: news from 2013. Oncology 2014; 86: 212-229.
38. Tas F. Metastatic behaviour in melanoma: timing, pattern, survival and influencing factors. J Oncol 2012: 647684.
39. Stupp R., et al. radiotherapy plus concomitant and adjuvant temozolomide for glioblastoma. N Engl J Med. 2005; 352: 987-96.
40. Karnofsky D A., et al. The use of the nitrogen mustards in the palliative treatment of carcinoma. With particular reference to bronchogenic carcinoma. Cancer 1948; 1: 634-656.
41. Hemelrijck., et al. Gamma-glutamyltransferase and risk of cancer in a cohort of 545,460 persons—the Swedish AMORIS study. Eur J Cancer. 2011; 47: 2033-41.
42. Aaronson N K., et al. European organisation for research and treatment of cancer QLQ-C30A quality of life instrument for use in international clinical trials in oncology. J Natl cancer Int. 1993; 85: 365-76.
43. Ware J E., et al. The MOS 36-item short form health survey (RAND) (SF-36). Med Care 1992; 30: 473-83.
44. Shoba G., et al. Influence of piperine on the pharmacokinetics of curcumin in animals and human volunteers. Planta Med. 1998; 64: 353-356
45. Rosner B. Fundamentals of biostatistics. 4th ed. Duxbury Press 1995; p221.
46. Hulley S., et al. Designing clinical research. 4th ed. Lippincourt, Williams & Wilkins. 2013.

1.-8. (canceled)

9. A method for treating and/or managing cancer or a non-cancerous tumour in a patient in need thereof, the method comprising administering to the patient a therapeutically effective amount of a drug combination comprising (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide, (1E,6E)-1,7-Bis(4-hydroxy-3-methoxyphenyl)hepta-1,6-diene-3,5-dione, N,N-dimethylimidodicarbonimidic diamide and (RS)-1-(1-methylethylamino)-3-(1-naphthyloxy)propan-2-ol, or pharmaceutically acceptable salts of any one or more of the foregoing.

10.-15. (canceled)

16. The method of claim 9, wherein the drug combination further comprises [3S-[2[R*(R)],3R*]]-2-[2-[[1-Ethoxycarbonyl)-3-phenylpropyl]amino]-1-oxopropyl]-1,2,3,4-tetrahydro-3-isoquinolinecarboxylic acid monohydrochloride or a pharmaceutically acceptable salt thereof.

17. The method of claim 9, wherein the drug combination further comprises (4S,7S)-7-[[(2S)-1-Ethoxy-1-oxo-4-phenylbutan-2-yl]amino]-6-oxo-1,2,3,4,7,8,9,10-octahydropyridazino[1,2-a]diazepine-4-carboxylic acid or 2-butyl-4-chloro-1-{[2′-(1H-tetrazol-5-yl)biphenyl-4-yl]methyl}-1H-imidazol-5-yl)methanol or pharmaceutically acceptable salts thereof.

18. The method of claim 16, wherein the drug combination further comprises 4-[5-(4-Methylphenyl)-3-(trifluoromethyl)pyrazol-1-yl]benzenesulfonamide or a pharmaceutically acceptable salt thereof.

19. The method of claim 9, wherein the drug combination further comprises 1-[5-(1,3-Benzodioxol-5-yl)-1-oxo-2,4-pentadienyl]piperidine.

20. The method of claim 16, wherein the drug combination further comprises 1-[5-(1,3-Benzodioxol-5-yl)-1-oxo-2,4-pentadienyl]piperidine.

21. The method of claim 17, wherein the drug combination further comprises 1-[5-(1,3-Benzodioxol-5-yl)-1-oxo-2,4-pentadienyl]piperidine.

22. The method of claim 18, wherein the drug combination further comprises 1-[5-(1,3-Benzodioxol-5-yl)-1-oxo-2,4-pentadienyl]piperidine.

23. The method of claim 9, wherein the compounds are formulated for simultaneous, separate or sequential administration to a patient in need thereof.

24. The method of claim 9, wherein the compounds are formulated for oral administration to a patient in need thereof.

25. The method of claim 18, comprising:
(i) orally administering (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide or a pharmaceutically acceptable salt thereof to the patient a maximum daily amount of up to 150 mg;
(ii) orally administering 4-[5-(4-methylphenyl)-3-(trifluoromethyl)pyrazol-1-yl]benzenesulfonamide or a pharmaceutically acceptable salt thereof to the patient a maximum daily amount of up to 200 mg;
(iii) orally administering (1E,6E)-1,7-Bis(4-hydroxy-3-methoxyphenyl)hepta-1,6-diene-3,5-dione or a pharmaceutically acceptable salt thereof to the patient a maximum daily amount of up to about 2000 mg;
(iv) orally administering N,N-dimethylimidodicarbonimidic diamide or a pharmaceutically acceptable salt thereof to the patient a total daily amount of up to about 1000 mg;
(v) orally administering (RS)-1-(1-methylethylamino)-3-(1-naphthyloxy)propan-2-ol or a pharmaceutically acceptable salt thereof to the patient a maximum daily amount of up to about 120 mg; and
(vi) orally administering [3S-[2[R*(R)],3R*]]-2-[2-[[1-Ethoxycarbonyl)-3-phenylpropyl]amino]-1-oxopropyl]-1,2,3,4-tetrahydro-3-isoquinolinecarboxylic acid monohydrochloride or a pharmaceutically acceptable salt thereof to the patient a maximum daily amount of up to about 20 mg.

26. The method of claim 18, comprising:
(i) for a first period of about a week:
(a) orally administering (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide or a pharmaceutically acceptable salt thereof to the patient a maximum daily amount of up to about 150 mg;
(b) orally administering (1E,6E)-1,7-Bis(4-hydroxy-3-methoxyphenyl)hepta-1,6-diene-3,5-dione or a pharmaceutically acceptable salt thereof to the patient a maximum daily amount of up to about 2000 mg;
(c) orally administering N,N-dimethylimidodicarbonimidic diamide or a pharmaceutically acceptable salt thereof to the patient a maximum daily amount of up to about 250 mg; and
(d) orally administering (RS)-1-(1-methylethylamino)-3-(1-naphthyloxy)propan-2-ol or a pharmaceutically acceptable salt thereof to the patient a maximum daily amount of up to about 80 mg;
(ii) for a second period of about a week:
(a) orally administering (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide or a pharmaceutically acceptable salt thereof to the patient a maximum daily amount of up to about 150 mg;
(b) orally administering (1E,6E)-1,7-Bis(4-hydroxy-3-methoxyphenyl)hepta-1,6-diene-3,5-dione or a pharmaceutically acceptable salt thereof to the patient a maximum daily amount of up to about 2000 mg;
(c) orally administering N,N-dimethylimidodicarbonimidic diamide or a pharmaceutically acceptable salt thereof to the patient a maximum daily amount of up to about 500 mg; and
(d) orally administering (RS)-1-(1-methylethylamino)-3-(1-naphthyloxy)propan-2-ol or a pharmaceutically acceptable salt thereof to the patient a maximum daily amount of up to about 120 mg; and
(iii) for a third period of about a week:
(a) orally administering (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide or a pharmaceutically acceptable salt thereof to the patient a maximum daily amount of up to about 150 mg;
(b) orally administering (1E,6E)-1,7-Bis(4-hydroxy-3-methoxyphenyl)hepta-1,6-diene-3,5-dione or a pharmaceutically acceptable salt thereof to the patient a maximum daily amount of up to about 2000 mg;
(c) orally administering N,N-dimethylimidodicarbonimidic diamide to the patient a maximum daily amount of about 750 mg; and
(d) orally administering (RS)-1-(1-methylethylamino)-3-(1-naphthyloxy)propan-2-ol or a pharmaceutically acceptable salt thereof to the patient a maximum daily amount of about 120 mg; and
(e) orally administering [3S-[2[R*(R)],3R*]]-2-[2-[[1-Ethoxycarbonyl)-3-phenylpropyl]amino]-1-oxopropyl]-1,2,3,4-tetrahydro-3-isoquinolinecarboxylic acid monohydrochloride or a pharmaceutically acceptable salt thereof to the patient a maximum daily amount of up to about 5 mg; and
(iv) for a fourth period of about a week:
(a) orally administering (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide or a pharmaceutically acceptable salt thereof to the patient a maximum daily amount of about 150 mg;
(b) orally administering (1E,6E)-1,7-Bis(4-hydroxy-3-methoxyphenyl)hepta-1,6-diene-3,5-dione or a pharmaceutically acceptable salt thereof to the patient a maximum daily amount of about 2000 mg;
(c) orally administering N,N-dimethylimidodicarbonimidic diamide or a pharmaceutically acceptable salt thereof to the patient about 1000 mg;
(d) orally administering (RS)-1-(1-methylethylamino)-3-(1-naphthyloxy)propan-2-ol or a pharmaceutically acceptable salt thereof to the patient a maximum daily amount of about 120 mg; and
(e) orally administering [3S-[2[R*(R)],3R*]]-2-[2-[[1-Ethoxycarbonyl)-3-phenylpropyl]amino]-1-oxopropyl]-1,2,3,4-tetrahydro-3-isoquinolinecarboxylic acid monohydrochloride or a pharmaceutically acceptable salt thereof to the patient a maximum daily amount of up to about 10 mg; and
(v) for a fifth period of about a week or longer, as therapeutically required by the patient:
(a) orally administering (2S,4S,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide or a pharmaceutically acceptable salt thereof to the patient a maximum daily amount of about 150 mg;
(b) orally administering (1E,6E)-1,7-Bis(4-hydroxy-3-methoxyphenyl)hepta-1,6-diene-3,5-dione or a pharmaceutically acceptable salt thereof to the patient a maximum daily amount of about 2000 mg;
(c) orally administering N,N-dimethylimidodicarbonimidic diamide or a pharmaceutically acceptable salt thereof to the patient a maximum daily amount of about 1000 mg;
(d) orally administering (RS)-1-(1-methylethylamino)-3-(1-naphthyloxy)propan-2-ol or a pharmaceutically acceptable salt thereof to the patient a maximum daily amount of about 120 mg; and
(e) orally administering [3S-[2[R*(R)],3R*]]-2-[2-[[1-Ethoxycarbonyl)-3-phenylpropyl]amino]-1-oxopropyl]-1,2,3,4-tetrahydro-3-isoquinolinecarboxylic acid monohydrochloride or a pharmaceutically acceptable salt thereof to the patient a maximum daily amount of up to about 20 mg; and
(f) orally administering 4-[5-(4-Methylphenyl)-3-(trifluoromethyl)pyrazol-1-yl]benzenesulfonamide or a pharmaceutically acceptable salt thereof to the patient a maximum daily amount of up to about 200 mg.

27. The method according to claim 16, wherein the cancer is selected from the group consisting of oral cavity squamous cell carcinoma, head and neck skin squamous cell carcinoma, glioblastoma multiforme and malignant melanoma.

28. The method according to claim 17, wherein the cancer is selected from the group consisting of oral cavity squamous cell carcinoma, head and neck skin squamous cell carcinoma, glioblastoma multiforme and malignant melanoma.

29. The method according to claim 18, wherein the cancer is selected from the group consisting of oral cavity squamous cell carcinoma, head and neck skin squamous cell carcinoma, glioblastoma multiforme and malignant melanoma.

30. The method according to claim 19, wherein the cancer is selected from the group consisting of oral cavity squamous cell carcinoma, head and neck skin squamous cell carcinoma, glioblastoma multiforme and malignant melanoma.

31. The method according to claim 20, wherein the cancer is selected from the group consisting of oral cavity squamous cell carcinoma, head and neck skin squamous cell carcinoma, glioblastoma multiforme and malignant melanoma.

32. The method according to claim 21, wherein the cancer is selected from the group consisting of oral cavity squamous cell carcinoma, head and neck skin squamous cell carcinoma, glioblastoma multiforme and malignant melanoma.

33. The method according to claim 22, wherein the cancer is selected from the group consisting of oral cavity squamous cell carcinoma, head and neck skin squamous cell carcinoma, glioblastoma multiforme and malignant melanoma.

34. The method according to claim 23, wherein the cancer is selected from the group consisting of oral cavity squamous cell carcinoma, head and neck skin squamous cell carcinoma, glioblastoma multiforme and malignant melanoma.

35. The method according to claim 24, wherein the cancer is selected from the group consisting of oral cavity squamous cell carcinoma, head and neck skin squamous cell carcinoma, glioblastoma multiforme and malignant melanoma.

36. The method according to claim 25, wherein the cancer is selected from the group consisting of oral cavity squamous cell carcinoma, head and neck skin squamous cell carcinoma, glioblastoma multiforme and malignant melanoma.

37. The method according to claim 26, wherein the cancer is selected from the group consisting of oral cavity squamous cell carcinoma, head and neck skin squamous cell carcinoma, glioblastoma multiforme and malignant melanoma.

38. The method according to claim 9, wherein the cancer is selected from the group consisting of oral cavity squamous cell carcinoma, head and neck skin squamous cell carcinoma, glioblastoma multiforme and malignant melanoma.

39. The method of claim 9, wherein the drug combination further comprises 4-[5-(4-Methylphenyl)-3-(trifluoromethyl)pyrazol-1-yl]benzenesulfonamide or a pharmaceutically acceptable salt thereof.

40. The method according to claim 39, wherein the cancer is selected from the group consisting of oral cavity squamous cell carcinoma, head and neck skin squamous cell carcinoma, glioblastoma multiforme and malignant melanoma.
